(19) United States
(12) Patent Application Publication
(10) Pub. No.: US 2014/0199368 A1
(43) Pub. Date: Jul. 17, 2014

(54) INTRAESOPHAGEAL ADMINISTRATION OF TARGETED NITROXIDE AGENTS FOR PROTECTION AGAINST IONIZING IRRADIATION-INDUCED ESOPHAGITIS

(71) Applicants: Michael W. Epperly, Pittsburgh, PA (US); Xiang Gao, Pittsburgh, PA (US); Joel S. Greenberger, Sewickley, PA (US); Song Li, Wexford, PA (US); Peter Wipf, Pittsburgh, PA (US)

(72) Inventors: Michael W. Epperly, Pittsburgh, PA (US); Xiang Gao, Pittsburgh, PA (US); Joel S. Greenberger, Sewickley, PA (US); Song Li, Wexford, PA (US); Peter Wipf, Pittsburgh, PA (US)

(73) Assignee: University of Pittsburg-Of the Commonwealth System of Higher Education-Office of Technolology Manage, Pittsburgh, PA (US)

(21) Appl. No.: 13/885,391

(22) PCT Filed: Nov. 15, 2011
(86) PCT No.: PCT/US11/60750

§ 371 Date: Mar. 26, 2014

Related U.S. Application Data
(60) Provisional application No. 61/413,850, filed on Nov. 15, 2010.

(51) Int. Cl. C07D 211/94; C07K 5/087; C07D 207/46; C07F 9/22; C07D 471/08

(52) U.S. Cl. CPC C07D 211/94; C07F 9/22; C07D 471/08; C07D 207/46; C07K 5/0812
USPC 424/450; 514/315; 514/89; 514/294; 514/416; 514/21.9

(57) ABSTRACT

Provided herein are compositions and related methods useful for prevention or mitigation of ionizing radiation-induced esophagitis. The compositions comprise compounds comprising a nitroxide-containing group attached to a mitochondria-targeting group. The compounds can be cross-linked into dimers without loss of activity. The method comprises delivering a compound, as described herein, to a patient in an amount and dosage regimen effective to prevent or mitigate esophageal damage caused by radiation.

This application claims the benefit under 35 U.S.C. §119(e) to U.S. Provisional Patent Application No. 61/413,850, filed on Nov. 15, 2010, which is incorporated herein by reference in its entirety.

### STATEMENT REGARDING FEDERAL FUNDING

This invention was made with government support under Grant Nos. NIAID U19 AI68021, P50GM067082 and R01CA83876, awarded by the National Institutes of Health. The government has certain rights in the invention.

Radiation therapy of non-small cell lung cancer and esophageal cancer is accompanied by the significant side effect of esophagitis. Radiotherapy induced esophagitis also contributes to the morbidity of chemoradiotherapy of metastatic malignancies, and also limits dose escalation protocols due to dehydration, esophageal ulceration and the requirement for treatment breaks. Local therapeutic strategies to minimize esophagitis have been attempted and include swallowed administration of manganese superoxide dismutase-plasmid liposomes (MnSOD-PL). Intraesophageal administration of MnSOD-PL decreases radiation-induced esophageal cellular DNA double strand breaks (Niu Y, et al. Rad Res 173: 453-461, 2010), stem cell, esophageal ulceration, and dehydration with reduced morbidity of single fraction and fractionated thoracic irradiation in an animal model. In a recent phase I clinical trial, MnSOD-PL administration twice weekly to patients receiving seven and a half weeks chemoradiotherapy for unresectable non-small cell lung cancer was shown to be safe. A phase II clinical trial is currently in progress.

Intraesophageal administration of MnSOD-PL provides radioprotection associated with migration to the esophagus of bone marrow-derived progenitors of esophageal squamous epithelium. Due to the required 24-hour interval between the time of administration of MnSOD-PL and expression of transgene product, which allows for transgene transport to the nucleus, transcription of transgene message, protein production and localization at the mitochondria, a need for an alternative, more rapid acting radioprotector exists. MnSOD transgene product acts by dismutation of superoxide to hydrogen peroxide, thereby decreasing the availability of superoxide to combine with nitric oxide to produce the lethal radical peroxynitrite.

Nitroxide radicals, such as 4-amino-Tempo (4-AT), can be effective radioprotectors; however, high systemic doses are required to reduce toxicity. The mitochondrial localization and increased drug effectiveness of a novel Gramicidin S (GS)-derived nitroxide, JP4-039, which targets 4-AT to the mitochondria was demonstrated by linking it covalently to a peptide isostere analog of the cyclopeptide antibiotic GS.

### SUMMARY

Provided herein are novel compositions comprising a compound comprising nitroxide group-containing cargo (or “nitroxide containing group”) and a mitochondria-targeting group (or “targeting group”). Further provided herein are novel formulations of the aforementioned nitroxide-containing compositions. Also provided herein are methods of protecting the esophagus from radiation-induced damage, such as ionizing radiation-induced esophagitis, and mitigating the damage therefrom. The method comprises administering to the esophagus of a patient prior to, during or after exposure of the subject to radiation, a composition comprising an amount of a targeted nitroxide compound effective to prevent, mitigate or treat radiation injury in the subject. This method is demonstrated to successfully protect irradiated subjects from radiation-induced esophagitis.

The targeted nitroxide compound is chosen from one of:

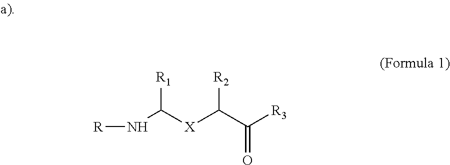

wherein X is one of

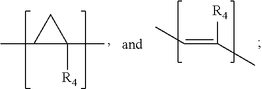

R₁ and R₂ are hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₃ is —NH—R₅, —O—R₅ or —CH₂—R₅, and R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl-, chloro- or fluoro-substituted;
b). a compound having the structure (i) R1-R2-R3 or (ii) R1, in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; and

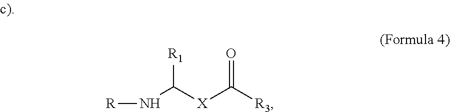

wherein X is one of

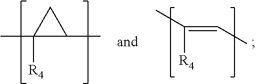

R₁ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl-, chloro- or fluoro-substituted. In one non-limiting embodiment, the compound is JP4-036. Additional targeted nitroxide compounds are described herein and in U.S. Pat. Nos. 7,718,603, 7,528,174, United States Patent Application Publication No. 20100035869, and International (PCT) Patent Application Publication Nos. WO 2010/009389 and WO 2010/009405, including XJB-5-131, XJB-5-125, XJB-5-197, XJB-7-53, XJB-7-55, XJB-7-75, JP4-049, XJB-5-208, JED-E71-37, and JED-E71-58. Uses of one or more of the described compounds for preventing or mitigating ionizing irradiation-induced esophagitis in a patient also are provided.

According to the methods provided herein, the above-described compounds are delivered to the subject by the intra-esophageal route in a liquid composition prior to, during or following exposure of the subject to ionizing radiation. A “liquid” includes, without limitation: solutions (that is, with solute dissolved in a solvent), including aqueous and non-aqueous solutions, syrups, elixirs, suspensions, colloids, homogenates, emulsions, multi-phase or multi-lamellar mixtures (for example, and without limitation, w/o (w=water, o=oil), o/w, w/o/w, and o/w/o mixtures), liposome compositions, micelle- or reverse micelle-containing compositions and flowable gels or hydrogels (that is a liquid with increased viscosity due to the presence of viscosity enhancers, such as natural or synthetic (co)polymers).

In one embodiment, the formulation is a liposome or multiphase composition prepared from a phosphatidyl choline, a non-ionic surfactant, a composition capable of forming a high axial ratio microstructure (“a HARM”) and an aqueous solvent. According to one non-limiting embodiment, the multiphase or liposome composition consists essentially of soy phosphatidyl choline, Tween 80, L-glutamic acid-1,5-dioleyl amide (approximately 4:1:1 w/w), and an aqueous solvent with 8 mg/nil JP4-039. Non-limiting examples of an aqueous solvent include water, normal (0.9%) saline and phosphate-buffered saline. The non-ionic detergent may be a polysorbate, such as Tween 80. In certain embodiments, the HARM is L-glutamic acid-1,5-dioleyl amide and/or the phosphatidyl choline may be soy phosphatidyl choline.

### BRIEF DESCRIPTION OF THE DRAWINGS

FIG. 1 provides non-limiting examples of certain nitroxides. The log P values were estimated using the online calculator of molecular properties and drug likeness on the Molinspirations Web site (www.molinspiration.com/cgi-bin/properties). TIPNO=tert-butyl isopropyl phenyl nitroxide.

FIG. 2 provides examples of structures of certain mitochondria-targeting antioxidant compounds referenced herein, and the structure of TEMPOL.

FIG. 3A is a schematic of a synthesis protocol for JP4-039. FIG. 3B provides a synthesis route for a compound of Formula 4, below.

FIGS. 4A and 4B are graphs showing GS-nitroxide compound JP4-039 increases survival of mice exposed to 9.75 Gy total body irradiation.

FIG. 5 is a graph showing that GS-nitroxide compound JP4-039 increases survival of mice exposed to 9.5 Gy total body irradiation.

FIG. 6 is a graph showing that GS-nitroxide JP4-039 is an effective hematopoietic cell radiation mitigator when delivered 24 hr after irradiation.

FIG. 7 is a graph showing that JP4-039 is an effective mitigator of irradiation damage to KM101 human marrow stromal cells.

FIGS. 8A and 8B provides structures for compounds JED-E71-37 and JED-E71-58, respectively.

FIG. 9 is a schematic showing alternative designs of nitroxide analogues.

FIG. 10 is a schematic of a synthesis protocol for various alternative designs of nitroxide analogues.

FIG. 11 is a schematic of a synthesis protocol for an alternative nitroxide moiety of 1,1,3,3-tetramethylisoindolin-2-yloxyl (TMIO).

FIG. 12 is a schematic of a synthesis protocol for an alternative nitroxide moiety of 1-methyl 2-azaadamantane N-oxyl (1-Me-AZADO).

FIG. 13. Pharmacokinetics of clearance of JP4-039 intravenously injected into C57BL/6JHNsd mice in (A) plasma and (B) lung. Mice were injected with 4 mg/kg JP4-039 in cremphor A/ethanol 50% to 50%. Serum samples were collected and assayed. Each symbol represents an individual mouse. The methods for assay of nitroxide by EPR have been published previously (Borisenko G G, et al. J Am Chem Soc 4(30): 9221-9232, 2004 and Jiang J, et al. A mitochondria-targeted triphenylphosphonium-conjugated nitroxide functions as a radioprotector/mitigator. Radiat Res 172(6): 706-717, 2009).

FIG. 14. Superior penetration of cationic multilamellar liposomes F15 containing 0.5 mole percent of Lissamine Rhodamine B-DOPE into the murine esophagus by swallowed F15 compared to control formulation that does not contain dioleoylamindo-L-glutamate. Images of esophageal cross-sections taken at 10 minutes after swallow of 4 mg/kg of protein in 100 μl formulation are shown (magnification: ×100). A, F15 formulation; B, control formulation.

FIG. 15. Quantitation of mitochondrial targeted nitroxide JP4-039 for several time points over a 60-minute period after swallow in the esophagus by EPR. The results represent mean and standard error of n=5 per group. Controls included non phycoerythrin-treated esophagi. The experimental procedures are described in Materials and Methods and in (Borisenko G G, et al. J Am Chem Soc 4(30): 9221-9232, 2004 and Jiang J, et al. A mitochondria-targeted triphenylphosphonium-conjugated nitroxide functions as a radioprotector/mitigator. Radiat Res 172(6): 706-717, 2009).

FIG. 16. Effect of JP4-039/F15 on esophageal irradiation toxicity. Female mice (15 per group) received MnSOD-PL, JP4-039 in F15 formulation, F15 formulation, or 29 Gy upper body irradiation alone as described in Materials and Methods. P-Values showed a significant effect of pre-irradiation intraesophageal MnSOD-PL or JP4-039/F15 compared to F15 emulsion alone against 29 Gy.

FIG. 17. Effect of GFP+ male marrow intravenous injection and JP4-039/F15 on esophageal irradiation toxicity. Female mice (15 per group) received 29 Gy upper body irradiation on day 0, then on day 5 they received 1×10⁷ GFP+ marrow cells intravenously from male donors. p-Values showed a significant effect of pre-irradiation intraesophageal MnSOD-PL or JP4-039/F15 on increasing the survival; p=0.0315 and p=0.0462, respectively.

FIG. 18. Detection of GFP+ marrow-derived cells in the irradiated mouse esophagus after intravenous transplant. Mice were irradiated to 29 Gy to the esophagus on day 0, and then injected with 1×10⁷ GFP+ marrow cells intravenously on day 5 according to published methods (Epperly M W, et al. Int J Cancer (Radiat Oncol Invest) 96: 221-233, 2001; Epperly M W, et al. In Vivo 19: 997-1004, 2005; and Epperly M W, et al. Protection of esophageal stem cells from ionizing irradiation by MnSOD-plasmid liposome gene therapy. In Vivo 19: 965-974, 2005). Five esophagus samples were removed from each animal in the various subgroups on days 1 (A), 3 (B), 7 (C), 14 (D), 28 (E) and 60 (F) after marrow injection. Samples of excised esophagi were prepared as single cell suspensions and then analyzed by cell sorting for GFP+ cells/10⁶ esophagus cells. Each symbol represents one esophagus.

FIG. 19. Effect of JP4-039 in F15 on percent survival in mice receiving (A) 29 Gy thoracic irradiation or (B) four daily fractions of 11.5 Gy thoracic irradiation.

FIG. 20. Effect of JP4-039 on survival following 20 Gy thoracic irradiation in mice with 3LL tumors.

FIG. 21. Effect of JP4-039 on survival in mice exposed to (A) 9.5 Gy and (B) 9.15 Gy total-body irradiation.

FIG. 22. (A) Fluorochrome labeled JP4-039 (BODIPY), (B) colocalization of JP4-039 (BODIPY) with Mitotracker, and (C) fluorescence over that in control animals for various body tissues after administration of JP4-039 (BODIPY).

FIG. 23. Effect of intraesophageal swallow of JP4-039 on survival in mice receiving (A) 29 Gy upper-body irradiation, (B) four daily fractions of 12 Gy irradiation, and (C) those with 3LL tumors that received 15 Gy upper-body irradiation.

FIG. 24. Survival of 32D c13 cells incubated in 10 μM JP4-039 for one hour prior to exposure to 0-8 Gy irradiation.

FIG. 25. (A) Percent of lung containing tumor following JP4-039 (BODIPY)+15 Gy thoracic irradiation or 15 Gy alone, (B) percent tumor cells positive for JP4-039 (BODIPY), and (C) Tumor cells in mice given intranasal adeno cre-recombinase prior to JP4-039 (BODIPY) in F15 alone (left), Gy thoracic-cavity irradiation (middle), or JP4-039 and 15 Gy (right).

FIG. 26. (A) JP4-039 (BODIPY-R6G) in F15 in esophageal SP population of GFP+ marrow chimeric mice 5 days after receiving 29 Gy upper-body irradiation, and (B) Immunohistochemical analysis of multilineage esophageal SP cell colony from single GFP+JP4-039 (BODIPY) in F15-treated mice.

FIG. 27. Emission spectra of GFP+, Mitotracker, and JP4-039 (BODIPY-R6G, and structure fluorochrome-labeled JP4-039 (BODIPY). Left trace, Fluorescence emission spectra of enhanced green fluorescent protein (EGFP) in pH 7 buffer. Center trace, Fluorescence emission spectra of MitoTracker 8 Deep Red FM in methanol. Right trace, Fluorescence emission spectra of BODIPY® R6G JP4-039 succinyl ester in methanol.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

As used herein, the term “patient” refers to members of the animal kingdom including but not limited to human beings and implies no relationship between a doctor or veterinarian and a patient. The term “reactive oxygen species” (“ROS”) includes, but is not limited to, superoxide anion, hydroxyl, and hydroperoxide radicals.

As used herein, the term ‘comprising’ is open-ended and may be synonymous with “including,” “containing,” or “characterized by”. The term ‘consisting essentially of’ limits the scope of a claim to the specified materials or steps and those that do not materially affect the basic and novel characteristic(s) of the claimed invention. The term ‘consisting of’ excludes any element, step, or ingredient not specified in the claim. As used herein, embodiments “comprising” one or more stated elements or steps also include, but are not limited to embodiments “consisting essentially of” and “consisting of” these stated elements or steps.

An antioxidant compound is defined herein as a compound that decreases the rate of oxidation of other compounds or prevents a substance from reacting with oxygen or oxygen containing compounds. A compound may be determined to be an antioxidant compound by assessing its ability to decrease molecular oxidation and/or cellular sequellae of oxidative stress, for example, and without limitation, the ability to decrease lipid peroxidation and/or decrease oxidative damage to protein or nucleic acid. In one embodiment, an antioxidant has a level of antioxidant activity between 0.01 and 1000 times the antioxidant activity of ascorbic acid in at least one assay that measures antioxidant activity.

Methods of preventing (substantially or completely preventing ionizing irradiation-induced esophagitis) or mitigating (reducing the symptoms, sequelae, etc. associated with ionizing irradiation-induced esophagitis) ionizing irradiation-induced esophagitis in a subject are provided. The methods comprise administering to the patient prior to, during or after exposure of the subject to radiation, a composition comprising an amount of a targeted nitroxide compound effective to prevent, mitigate or treat radiation injury in the subject. Targeted nitroxide compounds useful in these methods are described below.

Provided herein are compounds and compositions comprising a targeting group and a cargo, such as a nitroxide-containing group. The cargo may be any useful compound, such as an antioxidant, as are well known in the medical and chemical arts. The cargo may comprise a factor having anti-microbial activity. For example, the targeting groups may be cross-linked to antibacterial enzymes, such as lysozyme, or antibiotics, such as penicillin. Other methods for attaching the targeting groups to a cargo are well known in the art. In one embodiment, the cargo is an antioxidant, such as a nitroxide-containing group.

While the generation of ROS in small amounts is a typical byproduct of the cellular respiration pathway, certain conditions, including a disease or other medical condition, may occur in the patient when the amount of ROS is excessive to the point where natural enzyme mechanisms cannot scavenge the amount of ROS being produced. Therefore, compounds, compositions and methods that scavenge reactive oxygen species that are present within the mitochondrial membrane of the cell are useful and are provided herein. These compounds, compositions and methods have the utility of being able to scavenge an excess amount of ROS being produced that naturally occurring enzymes SOD and catalase, among others, cannot cope with.

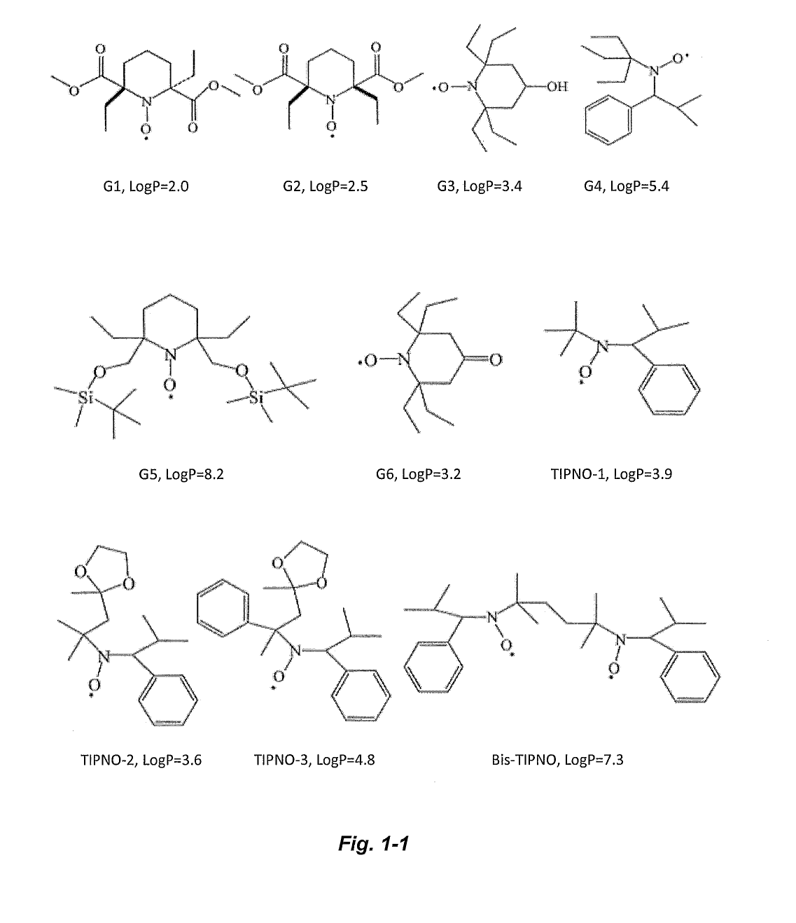

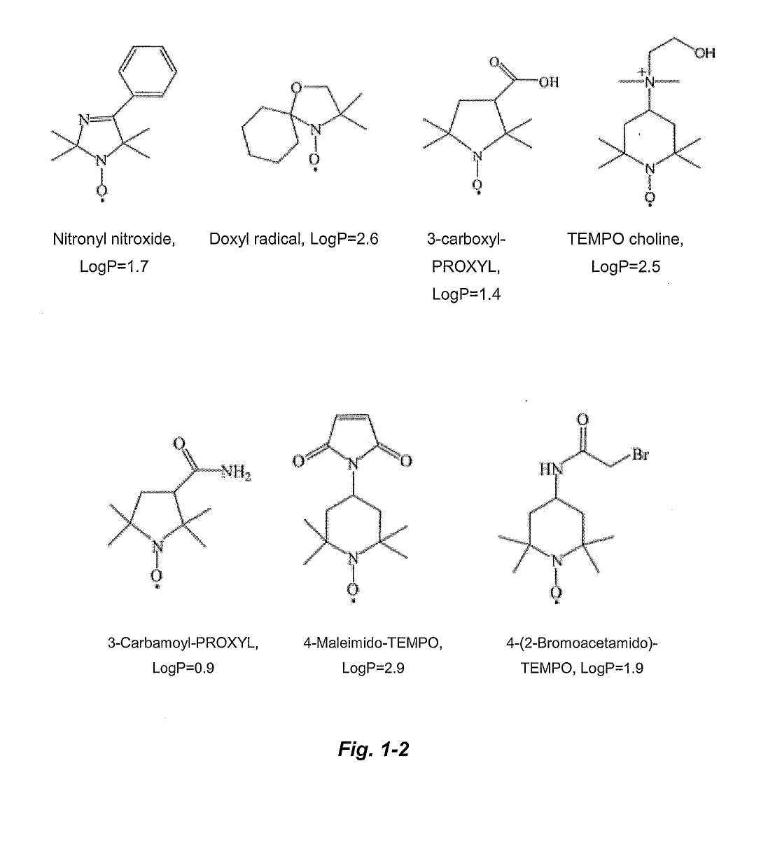

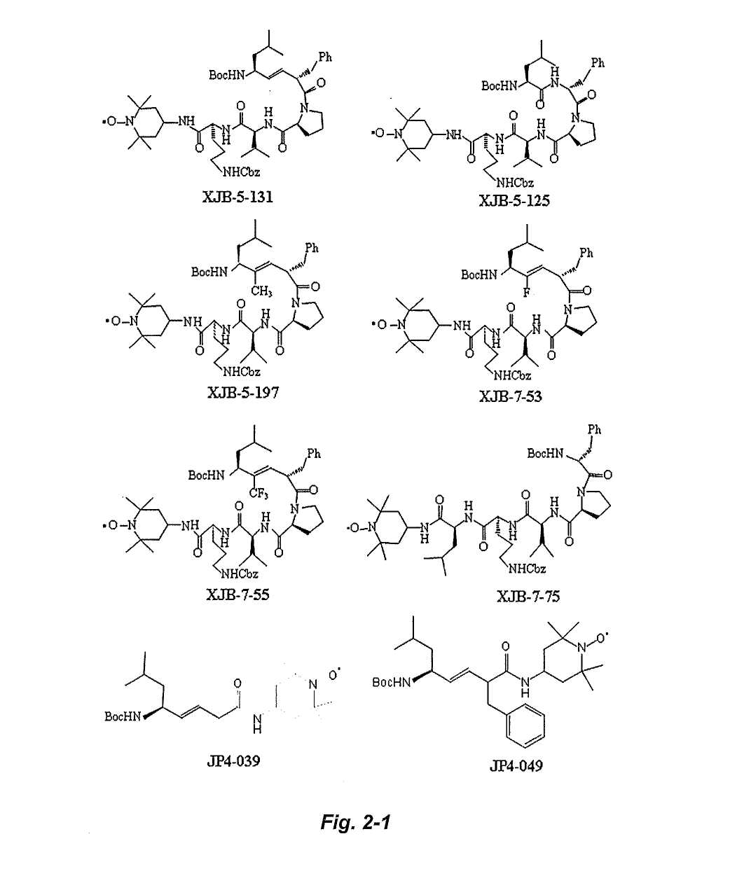

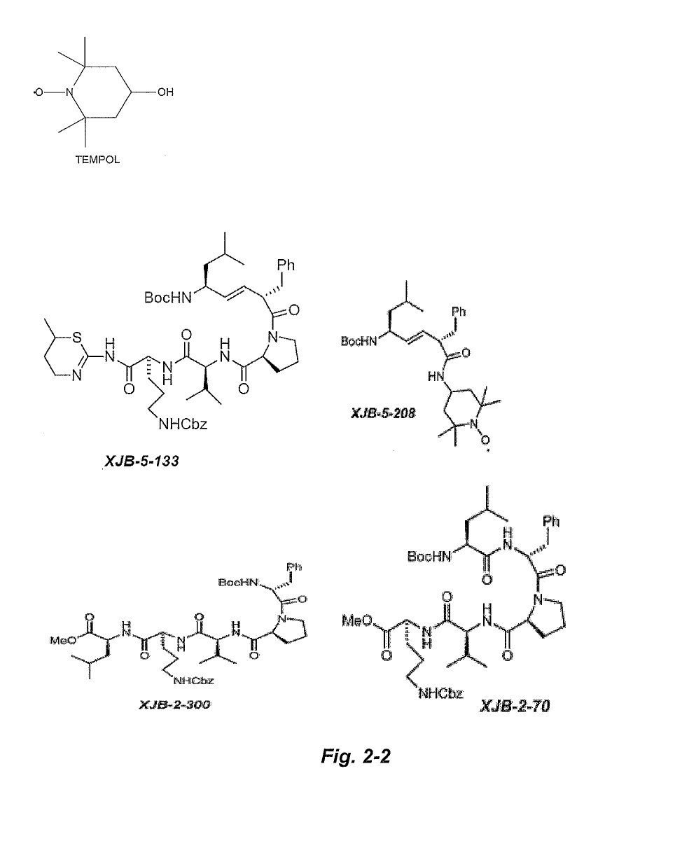

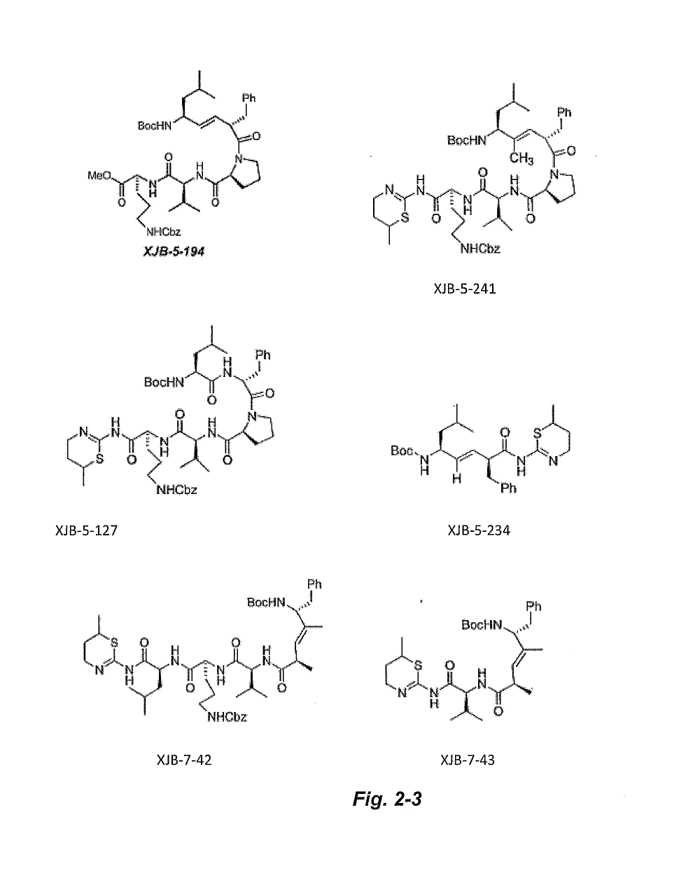

According to one embodiment, compounds useful in the methods and compositions described herein are disclosed in U.S. Pat. Nos. 7,718,603, 7,528,174, United States Patent Application Publication No. 2010/0035869 A1, and International (PCT) Patent Application Publication Nos. WO 2010/009389 A1 and WO 2010/009405 A2, each of which is incorporated herein by reference in its entirety for their disclosure of antioxidant compounds and compositions and their description of such compounds or compositions as being useful as mitochondria-targeting antioxidants. FIGS. 1 and 2 depict certain compounds from those publications.

In one non-limiting embodiment, the compound has the structure:

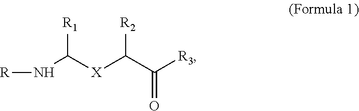

wherein X is one of

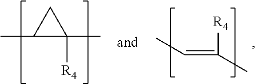

and R₁, R₂ and R₄ are, independently, hydrogen, C₁-C₆ straight or branched-chain alkyl, optionally including a phenyl(C₆H₅) group, that optionally is methyl-, hydroxyl-, chloro- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl. R₃ is —NH—R₅, —O—R₅ or —CH₂—R₅, where R₅ is an —N—O., —N—OH or N═O containing group. In one embodiment, R₃ is

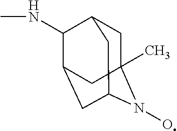

(1-Me-AZADO or 1-methyl 2-azaadamantane N-oxyl). In another embodiment R₃ is

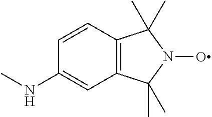

(TMIO; 1,1,3,3-tetramethylisoindolin-2-yloxyl).
R is —C(O)—R₆, —C(O)O—R₆, or —P(O)—(R₆)₂, wherein R₆ is C₁-C₆ straight or branched-chain alkyl optionally comprising one or more phenyl (—C₆H₅) groups, and that optionally are methyl-, ethyl-, hydroxyl-, chloro- or fluoro-substituted, including Ac (Acetyl, R=—C(O)—CH₃), Boc (R=—C(O)O-tert-butyl), Cbz (R=—C(O)O-benzyl(Bn)) groups. R also may be a diphenylphosphate group, that is,

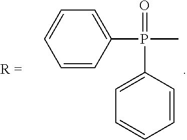

Excluded from this is the enantiomer XJB-5-208. In certain embodiments, R₁ is t-butyl and R₂ and R₄ are H; for instance:

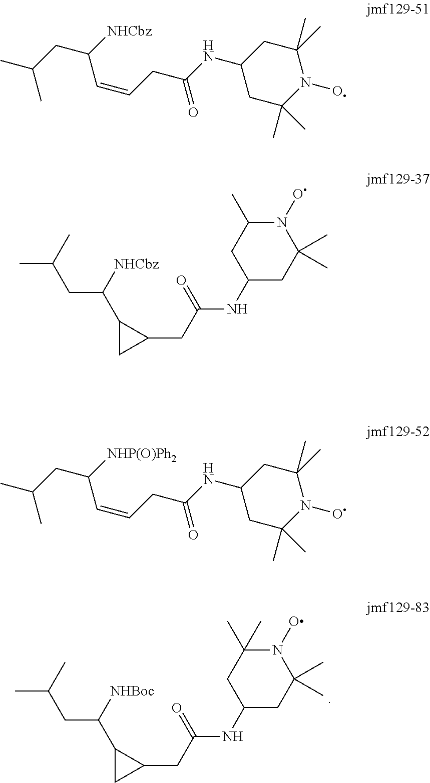

As used herein, unless indicated otherwise, for instance in a structure, all compounds and/or structures described herein comprise all possible stereoisomers, individually or mixtures thereof.

As indicated above, R₅ is an —N—O., —N—OH or —N═O containing group (not —N—O., —N—OH or —N═O alone, but groups containing those moieties, such as TEMPO, etc, as described herein). As is known to one ordinarily skilled in the art, nitroxide and nitroxide derivatives, including TEMPOL and associated TEMPO derivatives are stable radicals that can withstand biological environments. Therefore, the presence of the 4-amino-TEMPO (4-AT), TEMPOL or another nitroxide “payload” within the mitochondria membrane can serve as an effective and efficient electron scavenger of the ROS being produced within the membrane. Non-limiting examples of this include TEMPO (2,2,6,6-tetramethyl-4-piperidine 1-oxyl) and TEMPOL (4-hydroxy-TEMPO), in which, when incorporated into the compound described herein, for example, when R₃ is —NH—R₅, —O—R₅:

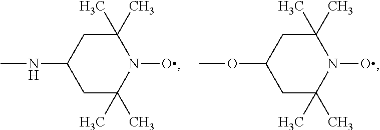

Additional non-limiting examples of —N—O., —N—OH or N═O containing group are provided below and in FIG. 1 (from Jiang, J., et al. “Structural Requirements for Optimized Delivery, Inhibition of Oxidative Stress, and Antiapoptotic Activity of Targeted Nitroxides”, J Pharmacol Exp Therap. 2007, 320(3):1050-60). A person of ordinary skill in the art would be able to conjugate (covalently attach) any of these compounds to the rest of the compound using common linkers and/or conjugation chemistries, such as the chemistries described herein. The listing below provides non-limiting excerpts from a list of over 300 identified commercially-available —N—O., —N—OH or N═O containing compounds that may be useful in preparation of the compounds or compositions described herein. The following are non-limiting examples of commercially-available —N—O., —N—OH or N═O containing groups that are expected to be useful in the compositions described herein (name, CAS No. (where known), excerpted from and with structures depicted in United States Patent Application Publication No. 2010/0035869 A1, and International (PCT) Patent Application Publication Nos. WO 2010/009389 A1 and WO 2010/009405): Trimethylamine N-Oxide, 1184-78-7; N,N-Dimethyldodecylamine N-Oxide, 1643-20-5, 70592-80-2; N-Benzoyl-N-Phenylhydroxylamine, 304-88-1; N,N-Diethylhydroxylamine, 3710-84-7; N,N-Dibenzylhydroxylamine, 14165-27-6, 621-07-8; Di-Tert-Butyl Nitroxide, 2406-25-9; N,N-Dimethylhydroxylamine Hydrochloride, 16645-06-0; Metobromuron, 3060-89-7; Benzyl-Di-Beta-Hydroxy Ethylamine-N-Oxide; Bis(Trifluoromethyl)Nitroxide, 2154-71-4; Triethylamine N-Oxide, 2687-45-8; N-Methoxy-N-Methylcarbamate, 6919-62-6; N,N-Bis(2-Chloro-6-Fluorobenzyl)-N-[(([2,2-Dichloro-1-(1,4-Thiazinan-4-yl)ethylidene]amino)carbonyl)oxy]amine; Tri-N-Octylamine N-Oxide, 13103-04-3; Diethyl(N-Methoxy-N-Methylcarbamoylmethyl)Phosphonate, 124931-12-0; N-Methoxy-N-Methyl-2-(Triphenylphosphoranylidene)Acetamide, 129986-67-0; N-Methoxy-N-Methyl-N′-[5-Oxo-2-(Trifluoromethyl)-5h-Chromeno[2,3-B]Pyridin-3-yl]Urea; N-[(4-Chlorobenzyl)Oxy]-N-([5-Oxo-2-Phenyl-1,3-Oxazol-4(5h)-yliden]methyl)acetamide; N-Methylfurohydroxamic Acid, 109531-96-6; N,N-Dimethylnonylamine N-Oxide, 2536-13-2; N-(Tert-Butoxycarbonyl)-L-Alanine N′-Methoxy-N′-Methylamide, 87694-49-3; 1-(4-Bromophenyl)-3-(Methyl([3-(Thfluoromethyl)Benzoyl]Oxy)Amino)-2-Propen-1-One; 2-([[(Anilinocarbonyl)Oxy](Methyl)Amino]Methylene)-5-(4-Chlorophenyl)-1,3-Cyclohexanedione; N-Methoxy-N-Methyl-2-(Trifluoromethyl)-1,8-Naphthyridine-3-Carboxamide; N-Methoxy-N-Methyl-Indole-6-Carboxamide; Desferrioxamin; AKOS 91254, 127408-31-5; N-[(3s,4r)-6-Cyano-3,4-Dihydro-3-Hydroxy-2,2-Dimethyl-2h-1-Benzopyran-4-yl]-N-Hydroxyacetamide, 127408-31-5; N-Methoxy-N-Methyl-1,2-Dihydro-4-Oxo-Pyrrolo[3,2,1-Ij]Quinoline-5-Carboxamide; Fr-900098; 2,2′-(Hydroxyimino)Bis-Ethanesulfonic Acid Disodium Salt, 133986-51-3; Fmoc-N-Ethyl-Hydroxylamine; Bis(N,N-Dimethylhydroxamido)Hydroxooxovanadate; Pyraclostrobin, 175013-18-0; 1-Boc-5-Chloro-3-(Methoxy-Methyl-Carbamoyl)Indazole; N-Methoxy-N-Methyl-Thiazole-2-Carboxamide; 4,4-Difluoro-N-Methyl-N-Methoxy-L-Prolinamide HCl; 3-Fluoro-4-(Methoxy(Methyl)Carbamoyl)Phenylboronic Acid, 913835-59-3; 1-Isopropyl-N-Methoxy-N-Methyl-1h-Benzo[D][1,2,3]Triazole-6-Carboxamide, 467235-06-9; (Trans)-2-(4-Chlorophenyl)-N-Methoxy-N-Methylcyclopropanecarboxamide; Bicyclo[2.2.1]Heptane-2-Carboxylic Acid Methoxy-Methyl-Amide; Akos Bc-0582; 3-(N,O-Dimethylhydroxylaminocarbonyl)Phenylboronic Acid, Pinacol Ester; and 1-Triisopropylsilanyl-1h-Pyrrolo[2,3-B]Pyridine-5-Carboxylic Acid Methoxy-Methyl-Amide.

According to one embodiment, the compound has the structure

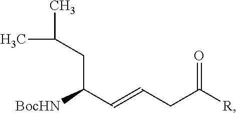

or the structure

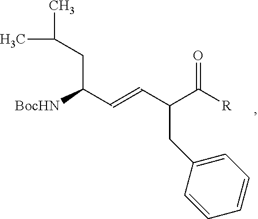

wherein R is —NH—R₁, —O—R₁ or —CH₂—R₁, and R₁ is an —N—O., —N—OH or N═O containing group. In one embodiment, R is —NH—R₁, and in another R is —NH-TEMPO.

According to another embodiment, the compound has the structure:

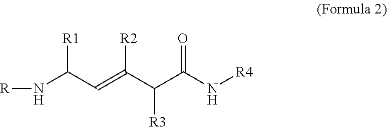

in which R1, R2 and R3 are, independently, hydrogen, C₁-C₆ straight or branched-chain alkyl, optionally including a phenyl(C₆H₅) group, that optionally is methyl-, hydroxyl-, chloro- or fluoro-substituted, including 2-methyl propyl, benzyl, methyl-, hydroxyl-, chloro- or fluoro-substituted benzyl, such as 4-hydroxybenzyl. R4 is an —N—O., —N—OH or N═O containing group. In one embodiment, R4 is

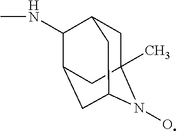

(1-Me-AZADO or 1-methyl 2-azaadamantane N-oxyl). In another embodiment R4 is

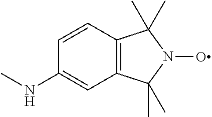

(TMIO; 1,1,3,3-tetramethylisoindolin-2-yloxyl). R is —C(O)—R5, —C(O)O—R5, or —P(O)—(R5)₂, wherein R5 is C₁-C₆ straight or branched-chain alkyl, optionally comprising one or more phenyl (—C₆H₅) groups, and that optionally are methyl-, ethyl-, hydroxyl-, chloro- or fluoro-substituted, including Ac, Boc, and Cbz groups. R also may be a diphenylphosphate group, that is,

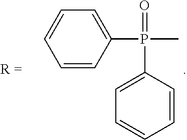

In certain specific embodiments, in which R4 is TEMPO, the compound has one of the structures A, A1, A2, or A3 (Ac=Acetyl=CH₃C(O)—):

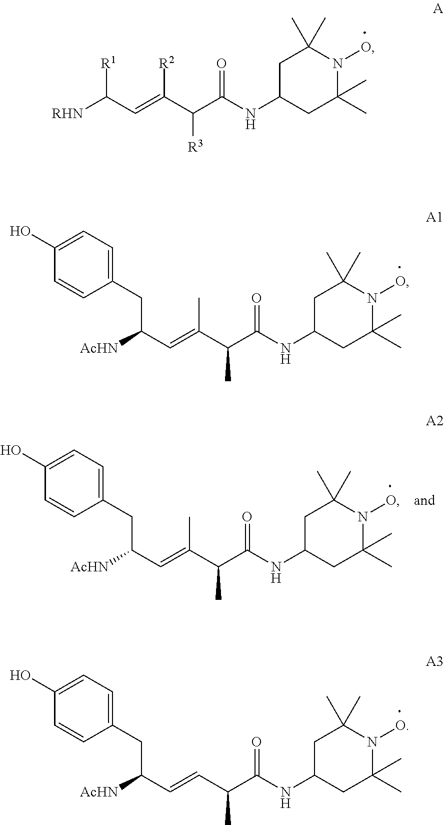

According to another embodiment, the compound has the structure

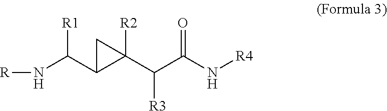

in which R1, R2 and R3 are, independently, hydrogen, C₁-C₆ straight or branched-chain alkyl, optionally including a phenyl(C₆H₅) group, that optionally is methyl-, hydroxyl-, chloro- or fluoro-substituted, including 2-methyl propyl, benzyl, methyl-, hydroxyl-, chloro- or fluoro-substituted benzyl, such as 4-hydroxybenzyl. R4 is an —N—O., —N—OH or N═O containing group. In one embodiment, R4 is

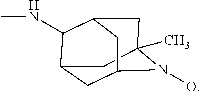

(1-Me-AZADO or 1-methyl 2-azaadamantane N-oxyl). In another embodiment R4 is

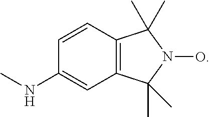

(TMIO; 1,1,3,3-tetramethylisoindolin-2-yloxyl). R is —C(O)—R5, —C(O)O—R5, or —P(O)—(R5)₂, wherein R5 is C₁-C₆ straight or branched-chain alkyl, optionally comprising one or more phenyl (—C₆H₅) groups, and that optionally are methyl-, ethyl-, hydroxyl-, chloro- or fluoro-substituted, including Ac, Hoc, and Cbz groups. R also may be a diphenylphosphate group, that is,

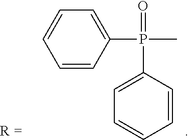

In certain specific embodiments, in which R4 is TEMPO, the compound has one of the structures D, D1, D2, or D3 (Ac=Acetyl=CH₃C(O)—):

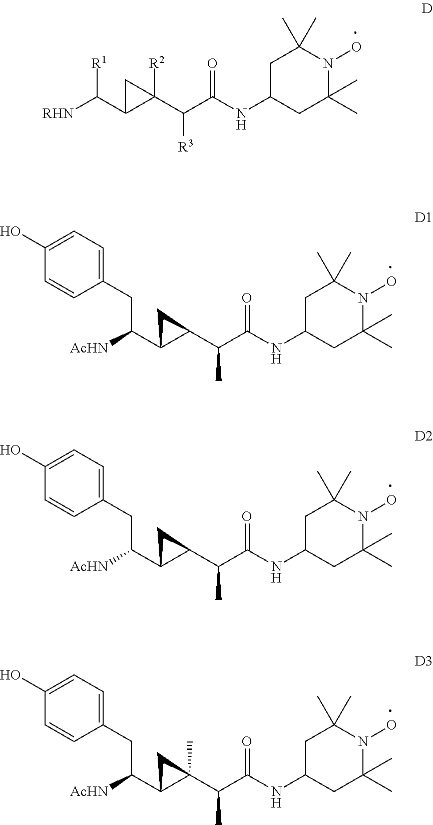

In another non-limiting embodiment, the compound has the structure:

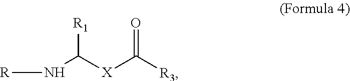

wherein X is one of and

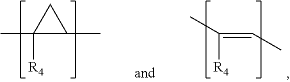

R₁ and R₄ are, independently, hydrogen, C₁-C₆ straight or branched-chain alkyl, optionally including a phenyl(C₆H₅) group, that optionally is methyl-, hydroxyl-, chloro- or fluoro-substituted, including: methyl, ethyl, propyl, 2-propyl, butyl, t-butyl, pentyl, hexyl, benzyl, hydroxybenzyl (e.g., 4-hydroxybenzyl), phenyl and hydroxyphenyl. R₃ is —NH—R₅, —O—R₅ or —CH₂—R₅, where R₅ is an —N—O., —N—OH or N═O containing group. In one embodiment, R₃ is

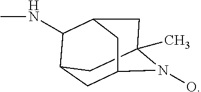

(1-Me-AZADO or 1-methyl azaadamantane N-oxyl). In another embodiment R₃ is

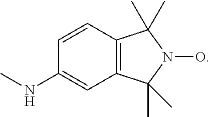

(TMIO; 1,1,3,3-tetramethylisoindolin-2-yloxyl).
R is —C(O)—R₆, —C(O)O—R₆, or —P(O)—(R₆)₂, wherein R₆ is C₁-C₆ straight or branched-chain alkyl optionally comprising one or more phenyl (—C₆H₅) groups, and that optionally are methyl-, ethyl-, hydroxyl-, chloro- or fluoro-substituted, including Ac (Acetyl, R=—C(O)—CH₃), Boc (R=—C(O)O-tert-butyl), Cbz (R=—C(O)O-benzyl(Bn)) groups. R also may be a diphenylphosphate group, that is,

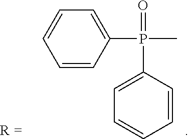

In one non-limiting embodiment, the compound has one of the structures

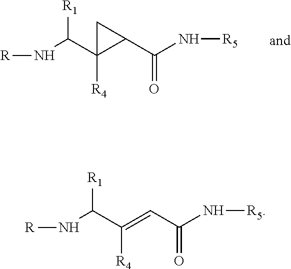

In yet another non-limiting embodiment, the compound has the structure

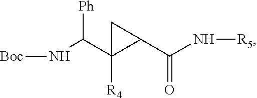

in which R₄ is hydrogen or methyl.

The compounds described above, such as the compound of Formula 1, can be synthesized by any useful method. The compound JP4-039 was synthesized by the method of Example 1. In one embodiment, a method of making a compound of Formula 1 is provided. The compounds are synthesized by the following steps:

reacting an aldehyde of structure R₁—C(O)—, wherein, for example and without limitation, R₁ is C₁-C₆ straight or branched-chain alkyl, optionally including a phenyl(C₆H₅) group, that optionally is methyl-, hydroxyl-, chloro- or fluoro-substituted, including including: methyl, ethyl, propyl, 2-propyl, butyl, t-butyl, pentyl, hexyl, benzyl, hydroxybenzyl (e.g., 4-hydroxybenzyl), phenyl and hydroxyphenyl, with (R)-2-methylpropane-2-sulfinamide to form an imine, for example

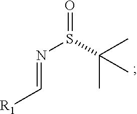

reacting a terminal alkyne-1-ol (HCC—R₂—CH₂—OH), wherein, for example and without limitation, R₂ is not present or is branched or straight-chained alkylene, including methyl, ethyl, propyl, etc., with a tert-butyl diphenylsilane salt to produce an alkyne, for example

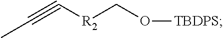

reacting (by hydrozirconation) the alkyne with the imine in the presence of an organozirconium catalyst to produce an alkene, for example

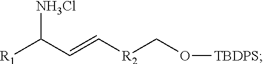

acylating the alkene to produce a carbamate, for example

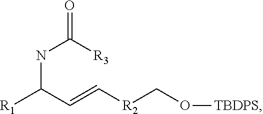

wherein, for example and without limitation, R₃ is C₁-C₆ straight or branched-chain alkyl, optionally including a phenyl(C₆H₅) group, that optionally is methyl-, hydroxyl-, chloro- or fluoro-substituted, including including: methyl, ethyl, propyl, 2-propyl, butyl, t-butyl, pentyl, hexyl, benzyl, hydroxybenzyl (e.g., 4-hydroxybenzyl), phenyl and hydroxyphenyl;
optionally, cyclopropanating the alkene and then acylating the alkene to produce a carbamate, for

### EXAMPLE

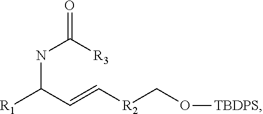

wherein, for example and without limitation, R₃ is C₁-C₆ straight or branched-chain alkyl, optionally including a phenyl(C₆H₅) group, that optionally is methyl-, hydroxyl-, chloro- or fluoro-substituted, including including: methyl, ethyl, propyl, 2-propyl, butyl, t-butyl, pentyl, hexyl, benzyl, hydroxybenzyl (e.g., 4-hydroxybenzyl), phenyl and hydroxyphenyl; removing the t-butyldiphenylsilyl group from the carbamate to produce an alcohol, for example

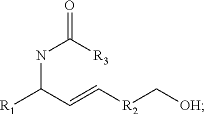

oxidizing the alcohol to produce a carboxylic acid, for example

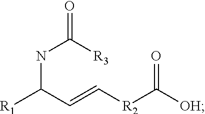

and reacting the carboxylic acid with a nitroxide-containing compound comprising one of a hydroxyl or amine in a condensation reaction to produce the antioxidant compound, for example

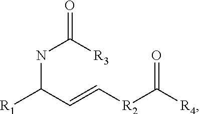

wherein R₄ is —NH—R₄ or —O—R₄, and R₄ is an —N—O., —N—OH or N═O containing group, such as described above.

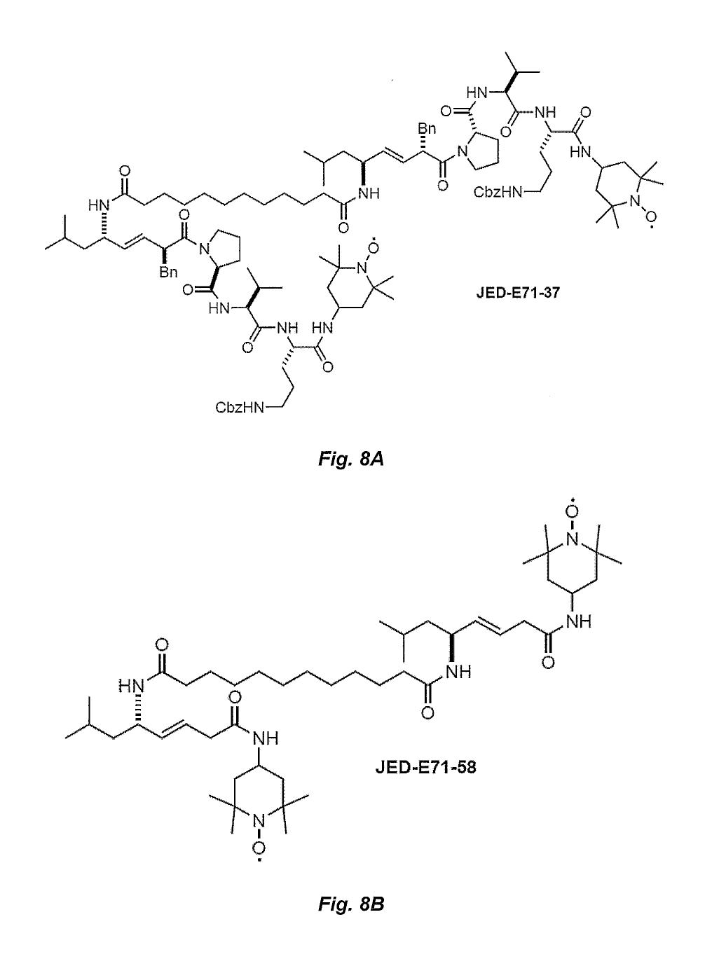

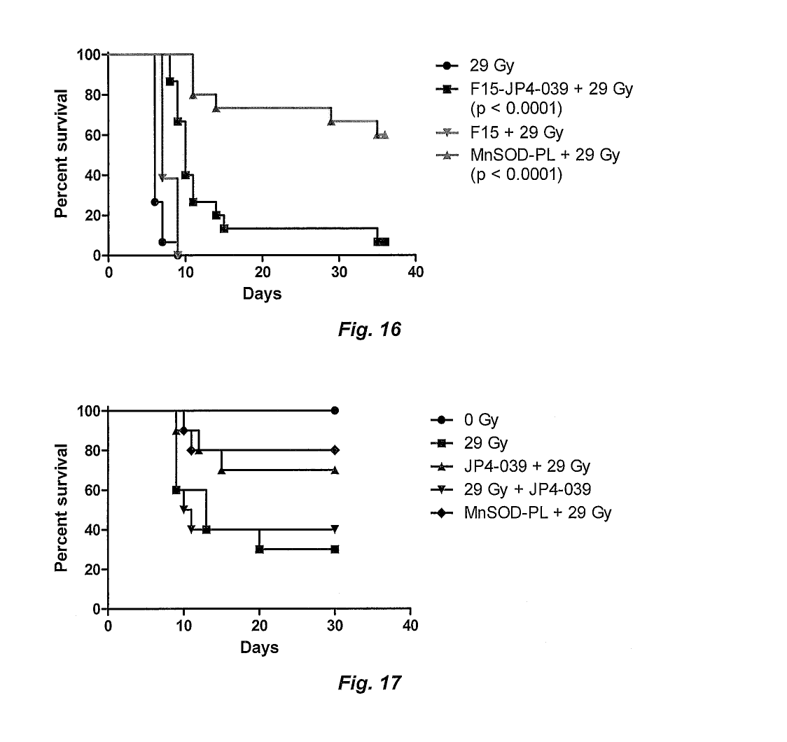

In another non-limiting embodiment, a compound is provided having the structure (i) R1-R2-R3 or (ii) R1, in which R1 and R3, when present, are a group having the structure —R4-R5, in which R4 is a mitochondria targeting group and R5 is —NH—R6, —O—R6 or —CH₂—R6, wherein R6 is an —N—O., N—OH or N═O containing group, such as TEMPO. R1 and R3 may be the same or different. Likewise, R4 and R5 for each of R1 and R3 may be the same or different. R2 is a linker that, in one non-limiting embodiment, is symmetrical. FIGS. 16A and 16B depicts two examples of such compounds. In one embodiment, R1 and R2 have the structure shown in formulas 1, 2, or 3, above, with all groups as defined above, including structures A, A1, A2 A3, D, D1, D2 and D3, above, an example of which is compound JED-E71-58, shown in FIG. 8B. In another embodiment, R1 and R2 are, independently, a gramicidin derivative, for example, as in the compound JED-E71-37, shown in FIG. 8A. Examples of gramicidin derivatives having an antioxidant cargo are provided herein, such as XJB-5-131 and XJB-5-125 (see, FIG. 2), and these compounds are further described both structurally and functionally in United States Patent Publication Nos. 20100035869, 20070161573 and 20070161544, U.S. Pat. Nos. 7,718,603, and 7,528,174, and International (PCT) Patent Application Publication Nos. WO 2010/009389 A1 and WO 2010/009405 A2, as well as in Jiang, J, et al. (Structural Requirements for Optimized Delivery, Inhibition of Oxidative Stress, and Antiapoptotic Activity of Targeted Nitroxides, J Pharmacol Exp Therap. 2007, 320(3):1050-60, see also, Hoye, A T et al., Targeting Mitochondria, Acc Chem Res. 2008, 41(1):87-97, see also, Wipf, P, et al., Mitochondrial Targeting of Selective Electron Scavengers: Synthesis and Biological Analysis of Hemigramicidin-TEMPO Conjugates, (2005) J Am Chem Soc. 2005, 127:12460-12461). Methods of making those compounds also are disclosed in those publications. The XJB compounds can be linked into a dimer, for example and without limitation, by reaction with the nitrogen of the BocHN groups (e.g., as in XJB-5-131), or with an amine, if present, for instance, if one or more amine groups of the compound is not acylated to form an amide (such as NHBoc or NHCbx).

In Jiang, J, et al. (J Pharmacol Exp Therap. 2007, 320(3):1050-60), using a model of ActD-induced apoptosis in mouse embryonic cells, the authors screened a library of nitroxides to explore structure-activity relationships between their antioxidant/antiapoptotic properties and chemical composition and three-dimensional (3D) structure. High hydrophobicity and effective mitochondrial integration were deemed necessary but not sufficient for high antiapoptotic/antioxidant activity of a nitroxide conjugate. By designing conformationally preorganized peptidyl nitroxide conjugates and characterizing their 3D structure experimentally (circular dichroism and NMR) and theoretically (molecular dynamics), they established that the presence of the β-turn/β-sheet secondary structure is essential for the desired activity. Monte Carlo simulations in model lipid membranes confirmed that the conservation of the D-Phe-Pro reverse turn in hemi-GS analogs ensures the specific positioning of the nitroxide moiety at the mitochondrial membrane interface and maximizes their protective effects. These insights into the structure-activity relationships of nitroxide-peptide and -peptide isostere conjugates are helpful in the development of new mechanism-based therapeutically effective agents, such as those described herein.

Targeting group R4 may be a membrane active peptide fragment derived from an antibiotic molecule that acts by targeting the bacterial cell wall. Examples of such antibiotics include: bacitracins, gramicidins, valinomycins, emiiatins, alamethicins, beauvericin, serratomolide, sporidesmolide, tyrocidins, polymyxins, monamycins, and lissoclinum peptides. The membrane-active peptide fragment derived from an antibiotic may include the complete antibiotic polypeptide, or portions thereof having membrane, and preferably mitochondria-targeting abilities, which is readily determined, for example, by cellular partitioning experiments using radiolabeled peptides. Examples of useful gramicidin-derived membrane active peptide fragments are the Leu-D-Phe-Pro-Val-Orn and D-Phe-Pro-Val-Orn-Leu hemigramicidin fragments. As gramicidin is cyclic, any hemigramicidin 5-mer is expected to be useful as a membrane active peptide fragment, including Leu-D-Phe-Pro-Val-Orn, D-Phe-Pro-Val-Orn-Leu, Pro-Val-Orn-Leu-D-Phe, Val-Orn-Leu-D-Phe-Pro and Orn-Leu-D-Phe-Pro-Val (from Gramicidin S). Any larger or smaller fragment of gramicidin, or even larger fragments containing repeated gramicidin sequences (e.g., Leu-D-Phe-Pro-Val-Orn-Leu-D-Phe-Pro-Val-Orn-Leu-D-Phe-Pro) are expected to be useful for membrane targeting, and can readily tested for such activity. In one embodiment, the Gramicidin S-derived peptide comprises a β-turn, which appears to confer to the peptide a high affinity for mitochondria. Derivatives of Gramicidin, or other antibiotic fragments, include isosteres (molecules or ions with the same number of atoms and the same number of valence electrons—as a result, they can exhibit similar pharmacokinetic and pharmacodynamic properties), such as (E)-alkene isosteres (see, United States Patent Publication Nos. 20070161573 and 20070161544 for exemplary synthesis methods). As with Gramicidin, the structure (amino acid sequence) of bacitracins, other gramicidins, valinomycins, enniatins, alamethicins, beauvericin, serratomolide, sporidesmolide, tyrocidins, polymyxins, monamycins, and lissoclinum peptides are all known, and fragments of these can be readily prepared and their membrane-targeting abilities can easily be confirmed by a person of ordinary skill in the art.

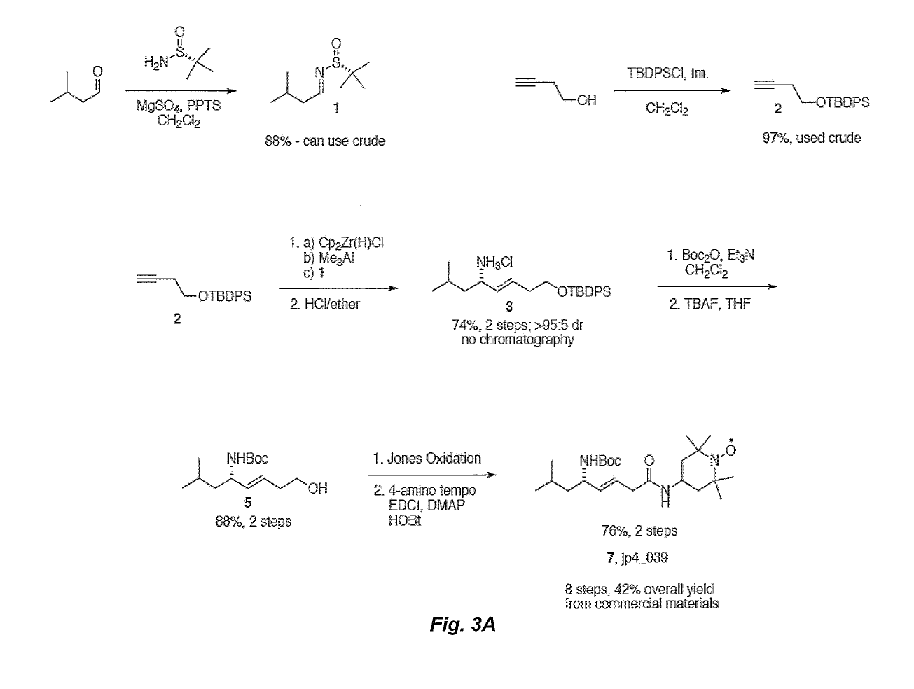

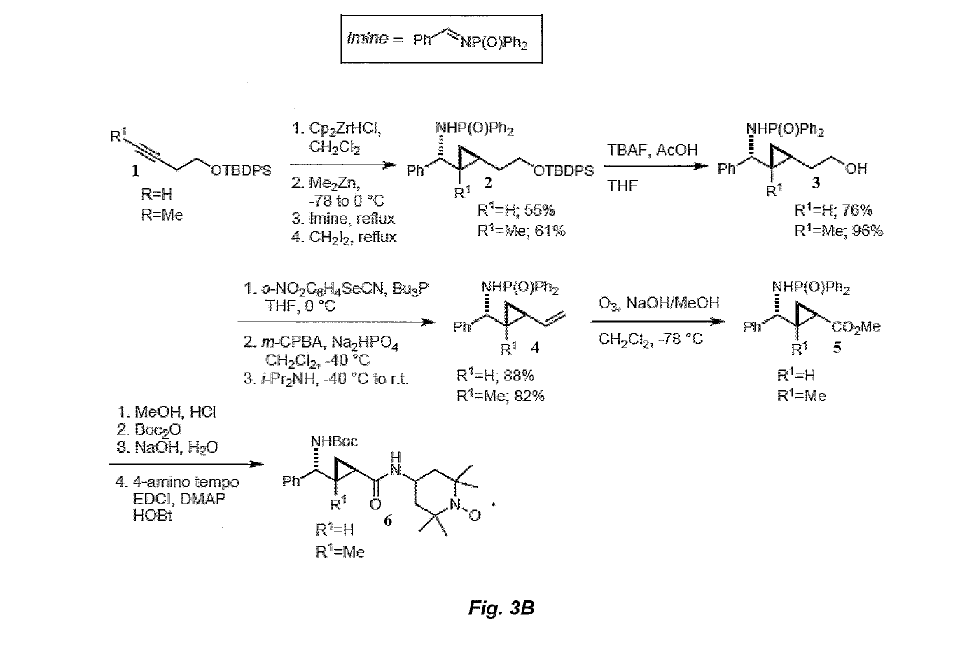

Alkene isosteres such as (E)-alkene isosteres of Gramicidin S (i.e., hemigramicidin) were used as part of the targeting sequence. See FIG. 3 for a synthetic pathway for (E)-alkene isosteres and reference number 2 for the corresponding chemical structure. First, hydrozirconation of alkyne (FIG. 3, compound 1) with Cp₂ZrHCl is followed by transmetalation to Me₂Zn and the addition of N-Boc-isovaleraldimine. The resulting compound (not shown) was then worked up using a solution of tetrabutylammonium fluoride (“TBAF”) and diethyl ether with a 74% yield. The resulting compound was then treated with acetic anhydride, triethylamine (TEA), and N,N-dimethylpyridin-4-amine (“DMAP”) to provide a mixture of diastereomeric allylic amides with a 94% yield which was separated by chromatography. Finally, the product was worked up with K₂CO₃ in methanol to yield the (E)-alkene, depicted as compound 2. The (E)-alkene, depicted as compound 2 of FIG. 3, was then oxidized in a multi-step process to yield the compound 3 (FIG. 3)—an example of the (E)-alkene isostere.

The compound 3 of FIG. 3 was then conjugated with the peptide H-Pro-Val-Om (Cbz)-OMe using 1-ethyl-3-(3-dimethylaminopropyl carbodiimide hydrochloride) (EDC) as a coupling agent. The peptide is an example of a suitable targeting sequence having affinity for the mitochondria of a cell. The resulting product is shown as compound 4a in FIG. 3. Saponification of compound 4a followed by coupling with 4-amino-TEMPO (4-AT) afforded the resulting conjugate shown as compound 5a in FIG. 3, in which the Leu-DPhe peptide bond has been replaced with an (E)-alkene.

In an alternate embodiment, conjugates 5b in FIG. 3 was prepared by saponification and coupling of the peptide 4b (Boc-Leu-DPhe-Pro-Val-Orn(Cbz)-OMe) with 4-AT. Similarly, conjugate 5c in FIG. 3 was prepared by coupling the (E)-alkene isostere as indicated as compound 3 in FIG. 3 with 4-AT. These peptide and peptide analogs are additional examples of suitable targeting sequences having an affinity to the mitochondria of a cell.

In another embodiment, peptide isosteres may be employed as the conjugate. Among the suitable peptide isosteres are trisubstituted (E)-alkene peptide isosteres and cyclopropane peptide isosteres, as well as all imine addition products of hydro- or carbometalated internal and terminal alkynes for the synthesis of d-i and trisubstituted (E)-alkene and cyclopropane peptide isosteres. See Wipf et al. Imine additions of internal alkynes for the synthesis of trisubstituted (E)-alkene and cyclopropane isosteres, Adv Synth Catal. 2005, 347:1605-1613. These peptide mimetics have been found to act as β-turn promoters. See Wipf et al. Convergent Approach to (E)-Alkene and Cyclopropane Peptide Isosteres, Org Lett. 2005, 7(1):103-106.

The linker, R2, may be any useful linker, chosen for its active groups, e.g., carboxyl, alkoxyl, amino, sulihydryl, amide, etc. Typically, aside from the active groups, the remainder is non-reactive (such as saturated alkyl or phenyl), and does not interfere, sterically or by any other physical or chemical attribute, such as polarity or hydrophobicity/hydrophilicity, in a negative (loss of function) capacity with the activity of the overall compound. In one embodiment, aside from the active groups, the linker comprises a linear or branched saturated C₄-C₂₀ alkyl. In one embodiment, the linker, R2 has the structure

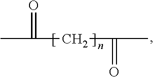

in which n is 4-18, including all integers therebetween, in one embodiment, 8-12, and in another embodiment, 10.

A person skilled in the organic synthesis arts can synthesize these compounds by crosslinking groups R1 and R3 by any of the many chemistries available. In one embodiment, R1 and R3 are to R2 by an amide linkage (peptide bond) formed by dehydration synthesis (condensation) of terminal carboxyl groups on the linker and an amine on R1 and R3 (or vice versa). In one embodiment, R1 and R3 are identical or different and are selected from the group consisting of: XJB-5-131, XJB-5-125, XJB-7-75, XJB-2-70, XJB-2-300, XJB-5-208, XJB-5-197, XJB-5-194, JP4-039 and JP4-049, attached in the manner shown in FIGS. 8A and 8B.

In a therapeutic embodiment, a method of preventing or mitigating radiation-induced esophagitis a patient (e.g., a patient in need of treatment with a free-radical scavenger) is provided, comprising administering to the subject an amount of one or more nitroxide or cell-cycle arresting compounds described herein. As described above, a number of diseases, conditions or injuries can be ameliorated or otherwise treated or prevented by administration of free radical scavenging compounds, such as those described herein.

In any case, as used herein, any compound (e.g., active agent(s), composition(s), etc.) used for prevention or mitigation in a patient of injury, e.g. esophagitis, caused by radiation exposure is administered in an amount effective to prevent or mitigate such injury, namely in an amount and in a dosage regimen effective to prevent injury or to reduce the duration and/or severity of the injury resulting from radiation exposure. According to one non-limiting embodiment, an effective dose of a compound described herein ranges from 0.1 or 1 mg/kg to 100 mg/kg, including any increment or range therebetween, including 1 mg/kg, 5 mg/kg, 10 mg/kg, 20 mg/kg, 25 mg/kg, 50 mg/kg, and 75 mg/kg. Effective doses may also be expressed in terms of the concentration within the specific formulation, including the range from 0.1 to 100 mg/ml. Further dosage range may be expressed in total weight of active agent, including the range from 1 microgram to 100 mg. However, for each compound described herein, an effective dose or dose range is expected to vary from that of other compounds described herein for any number of reasons, including the molecular weight of the compound, bioavailability, specific activity, etc. For example and without limitation, where XJB-5-131 is the antioxidant, the dose may be between about 0.1 and 20 mg/kg, or between about 0.3 and 10 mg/kg, or between about 2 and 8 mg/kg, or about 2 mg/kg and where either JP4-039, JED-E71-37 or JED-E71-58 is the antioxidant, the dose may be between about 0.01 and 50 mg/kg, or between about 0.1 and 20 mg/kg, or between about 0.3 and 10 mg/kg, or between about 2 and 8 mg/kg, or about 2 mg/kg, or between 4 and 8 mg/ml, or between 1 microgram and 10 mg. The therapeutic window between the minimally-effective dose, and maximum tolerable dose in a subject can be determined empirically by a person of skill in the art, with end points being determinable by in vitro and in vivo assays, such as those described herein and/or are acceptable in the pharmaceutical and medical arts for obtaining such information regarding radioprotective agents. Different concentrations of the agents described herein are expected to achieve similar results, with the drug product administered, for example and without limitation, once prior to an expected radiation dose, such as prior to radiation therapy or diagnostic exposure to ionizing radiation, during exposure to radiation, or after exposure in any effective dosage regimen. The compounds can be administered orally one or more times daily, once every two, three, four, five or more days, weekly, monthly, etc., including increments therebetween. A person of ordinary skill in the pharmaceutical and medical arts will appreciate that it will be a matter of simple design choice and optimization to identify a suitable dosage regimen for prevention, mitigation or treatment of injury due to exposure to radiation.

The compounds described herein also are useful in preventing or mitigating (to make less severe) injury, such as esophagitis caused by radiation exposure. By “radiation,” in the context of this disclosure, it is meant types of radiation that result in the generation of free radicals, e.g., reactive oxygen species (ROS), as described herein. The free radicals are produced, for example and without limitation, by direct action of the radiation, as a physiological response to the radiation and/or as a consequence of damage/injury caused by the radiation. In one embodiment, the radiation is ionizing radiation. Ionizing radiation consists of highly-energetic particles or waves that can detach (ionize) at least one electron from an atom or molecule. Examples of ionizing radiation are energetic beta particles, neutrons, and alpha particles. The ability of light waves (photons) to ionize an atom or molecule varies across the electromagnetic spectrum. X-rays and gamma rays can ionize almost any molecule or atom; far ultraviolet light can ionize many atoms and molecules; near ultraviolet and visible light are ionizing to very few molecules. Microwaves and radio waves typically are considered to be non-ionizing radiation, though damage caused by, e.g., microwaves, may result in the production of free-radicals as part of the injury and/or physiological response to the injury.

The compounds typically are administered in an amount and dosage regimen to prevent, mitigate or treat the effects of exposure of a subject to radiation, for example to prevent or mitigate ionizing radiation-induced esophagitis. The compounds may be administered in any manner that is effective to treat, mitigate or prevent damage caused by the radiation. Examples of delivery routes include, without limitation: topical, for example, epicutaneous, inhalational, enema, ocular, otic and intranasal delivery; enteral, for example, orally, by gastric feeding tube or swallowing, and rectally; and parenteral, such as, intravenous, intraarterial, intramuscular, intracardiac, subcutaneous, intraosseous, intradermal, intrathecal, intraperitoneal, transdermal, iontophoretic, transmucosal, epidural and intravitreal, with oral approaches being preferred for prevention or mitigation of ionizing radiation-induced esophagitis. In a nonlimiting embodiment, the compound useful for mitigating or preventing radiation-induced esophagitis is swallowed in a novel liposomal formulation, described herein.

Therapeutic/pharmaceutical compositions are prepared in accordance with acceptable pharmaceutical procedures, such as described in Remington: The science and Practice of Pharmacy, 21st edition, ed. Paul Beringer et al., Lippincott, Williams & Wilkins, Baltimore, Md. Easton, Pa. (2005) (see, e.g., Chapter 39, pp. 745-775 for examples of liquid formulations and methods of making such formulations).

The compounds described herein may be compounded or otherwise manufactured into a suitable composition for use, such as a pharmaceutical dosage form or drug product in which the compound is an active ingredient. The drug product described herein is an oral liquid that delivers the drug agent to the esophagus of a patient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

According to one non-limiting embodiment, the formulation is a liposome or multiphase (a liquid comprising more than one phase, such as oil in water, water in oil, liposomes or multi-lamellar structures) composition comprising a phospholipid, a non-ionic detergent, and a cationic lipid, such as a composition comprising a phosphatidyl choline, a non-ionic surfactant, and a quaternary ammonium salt of a lipid-substituted D or L glutamic acid or aspartic acid, and an aqueous solvent. The liposomes or multiphase liquids and the ingredients thereof are pharmaceutically acceptable. They are typically formulated using an aqueous solvent, such as water, normal saline or PBS.

Phospholipids include any natural or synthetic diacylglyceryl phospholiopids (such as phosphatidyl choline, phosphotidylethanolamine, phosphotidylserine, phosphatidylinositol, phosphatidylinositol phosphate, etc) and phosphosphingolipids that is capable of forming self-assembly liposomes. In one example the phospolipid is a phosphatidyl choline, a compound that comprises a choline head group, glycerophosphoric acid and fatty acid. Phosphatidyl choline can be obtained from eggs, soy or any suitable source and can be synthesized.

A nonionic surfactant, is a surfactant containing no charged groups. Nonionic surfactants comprise a hydrophilic head group and a lipophilic tail group, such as a single- or double-lipophilic chain surfactant. Examples of lipophilic tail groups include lipophilic saturated or unsaturated alkyl groups (fatty acid groups), steroidal groups, such as cholesteryl, and vitamin E (e.g., tocopheryl) groups, such as a polysorbate (a polyoxyethylene sorbitan), for example Tween 20, 40, 60 or 80. More broadly, non-ionic surfactants include: glyceryl esters, including mono-, di- and tri-glycerides; fatty alcohols; and fatty acid esters of fatty alcohols or other alcohols, such as propylene glycol, polyethylene glycol, sorbitan, sucrose and cholesterol.

A cationic lipid is a compound having a cationic head and a lipophilic tail. Included are cationic lipids that are quaternary ammonium salts, such as quaternary ammonium salts of lipid-substituted D and L glutamic acid or aspartic acid, such as glutarnic acid dialkyl amides, including for example L-glutamic acid-1,5,-dioleyl amide. Other commercially-available examples of cationic lipids (e.g., available from Avanti Polar Lipids) include DC-Cholesterol (3β-[N—(N′,N′-dimethylaminoethane)-carbamoyl]cholesterol hydrochloride), DOTAP (e.g., 1,2-dioleoyl-3-trimethylammonium-propane (chloride salt)), DODAP (e.g., 1,2-dioleoyl-3-dimethylammonium-propane), DDAB (e.g., Dimethyldioctadecylammonium (Bromide Salt)), ethyl-PC (e.g., 1,2-dilauroyl-sn-glycero-3-ethylphosphocholine (chloride salt)) and DOTMA (e.g., 1,2-di-O-octadecenyl-3-trimethylammonium propane (chloride salt)).

The ratio of ingredients (phospholipid:nonionic surfactant:cationic lipid) can vary greatly, so long as a useful multilamellar structure is obtained that is able to deliver the active agents described herein. Further, each different combination of ingredients might have different optimal ratios. The ability to determine optimal ratios does not require undue experimentation because the ability of any formulation to deliver the active agent is readily tested as described herein, and as is generally known in the pharmaceutical arts. Liposome and multilamellar structures are common delivery vehicles for active agents and their manufacture, physical testing and biological assays to determine effectiveness are well-known. In the example below, the phospholipid:nonionic surfactant:cationic lipid ratio is 4:1:1 w/w (soy PC:Tween-80:N,N-di oleylamine amido-L-glutamate). Useful phospholipid:nonionic surfactant:cationic lipid ratios include, for example: from 0.1-10:0.1-10:0.1-10 (w/w), and in certain instances the nonionic surfactant:cationic lipid (w/w) ratio is approximately the same and/or the phospholipid constituent is from 2 to 10 times (w/w) that of the nonionic surfactant and cationic lipid.

In a nonlimiting embodiment, the formulation has a composition comprising soy phosphatidyl choline, Tween-80, and N,N-dioleylamine amido-L-glutamate in a ratio of 4:1:1 w/w, termed F15. In a further nonlimiting embodiment, the formulation may be cationically charged to facilitate adherence to the esophageal mucosa as the formulation containing the targeted nitroxide is swallowed.

The compounds described herein are administered in an amount effective to prevent or mitigate ionizing radiation-induced esophagitis. As one of ordinary skill in the pharmaceutical or medical arts would recognize, each different compound would have a specific activity in this use and the bioavailability of the compound would depend on the dosage form, with certain formulations rendering higher specific activity that other formulations with the same active compound. Based on the present disclosure, one of ordinary skill also would be able to optimize the formulation to best protect a patient against esophagitis. As the “patient” may be human or a mammal, such as a dog in a veterinary setting, different formulations may have different specific activities in each species, and optimal formulations can be prepared for each case. In the Examples below, in the F15 formulation, the concentration of JP4-039 was 8 mg/mL. Effective ranges in the formulation include from 0.1 to 100 mg/mL, from 0.5 to 10 mg/mL, from 0.1 to 100 mg/kg in the subject or from 0.5 to 10 mg/kg in the subject.

### Example 1

### Synthesis of JP4-039 (see FIG. 3)

Synthesis of JP4-039 was accomplished according to the following.

### (R,E)-2-Methyl-N-(3-methylbutylidene)propane-2-sulfinamide (1)

(Staas, D. D.; Savage, K. L.; Homnick, C. F.; Tsou, N.; Ball, R. G. J. Org. Chem., 2002, 67, 8276)—To a solution of isovaleraldehyde (3-Methylbutyraldehyde, 5.41 mL, 48.5 mmol) in CH₂Cl₂ (250 mL) was added (R)-2-methylpropane-2-sulfinamide (5.00 g, 40.4 mmol), MgSO₄ (5.0 eq, 24.3 g, 202 mmol) and PPTS (10 mol %, 1.05 g, 4.04 mmol) and the resulting suspension was stirred at RT (room temperature, approximately 25° C.) for 24 h. The reaction was filtered through a pad of Celite® and the crude residue was purified by chromatography on SiO₂ (3:7, EtOAc:hexanes) to yield 6.75 g (88%) as a colorless oil. ¹H NMR δ 8.07 (t, 1H, J=5.2 Hz), 2.47-2.38 (m, 2H), 2.18-1.90 (m, 1H), 1.21 (s, 9H), 1.00 (d, 6H, J=6.7 Hz). As an alternative, filtration through a pad of SiO₂ provides crude imine that functions equally well in subsequent reactions.

### (But-3-ynyloxy)(tert-butyl)diphenylsilane (2)

(Nicolaou, K. C. et al. J. Am. Chem. Soc. 2006, 128, 4460)—To a solution of 3-butyn-1-ol (5.00 g, 71.3 mmol) in CH₂Cl₂ (400 mL) was added imidazole (5.40 g, 78.5 mmol) and TBDPSCl ((tert-butyl)diphenylsilane chloride) (22.0 g, 78.5 mmol) and the reaction was stirred at RT for 22 h. The reaction was filtered through a pad a SiO₂, the SiO₂ washed with CH₂Cl₂ and the colorless solution concentrated to yield 21.4 g (97%) of crude alkyne that was carried on without further purification.

### (S,E)-8-(tert-Butyldiphenylsilyloxy)-2-methyloct-5-en-4-amine hydrochloride (3)

To a solution of (2) (15.9 g, 51.5 mmol) in CH₂Cl₂ (300 mL) was added zirconocene hydrochloride (15.1 g, 58.4 mmol) in 3 portions and the resulting suspension was stirred at RT for 10 min. The resulting yellow solution was cooled to 0° C. and Me₃Al (2.0 M in hexanes, 27.5 mL, 54.9 mmol) was added and stirred for 5 minutes followed by addition of a solution of imine (1) (6.50 g, 34.3 mmol) in CH₂Cl₂ (50 mL) and the orange solution was stirred for an additional 4 h while allowed to warm to rt. The reaction was quenched with MeOH, diluted with H₂O and CH₂Cl₂ and HCl (1 M) was added to break up the emulsion (prolonged stirring with Rochelle's salt can also be utilized). The organic layer was separated and the aqueous layer was washed with CH₂Cl₂ (2×). The organic layers were combined, washed with brine, dried (MgSO₄), filtered though a pad of Celite® and concentrated. Since the crude oil was contaminated with metal salts, the oil was dissolved in Et2O (diethyl ether, Et=ethyl), allowed to sit for 2 h, and then filtered though a pad of Celite® and concentrated. Analysis of the crude residue by 1H NMR showed only 1 diastereomer (>95:5 dr).

To the crude residue in Et₂O (800 mL) was added HCl (4.0 M in dioxane, 17.2 mL, 68.7 mmol) and the reaction was stirred for 30 minutes, during which time a white precipitate formed. The precipitate was filtered, washed with dry Et₂O, and dried to afford 11.0 g (74% over 2 steps) of (3) as a colorless solid. mp 151-154° C.; [α]_{D} −2.9 (c 1.0, CH₂Cl₂); ¹H NMR δ 8.42 (bs, 3H), 7.70-7.55 (m, 4H), 7.48-7.30 (m, 6H), 5.90 (dt, 1H, J=14.9, 7.5 Hz), 5.52 (dd, 1H, J=15.4, 8.4 Hz), 3.69 (appt, 3H, J=6.5 Hz), 2.45-2.20 (m, 2H), 1.80-1.50 (m, 3H), 1.03 (s, 9H), 0.95-0.84 (m, 6H); ¹³C NMR δ 135.5, 134.5, 133.7, 129.5, 127.6, 127.3, 63.0, 52.9, 42.1, 35.6, 26.7, 24.4, 22.9, 21.5, 19.1; EIMS m/z 395 ([M−HCl]⁺, 40), 338 (86), 198 (100); HRMS (EI) m/z calcd for C₂₅H₃₇NOSi (M−HCl) 395.2644, found 395.2640.

### (S,E)-tert-Butyl 8-(tert-butyldiphenylsilyloxy)-2-methyloct-5-en-4-ylcarbamate (4)

To a solution of (3) (10.5 g, 24.3 mmol) in CH₂Cl₂ (400 mL) was added Et₃N (triethylamine) (3.0 eq, 10.3 mL, 72.9 mmol) and Boc₂O (1.05 eq, 5.74 g, 25.5 mmol) and the resulting suspension was stirred at RT for 14 h. The reaction was quenched with sat. aq. NH₄Cl, the organic layers separated, dried (MgSO₄), filtered and concentrated. The crude residue was carried onto the next step without further purification.

### (S,E)-tert-Butyl 8-hydroxy-2-methyloct-5-en-4-ylcarbamate (5)

To a solution of crude (4) (12.0 g, 24.3 mmol) in THF (200 mL) at 0° C. was added TBAF (1.0 M in THF, 1.25 eq, 30.4 mL, 30.4 mmol) and the reaction was warmed to RT and stirred for 2 h. The reaction was quenched with sat. aq. NH₄Cl, organic layer washed with brine, dried (MgSO₄), filtered and concentrated. The crude residue was purified by chromatography on SiO₂ (3:7, EtOAc:hexanes) to yield 5.51 g (88%, 2 steps) as a colorless oil. [α]_{D} −12.7 (c 1.0, CH₂Cl₂); ¹H NMR δ 5.56 (dt, 1H, J=15.3, 6.9 Hz), 5.41 (dd, 1H, J=15.4, 6.4 Hz), 4.41 (bs, 1H), 4.06 (bin, 1H), 3.65 (appbq, 2H, J=5.7 Hz), 2.29 (q, 2H, J=6.3 Hz), 1.76 (bs, 1H), 1.68 (m, 1H), 1.44 (s, 9H), 1.33 (m, 2H), 0.92 (m, 6H); ¹³C NMR δ 155.4, 134.3, 126.9, 79.2, 61.5, 50.9, 44.5, 35.6, 28.3, 24.6, 22.5; EIMS in/z 257 ([M]⁺, 10), 227 (55), 171 (65); HRMS (EI) m/z calcd for C₁₄H₂₇NO₃ 257.1991. found 257.1994.

### (S,E)-5-(tert-Butoxycarbonylamino)-7-methyloct-3-enoic acid (6)

To a solution of (5) (1.00 g, 3.89 mmol) in acetone (40 mL) at 0° C. was added a freshly prepared solution of Jones Reagent (2.5 M, 3.89 mL, 9.71 mmol) and the reaction was stirred at 0° C. for 1 h. The dark solution was extracted with Et₂O (3×50 mL), the organic layers washed with water (2×75 mL), brine (1×50 mL), dried (Na₂SO₄), filtered and concentrated to yield 990 mg (94% crude) of acid (6) as a yellow oil that was used without further purification.

### TEMPO-4-yl-(S,E)-5-(tert-butoxycarbonylamino)-7-methyloct-3-enamide (7)

To a solution of (6) (678 mg, 2.50 mmol, crude) in CH₂Cl₂ (35 mL) at 0° C. was added 4-amino tempo (1.5 eq, 662 mg, 3.75 mmol), EDCI (1.2 eq, 575 mg, 3.00 mmol), DMAP (1.1 eq, 339 mg, 2.75 mmol) and HOBt-hydrate (1.1 eq, 377 mg, 2.75 mmol) and the resulting orange solution was stirred at RT for 14 h. The reaction was diluted with CH₂Cl₂, washed with sat. aq. NH₄Cl and the organic layer dried (Na₂SO₄), filtered and concentrated. The crude residue was purified by chromatography on SiO₂ (1:1 to 2:1, EtOAc/hexanes) to yield 857 mg (76%, 2 steps) as a peach colored solid. mp 61° C. (softening point: 51° C.); [α]_{D}²³+35.6 (c 0.5, DCM); ESIMS m/z 365 (40), 391 (50), 447 ([M+Na]⁺, 100), 257 (20); HRMS (ESI) m/z calcd for C₂₃H₄₂N₃O₄Na 447.3073. found 447.3109.

The compounds shown as Formula 4, above can be synthesized as shown in FIG. 3B. Briefly, synthesis was accomplished as follows: To a solution of compound (1) in CH₂Cl₂ was added zirconocene hydrochloride, followed by addition of Me₂Zn, then a solution of N-diphenylphosphoryl-1-phenylmethanimine (Imine). The reaction mixture was refluxed, filtered, washed, and dried to afford (2). Cleavage of the TBDPS protecting group was achieved by treating (2) with TBAF, which resulted in the formation of (3). The terminal alcohol (3) was dehydrated to alkene (4), which was further treated by ozonolysis to afford ester (5). Protocols similar to that given for the synthesis of JP4-039, above, were used to acylate the amino group with the Boc protecting group and to react the terminal carboxylic acid with 4-amino-TEMPO to afford (6).

### Example 2

### Testing of the Radioprotective Abilities of JP4-039

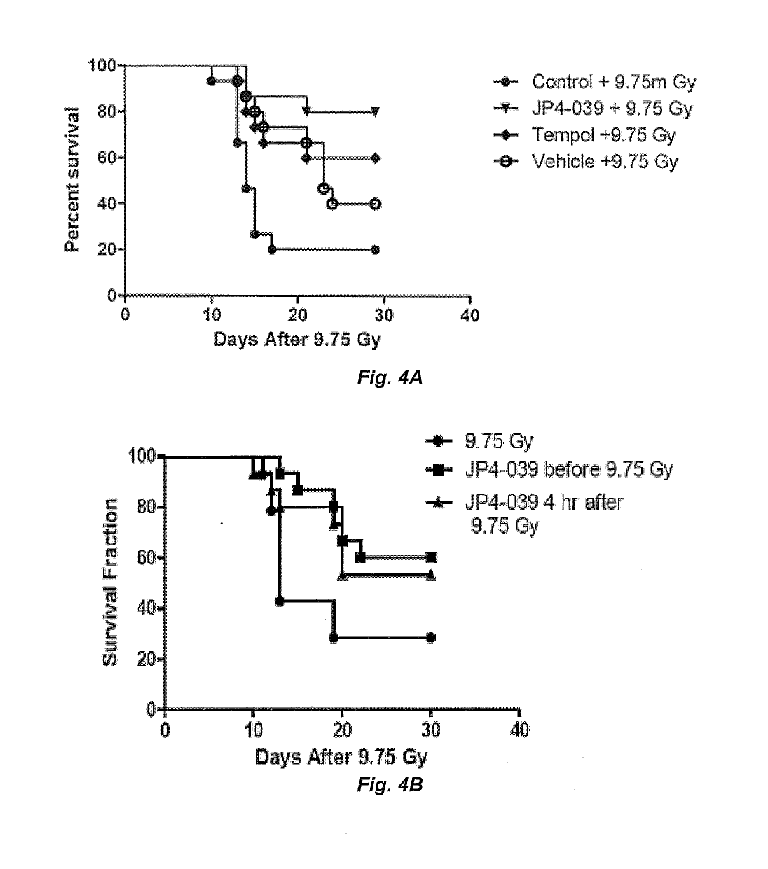

FIGS. 4A and 4B are graphs showing GS-nitroxide compound JP4-039 increases survival of mice exposed to 9.75 Gy total body irradiation. In FIG. 4A, mice received intraperitoneal injection of 10 mg per kilogram of each of the chemicals indicated, then 24 hours later received 9.75 Gy total body irradiation according to published methods. Mice were followed for survival according to IACUC regulations. There was a significant increase in survival of mice receiving JP4-039 compared to irradiated control mice. (P=0.0008). In FIG. 4B, mice received intraperitoneal injection of JP4-039 either 10 minutes before (square symbols) or 4 hours after (triangle symbols) irradiation with 9.75 Gy.

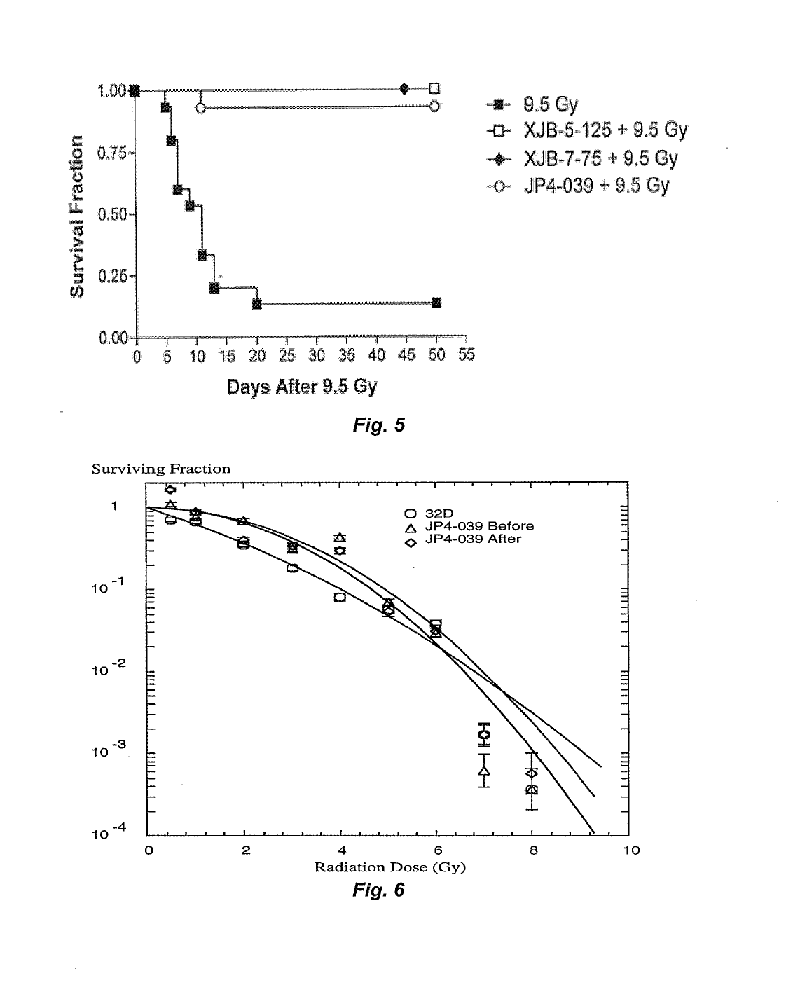

FIG. 5 is a graph showing that GS-nitroxide compound JP4-039 increases survival of mice exposed to 9.5 Gy total body irradiation. Groups of 15 mice received intraperitoneal injection of 10 mg. per kilogram of each indicated GS-nitroxide compound or carrier (Cremphora plus alcohol at 1 to 1 ratio, then diluted 1 to 10 in distilled water). Mice received 10 mg per kilogram intra-peritoneal injection 24 hours prior to total body irradiation. Control mice received radiation alone. There was a statistically significant increase in survival in mice receiving GS-nitroxide compounds. (P=0.0005)

FIG. 6 is a graph showing that GS-nitroxide JP4-039 is an effective hematopoietic cell radiation mitigator when delivered 24 hr after irradiation. Irradiation survival curves were performed on cells from the 32D cl 3 mouse hematopoietic progenitor cell line, incubated in 10 μM JP4-039 for 1 hour before irradiation, or plated in methylcellulose containing 10 μM JP4-030 after irradiation. Cells were irradiated from 0 to 8 Gy, plated in 0.8% methylcellulose containing media, and incubated for 7 days at 37° C. Colonies of greater than 50 cells were counted and data analyzed by linear quadratic and single-hit, multi-target models. Cells incubated in JP4-039 were more resistant as demonstrated by an increased shoulder on the survival curve with an ñ of 5.25±0.84 if drug was added before irradiation or 4.55±0.47 if drug was added after irradiation compared to 1.29±0.13 for 32D cl 3 cells alone (p=0.0109 or 0.0022, respectively).

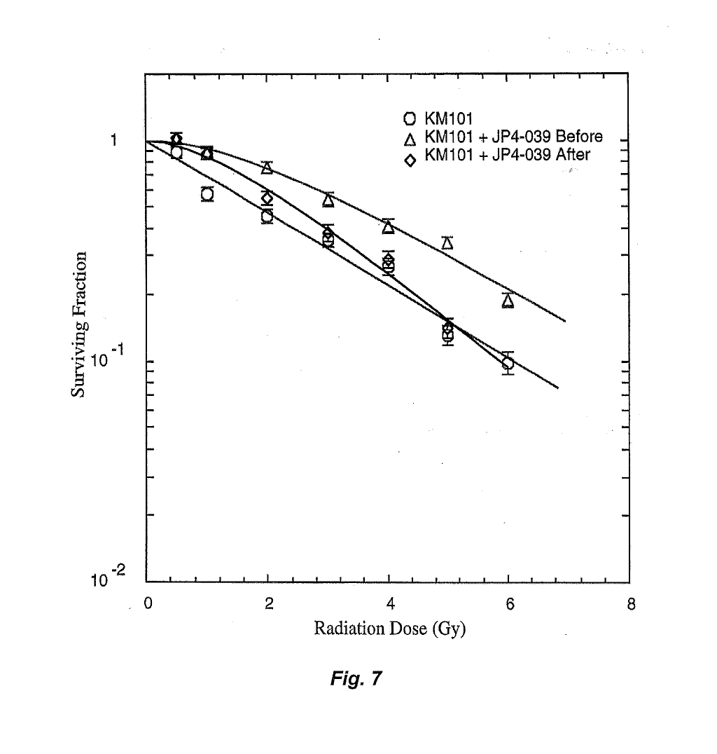

FIG. 7 is a graph showing that JP4-039 is an effective mitigator of irradiation damage to KM101 human marrow stromal cells. KM101 cells were incubated in media alone or in JP4-039 (10 μM) for one hour before irradiation or 24 hours after irradiation. The cells were irradiated to doses ranging from 0 to 6 Gy and plated in 4 well plates. Seven days later the cells were stained with crystal violet and colonies of greater than 50 cells counted. Cells incubated in JP4-039 either before or after irradiation were more radioresistant as shown by an increased shoulder of n=2.3±0.2 or 2.2±0.2, respectively compared to n of 1.1±0.1 for the KM101 cells (p=0.0309 or 0.0386, respectively). There was no significant change in the Do for the different conditions.

### Example 3

The following can be used to select and optimize the best GS-nitroxide JP4-039 (radiation damage mitigator drug) that can enhance human bone marrow stromal cell and fresh human stromal cell line seeding efficiency into irradiated limbs of NOD/SCID mice. MnSOD-overexpressing cells are a positive control.

(a) Experiments with KM101-MnSOD/Ds-Red (Control KM101-Ds-Red) Clonal Cell Lines.

Groups of 12 NOD/SCID mice receive 300 cGy total body irradiation (low dose leg) and a 1000 cGy boost to the left hind leg (high dose leg), then 24 hours later intravenous injection of 1×10⁵ or 1×10⁶ cells of each cell line (groups 1 and 2). Group 3 is mice that receive MnSOD-PL intravenously 24 hours prior to irradiation and then injection of KM101-MnSOD/ds-red. Group 4 is mice that receive MnSOD-PL intravenously 24 hours prior to irradiation, then control KM101/ds-red cells. This experiment may be repeated twice. Mice will have bone marrow flushed from the hind limbs at days 1, 3, 7, 14 after cell transplantation, and scoring of the percent of total cells and number of colony forming cells recoverable which are ds-red positive, thus of human origin. The scoring may be by ds-red positivity, and then by colony formation in vitro by stromal cells. The total, then the percent of stromal cells of human origin is then be scored.

(B) Experiments Demonstrating Improvement in Human Bone Marrow Stromal Cell Line KM101 Seeding by Mitochondrial Targeted Radiation Protection/Mitigation JP4-039 (GS-Nitroxide) Administration.

This experiment is conducted essentially as described above (A), with all groups, but with a sub-group receiving JP4-039 24 hours after radiation (same day as cell lines are injected, or a sub-group receiving intraperitoneal JP4-039 (daily or weekly after cell line transplantation). Cells are explanted from the high dose and low dose irradiated femurs at days 7, 14, 21, and cultured in vitro for human stromal colony forming progenitor cells (CFU-F). The percent and total number of human cells entering the high dose and low dose irradiated limbs is quantitated by cell sorting for ds-red, Each experiment can be completed twice.

Experiments as in (A) above, but substituting fresh human marrow Stro1+ stromal cells from a 45 y.o. donor, are performed

Experiments as in (B) above substituting Stro1+ human marrow stromal cells are performed.

Statistical Considerations—

In (A), comparisons occur at 4 different time points between 4 groups where either MnSOD-PL or no MnSOD-PL, and either 10⁵ or 10⁶ KM101 cells are injected, in terms of the number of ds-red-KM101 cells. In (B), comparisons occur at 3 different time points between 10 groups where different doses and schedules of the experimental compound will be used, in terms of the same endpoint as in (A). (C) and (D) are the same as (A) and (B) respectively, except that human stromal cells are used in place of KM101 cells. All the comparisons in this task are performed separately for high and low dose radiated legs. ANOVA followed by Tukey's test can be used for these analyses. Sample size can be estimated by the two sample t-test for pairwise comparisons. Sample size estimation is based on the expected difference to detect between groups in terms of the common standard deviation a. Six mice per group can be sacrificed per time point. With this sample size, there will be 82% power to detect a difference of 1.8σ between groups using the two sided two sample t test with significance level 0.05.

As the secondary endpoint, the number of colony forming unit fibroblast (human) CFU-F can also be compared between groups with the same method as the primary endpoint.

It is expected that MnSOD overexpression in KM101-MnSOD/ds-red cells will lead to a higher seeding efficiency into both the high and low dose irradiated limbs of NOD/SCID mice. It is expected that MnSOD-PL treatment of the hematopoietic microenvironment prior to KM101 clonal line cell line infusion will further enhance engraftment of both KM101-MnSOD/ds-red and KM101-ds-red cell lines. It is expected that the highest percent of seeding efficiency will be detected in the mice receiving MnSOD-PL prior to irradiation and injection of KM101-MnSOD/ds-red cells.

It is expected that JP4-039 administration daily after cell transplantation will facilitate improved stability of engraftment of all stromal cell lines by decreasing free radical production by the irradiated marrow microenvironment.

An inactive control compound for JP4-039 may be used, (JP4-039 absent the nitroxide active moiety or the specific formulation used as a vehicle). Based upon the results of these experiments, the optimal condition for bone marrow stromal cell seeding is derived, and these conditions are used in experiments described below.

### Example 4

Selection and optimization of a GS-nitroxide JP4-039 therapy to enhance human CD34+ cord blood multilineage hematopoietic stein cell progenitor cell seeding into irradiated limbs of NOD/SCID mice that have been prepared by engraftment of human marrow stromal cells.
i. Experiments are conducted with TBI treated C57BL/6J mice and mouse marrow screening. (preliminary system test).
ii. Experiments using the optimal seeding protocol for human KM101 cells into irradiated NOD/SCID mice (anticipated to be those mice receiving MnSOD-PL prior to irradiation, and then injection with KM101-MnSOD/ds-red, supplemented with JP4-039 daily, each group contains 12 mice) are conducted. Mice then receive intravenous injection of 1×10⁵ or 1×10⁶ CD34+ LIN− cells from human umbilical cord blood origin. Control cells may be CD34+ LIN+(differentiated progenitor) cells 10⁵ or 10⁶ per injection.

These experiments may be carried out in two schedules:
i. Injection of cord blood cells at the same time as KM101-MnSOD/ds-red cells.
ii of cord blood cells at time of optimal recovery of KM101-MnSOD/ds-red cells from the explant experiments of Example 3. This should be at day 7 or day 14 after stromal cell injection
   In these experiments, mice are followed and tested at serial time points out to two months after cord blood stem cell transplantation. The percent of human peripheral blood hematopoietic cells is scored in weekly peripheral blood samples and number of cells forming CFU-GEMM colonies is tested in explanted bones from sacrificed mice.

At days 7, 14, 21, 28, or 60 after cord blood transplantation, mice in sub-groups are sacrificed, and all cells flushed from the high dose and low dose irradiated femurs, and assays carried out for human multilineage hematopoietic progenitors-CFU-GEMM. Assays may be carried out by two methods:
i. Sorting human CD34+ cells with monoclonal antibodies specific for human.
ii. Colony formation in human CFU-GEMM culture medium and then secondary scoring of human colonies as the subset of total mouse and human colony forming cells detected at days 7 and days 14 in vitro.

In vitro experiments may be carried out in parallel as follows:

KM101-MnSOD-PL plateau phase stromal cells are irradiated in vitro to 100, 200, 500, 1000 cGy, and then CD34+ LIN− human cord blood cells co-cultivated with the stromal cells in vitro. Controls include unirradiated KM101-MnSOD/ds-red, irradiated KM101-ds-red cells, unirradiated KM101-ds-red.

Scoring is done on human cobblestone islands (stem cell colonies) on these cultures on a weekly basis, plots of cumulative cobblestone island formation are formed, cumulative non-adherent cell production with weekly cell harvest are assessed, and assay of weekly cell harvest for CFU-GEMM formation is also utilized. These studies may be carried out over two-three weeks. In vitro co-cultivation studies can only partially duplicate the in vivo hematopoietic microenvironment, and thus two weeks should be the maximum efficient time for detection of whether MnSOD-PL expression in the adherent KM101 layer will increase engraftment of cord blood stem cells.

Experiments with JP4-039 supplementation of the cord blood transplantation program as above are carried out to increase homing, stable quiescence, and repopulation capacity of human cord blood stem cells by removing ROS production in the irradiated marrow stromal cell environment.

Experiments In Vitro Supplementing in Co-Cultivation Culture Media the Drug JP4-039 Daily.

The experiments with irradiated KM101 subclonal lines, co-cultivated with cord blood stem cells are carried out with the addition of JP4-039, or an active analog of JP4-039, daily. Control experiments include addition of CD34+ LIN+ differentiated cord blood cells that are expected to produce fewer CFU-GEMM over time. Stromal cell cultures are irradiated, cord blood cells added, and cultures scored as above.

Groups of 12 mice receive the optimal protocol for human CFU-GEMM cell engraftment from the experiment above, and then sub-groups are treated as follows:
i. JP4-039 twice weekly.
ii. JP4-039 daily.
iii. Inactive JP4-039 analog daily.

Experiments as above, substituting fresh human Stro1+ marrow cells for KM101 subclonal lines, are performed.

Experiments as above, substituting human Stro1+ marrow cells for KM101 subclonal lines, are performed.

### Statistical Considerations—

Comparisons are made at 5 different time points between 7 groups where MnSOD-KM101 and/or 10⁵/10⁶ CD34+ cells are used, in terms of the number of CD45+ cells. Comparisons at 5 different time points between 7 groups that use KM101, CD34+ cells, KM101 plus CD34+ cells, the experimental compound single or double administrations, or inactive analog of the experimental compound single or double administrations, in terms of the same endpoint as above are also performed. Tasks involving cell culture are the same as (A) and (B) of Example 3, respectively, except that human Stro1+ marrow cells are used in place of KM101 cells. All the comparisons in this task can be performed separately for high and low dose radiated legs. ANOVA followed by Tukey's test can be used for these analyses. Similar to the sample size considerations in Example 3, one may use 6 mice per group at each time point. As the secondary endpoint, the number of CFU-GEMM can also be compared between groups with the same method as the primary endpoint.

Likely Outcomes—Based on the results of Example 3, it is expected that cord blood stem cell and human bone marrow stromal cell homing in vitro will be optimized by MnSOD-PL treatment of the mouse microenvironment prior to stromal cell transplantation, and that MnSOD-PL overexpressing KM101 cells will show further stability in the irradiated microenvironment. It is expected that JP4-039 treatment will further enhance hematopoietic cell survival and increase CFU-GEMM in numbers.

### Example 5

### Alternative Designs of Nitroxide Analogues

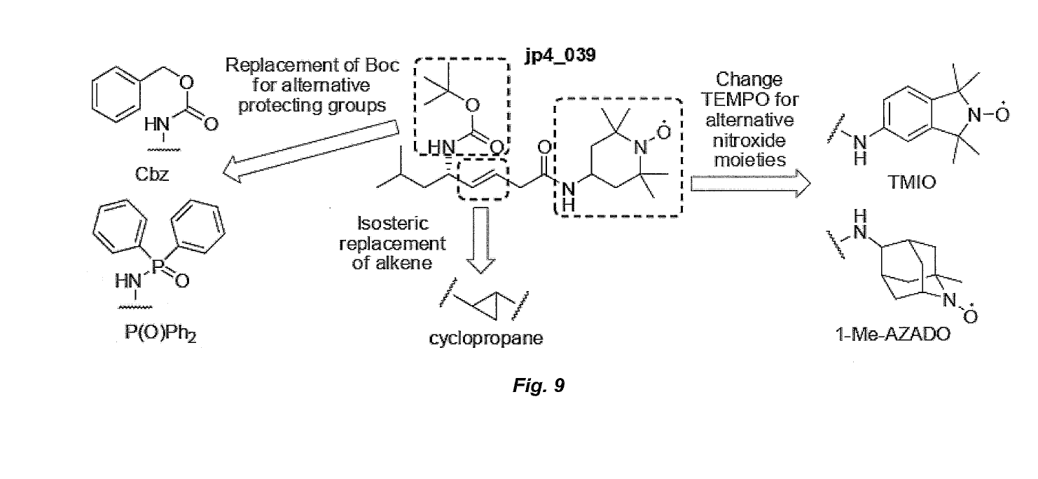

To further investigate the structural requirements for high activity of GS-nitroxide compound JP4-039, we have designed several nitroxide analogues. FIG. 9 shows a schematic of alternative designs of nitroxide analogues. The design can encompass one or both of: modification of the targeting group to optimize the drug-like properties and/or investigation of alternative nitroxide containing groups to improve their oxidant efficiency (for example and without limitation, see Reid, D. A. et al. The synthesis of water soluble isoindoline nitroxides and a pronitroxide hydroxylamine hydrochloride UV-VIS probe for free radicals. Chem Comm 1998, 17:1907-8; Iwabuchi, Y. J., Exploration and Exploitation of Synthetic Use of Oxoammonium Ions in Alcohol Oxidation. J. Synth. Org. Chem. Jpn. 2008, 66(11):1076-84). Modification of the targeting group can include replacement of Boc for alternative protecting groups, such as Ac (—C(O)CH₃), Cbz (—C(O)O-Bn, where Bn is a benzyl group) or dialkylphosphates. Dialkylphosphates include —P(O)-Ph₂, where Ph is a phenyl group. Other modifications also include isosteric replacement of the alkene group within the targeting group, such as with a cyclopropane group. The nitroxide containing group includes TEMPO and TEMPOL, as well as alternative nitroxide moieties, such as TMIO (1,1,3,3-tetramethylisoindolin-2-yloxyl) or 1-Me-AZADO (1-methyl 2-azaadamantane N-oxyl). Synthesis protocols of these alternative nitroxide moieties are provided below.

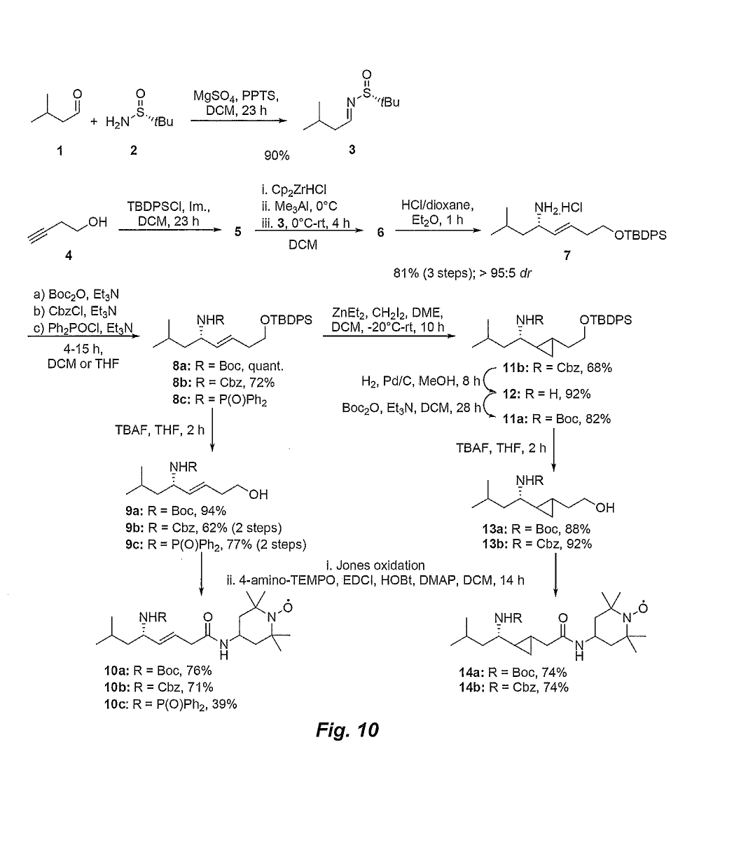

FIG. 10 shows a synthetic protocol that can be used to produce various alternative designs of nitroxide analogues, including JP4-039, compounds according to Formula 2, compounds according to Formula 3, and other analogues. The specific synthesis of JP4-039 has been described above in Example 1. JP4-039 and its analogues were prepared via an efficient method for the asymmetric synthesis of allylic amines, previously developed in our laboratory (Wipf P. & Pierce J. G. Expedient Synthesis of the α-C-Glycoside Analogue of the Immunostimulant Galactosylceramide (KRN7000), Org. Lett. 2006, 8(15):3375-8). One key step in FIG. 10 includes use of the zirconium methodology to produce a diastereomeric allylic amine (7). This methodology includes hydrozirconation of alkyne (5) with Cp₂ZrHCl, transmetalation to Me₃Al, and addition to N-tBu-sulfinyl amine (3). The Smith cyclopropanation of the alkene (8b) with Zn(CH₂I)₂ is another key step in FIG. 10. In this latter step, the stereochemistry around the cyclopropane ring is to be determined after the reaction.

Synthesis of compounds (10a, JP4-039), (10b), (10c), (14a), and (14b) (shown in FIG. 10) was accomplished according to the following.

### (R,E)-2-Methyl-N-(3-methylbutylidene)propane-2-sulfinamide (3)

The synthesis of the title compound has already been described in Example 1 (compound 1).

### (But-3-ynyloxy)(tert-butyl)diphenylsilane (5)

The synthesis of the title compound has already been described in Example 1 (compound 2).

### (S,E)-8-(tert-Butyldiphenylsilyloxy)-2-methyloct-5-en-4-amine hydrochloride (7)

The synthesis of the title compound has already been described in Example 1 (compound 3).

### (S,E)-tert-Butyl 8-(tert-butyldiphenylsilyloxy)-2-methyloct-5-en-4-ylcarbamate (8a)

The synthesis of the title compound has already been described in Example 1 (compound 4).

### (S,E)-Benzyl 8-(tert-butyldiphenylsilyloxy)-2-methyloct-5-en-4-ylcarbamate (8b)

To a mixture of the amine 7 (1.50 g, 3.79 mmol) in dry THF (15 mL) were added Et₃N (1.65 mL, 11.75 mmol), and then a solution of benzyl chloroformate (CbzCl, 0.59 mL, 4.17 mmol) in dry THF (4 mL) at 0° C. The resulting white suspension was allowed to warm to rt and stirred for 5 h, then diluted with DCM and water. The aqueous phase was extracted with DCM (2×), and the combined organic layers were washed with 10% HCl and sat. NaHCO₃, dried (MgSO₄), filtered and concentrated in vacuo. Flash chromatography (SiO₂, 8:2, hexanes/EtOAc) afforded 1.45 g (72%) of the title compound as a yellow oil. ¹H NMR (300 MHz, CDCl₃) δ 7.75-7.65 (m, 4H), 7.50-7.28 (m, 11H), 5.70-5.55 (m, 1H), 5.40 (dd, 1H, J=15.4, 6.2 Hz), 5.11 (s, 2H), 4.58 (m, 1H), 4.21 (m, 1H), 3.71 (t, 2H, J=6.6 Hz), 2.30 (q, 2H, J=6.6 Hz), 1.67 (m, 1H), 1.40-1.22 (m, 2H), 1.07 (s, 9H), 0.92 (in, 6H); HRMS (ESI) in/z calcd for C₃₃H₄₃NO₃SiNa 552.2910. found 552.2930.

### (S,E)-N-(8-(tert-Butyldiphenylsilyloxy)-2-methyloct-5-en-4-yl)-P,P-diphenylphosphinic amide (8c)

To a solution of the amine 7 (400 mg, 1.01 mmol) in dry DCM (7 mL) were added Et₃N (0.44 mL, 3.13 mmol), and then a solution of diphenylphosphinic chloride (Ph₂POCl, 0.22 mL, 1.11 mmol) in dry DCM (3 mL) at 0° C. After being stirred at 0° C. for 15 min, the reaction mixture was allowed to warm to rt and stirred for 4 h, then diluted with DCM and 10% HCl. The aqueous phase was extracted with DCM and the combined organic layers were washed with sat. NaHCO₃, dried (MgSO₄), filtered and concentrated in vacuo to afford 720 mg of the crude title compound as a pale yellow solidified oil, which was used for the next step without further purification.

### (S,E)-tert-Butyl 8-hydroxy-2-methyloct-5-en-4-ylcarbamate (9a)

The synthesis of the title compound has already been described in Example 1 (compound 5).

(S,E)-Benzyl 8-hydroxy-2-methyloct-5-en-4-ylcarbamate (9b). To a solution of the TBDPS-protected alcohol 8b (584 mg, 1.10 mmol, crude) in dry THF (9 mL) at 0° C. was added TBAF (1.0M/THF, 1.38 mL, 1.38 mmol), and the reaction mixture was allowed to warm to rt while stirring under argon for 3.5 h, then quenched with sat. aq. NH₄Cl and diluted with EtOAc. The aqueous phase was separated and extracted with EtOAc. The combined organic layers were washed with brine, dried (Na₂SO₄), filtered and concentrated in vacuo. Flash chromatography (SiO₂, 5:5, hexanes/EtOAc) afforded 194 mg (60%, 2 steps) of the title compound as a colorless oil. [α]_{D}²³ −6.4 (c 1.0, DCM); ¹H NMR (300 MHz, CDCl₃) δ 7.20-7.40 (m, 5H), 5.65-5.49 (m, 1H), 5.44 (dd, 1H, J=15.3, 6.6 Hz), 5.09 (s, 2H), 4.67 (bs, 1H), 4.16 (m, 1H), 3.63 (bs, 2H), 2.28 (q, 2H, J=6.0 Hz), 1.82 (bs, 1H), 1.65 (m, 1H), 1.40-1.25 (m, 2H), 0.80-1.00 (m, 6H); HRMS (ESI) m/z calcd for C₁₇H₂₅NO₃Na 314.1732. found 314.1739.

### (S,E)-N-(8-Hydroxy-2-methyloct-5-en-4-yl)-P,P-diphenylphosphinic amide (9c)

To a solution of the TBDPS-protected alcohol 8c (700 mg, 0.983 mmol, crude) in dry THF (8 mL) at 0° C. was added TBAF (1.0M/THF, 1.23 mL, 1.23 mmol), and the reaction mixture was allowed to warm to rt while stirring under argon. As completion was not reached after 4 h, 0.75 eq of TBAF (0.75 mL) was added at 0° C. The reaction mixture was stirred further at rt for 3 h, then quenched with sat. aq. NH₄Cl and diluted with EtOAc. The aqueous phase was separated and extracted with EtOAc. The combined organic layers were washed with brine, dried (Na₂SO₄), filtered and concentrated in vacuo. Flash chromatography (SiO₂, 95:5, EtOAc/MeOH) afforded 272 mg (77%, 2 steps) of the title compound as a white solid. mp 124.0-124.2° C.; [α]_{D}²³-12.1 (c 1.0, DCM); ¹H NMR (300 MHz, CDCl₃) δ 8.00-7.83 (m, 4H), 7.58-7.35 (m, 6H), 5.52 (dd, 1H, J=15.3, 9.0 Hz), 5.24 (m, 1H), 4.58 (bs, 1H), 3.78-3.47 (m, 3H), 2.80 (appdd, 1H, J=9.2, 3.8 Hz), 2.16 (m, 2H), 1.68 (bs, 1H), 1.55-1.43 (m, 1H), 1.43-1.31 (m, 1H), 0.87 (dd, 6H, J=8.6, 6.4 Hz); HRMS (ESI) m/z calcd for C₂₁H₂₈NO₂PNa 380.1755. found 380.1725.

### TEMPO-4-yl-(S,E)-5-(tert-butoxycarbonylamino)-7-methyloct-3-enamide (10a, JP4-039)

The synthesis of the title compound has already been described in Example 1 (compound 7).

### TEMPO-4-yl-(S,E)-5-(benzyloxycarbonylamino)-7-methyloct-3-enamide (10b)

To a solution of the alcohol 9b (158 mg, 0.543 mmol) in acetone (5 mL) at 0° C. was added slowly a freshly prepared solution of Jones reagent (2.5M, 0.54 mL, 1.358 mmol). The resulting dark suspension was stirred at 0° C. for 1 h, then diluted with Et₂O and water. The aqueous phase was separated and extracted with Et₂O (2×). The combined organic layers were washed with water (2×) and brine (1×), dried (Na₂SO₄), filtered and concentrated in vacuo to yield 166 mg (quant.) of the crude acid as a slightly yellow oil, that was used for the next step without further purification.

To a solution of this acid (160 mg, 0.524 mmol, crude) in dry DCM (7 mL) at 0° C. were added successively a solution of 4-amino-TEMPO (139 mg, 0.786 mmol) in dry DCM (0.5 mL), DMAP (71 mg, 0.576 mmol), HOBt.H₂O (78 mg, 0.576 mmol) and EDCI (123 mg, 0.629 mmol). The resulting orange solution was stirred at room temperature under argon for 15 h, and then washed with sat. NH₄Cl. The aqueous phase was separated and extracted once with DCM, and the combined organic layers were dried (Na₂SO₄), filtered and concentrated in vacuo. Flash chromatography (SiO₂, 5:5 to 3:7, hexanes/EtOAc) afforded 171 mg (71%) of the title compound as a peach colored foam. mp 60.5° C. (softening point: 44° C.); [α]_{D}²³+26.5 (c 0.5, DCM); EIMS m/z 458 ([M]⁺, 37), 281 (19), 154 (28), 124 (47), 91 (100), 84 (41); HRMS (EI) m/z calcd for C₂₆H₄₀N₃O₄ 458.3019. found 458.3035.

### TEMPO-4-yl-(S,E)-5-(diphenylphosphorylamino)-7-methyloct-3-enamide (10c)

To a solution of the alcohol 9c (166.5 mg, 0.466 mmol) in acetone (5 mL) at 0° C. was slowly added a freshly prepared solution of Jones reagent (2.5M, 0.47 mL, 1.165 mmol). The resulting dark suspension was stirred at 0° C. for 2 h, then diluted with Et₂O and water. The aqueous phase was separated and extracted with Et₂O (2×). The combined organic layers were washed with water (2×) and brine (1×), dried (Na₂SO₄), filtered and concentrated in vacuo to yield 114 mg (66%) of the crude acid as a white foam, that was used for the next step without further purification.

To a solution of this acid (110 mg, 0.296 mmol, crude) in dry DCM (3.5 mL) at 0° C. were added successively a solution of 4-amino-TEMPO (78.4 mg, 0.444 mmol) in dry DCM (0.5 mL), DMAP (40.2 mg, 0.326 mmol), HOBt.H₂O (44.0 mg, 0.326 mmol) and EDCI (69.5 mg, 0.355 mmol). The resulting orange solution was stirred at room temperature under argon for 13 h, and then washed with sat. NH₄Cl. The aqueous phase was separated and extracted once with DCM, and the combined organic layers were dried (Na₂SO₄), filtered and concentrated in vacuo. Flash chromatography (SiO₂, EtOAc to 97:3, EtOAc/MeOH) afforded 91.2 mg (59%) of the title compound as an orange oil which solidified very slowly upon high vacuum. mp 168.0-168.8° C. (softening point: ˜75° C.); [α]_{D}²³ −14.1 (c 0.5, DCM); EIMS m/z 525 ([M+H]⁺, 10), 371 (27), 218 (28), 201 (74), 124 (100), 91 (35), 84 (26); HRMS (EI) m/z calcd for C₃₀H₄₃N₃O₃P 524.3042. found 524.3040.

### Benzyl(1S)-1-(2-(2-(tert-butyldiphenylsilyloxy)ethyl)cyclopropyl)-3-methylbutylcarbamate (11b)

To a solution of ZnEt₂ (110 mg, 0.844 mmol) in dry DCM (2 mL) was added DME (distilled, 0.088 mL, 844 mmol). The reaction mixture was stirred at room temperature for 10 min under N₂, then cooled to −20° C. and CH₂I₂ (0.137 mL, 1.687 mmol) was added dropwise over 4 min. After stirring for 10 min, a solution of the alkene 8b (149 mg, 0.281 mmol) in dry DCM (1 mL) was added dropwise over 5 min. The reaction mixture was allowed to warm to room temperature while stirring. After 10 h, the reaction mixture was quenched with sat. aq. NH₄Cl and diluted with DCM and water, the aqueous phase was separated and extracted with EtOAc. The combined organic layers were dried (Na₂SO₄), filtered and concentrated in vacuo. Flash chromatography (SiO₂, 9:1, hexanes/Et₂O) afforded 785 mg (68%) of the title compound as a colorless oil. ¹H NMR analysis showed only 1 diastereomer (>95:5 dr). [α]_{D}²³ −26.8 (c 1.0, DCM); ¹H NMR (300 MHz, CDCl₃) δ 7.73-7.66 (m, 4H), 7.48-7.28 (m, 11H), 5.13-4.96 (m, 2H), 4.62 (appbd, 1H, J=8.4 Hz), 3.72 (appbt, 2H, J=6.4 Hz), 3.21 (in, 1H), 1.80-1.63 (m, 1H), 1.60-1.25 (m, 4H), 1.08 (s, 9H), 0.92 (appd, 6H, J=6.3 Hz), 0.79 (in, 1H), 0.51 (m, 1H), 0.40 (m, 1H), 0.30 (m, 1H); HRMS (ESI) m/z calcd for C₃₄H₄₅NO₃SiNa 566.3066. found 566.3103.

### (1S)-1-(2-(2-(Tert-butyldiphenylsilyloxy)ethyl)cyclopropyl)-3-methylbutan-1-amine (12)

A flask containing a solution of the Cbz-protected amine 11b (460 mg, 0.846 mmol) in a 5:1 MeOH/EtOAc mixture (12 mL) was purged and filled 3 times with argon, then 10% Pd/C (50 mg) was added. The flask was purged and filled 3 times with H₂, and the resulting black suspension was stirred at room temperature under H₂ (1 atm). Since the reaction did not reach completion after 3 h, an additional amount of 10% Pd/C (30 mg) was added and stirring under H₂ was continued for 5 h. The reaction mixture was then filtered through a pad of Celite, the Celite washed with MeOH and AcOEt, and the solution concentrated in vacuo to yield 317 mg (92%) of the crude title compound as a pale yellow oil, that was used for the next step without further purification.

### Tert-butyl(1S)-1-(2-(2-(tert-butyldiphenylsilyloxy)ethyl)cyclopropyl)-3-methylbutylcarbamate (11a)

To a solution of the amine 12 (309 mg, 0.755 mmol) in dry DCM (12 mL) was added Et₃N (0.21 mL, 0.153 mmol) and then Boc₂O (183 mg, 0.830 mmol) at 0° C. The reaction mixture was stirred at room temperature under N₂ for 28 h. The reaction was quenched with sat. aq. NH₄Cl and the aqueous phase extracted with DCM. The combined organic layers were dried (Na₂SO₄), filtered and concentrated in vacuo to yield 471 mg of the crude title compound as a colorless oil, that was used for the next step without further purification.

### Tert-butyl(1S)-1-(2-(2-hydroxyethyl)cyclopropyl)-3-methylbutylcarbamate (13a)

To a solution of the crude TBDPS-protected alcohol 11a (464 mg, 0.742 mmol) in dry THF (6 mL) at 0° C. was added TBAF (1.0M/THF, 0.93 mL, 0.927 mmol), and the reaction mixture was allowed to warm to room temperature while stirring under N₂. Since TLC showed incomplete reaction after 5 h, 0.75 eq. TBAF (0.56 mL) was added. After 9 h, the reaction mixture was quenched with sat. aq. NH₄Cl and diluted with EtOAc. The aqueous phase was separated and extracted with EtOAc. The combined organic layers were washed with brine, dried (Na₂SO₄), filtered and concentrated in vacuo. Flash chromatography (SiO₂, 5:5, hexanes/EtOAc) afforded 177 mg (88%) of the title compound as a colorless oil which solidified upon high vacuum to give a white powder. mp 49.8-50.2° C. [α]_{D}22 −30.8 (c 1.0, DCM); ¹H NMR (300 MHz, CDCl₃) δ 4.50 (appbd, 1H, J=4.5 Hz), 3.66 (bs, 2H), 2.94 (m, 1H), 2.36 (bs, 1H), 1.82 (bs, 1H), 1.71 (m, 1H), 1.45 (s, 9H), 1.39 (t, 2H, J=7.2 Hz), 1.01 (bs, 2H), 0.90 (dd, 6H, J=10.2, 6.6 Hz), 0.50 (m, 1H), 0.43-0.27 (in, 2H); HRMS (ESI) in/z calcd for C₁₅H₂₉NO₃Na 294.2045. found 294.2064.

### Benzyl(1S)-1-(2-(2-hydroxyethyl)cyclopropyl)-3-methylbutylcarbamate (13b)

To a solution of the TBDPS-protected alcohol 11b (320 mg, 0.588 mmol) in dry THF (5 mL) at 0° C. was added TBAF (1.0M/THF, 0.74 mL, 0.735 mmol), and the reaction mixture was allowed to warm to rt while stirring under argon for 7 h, then quenched with sat. aq. NH₄Cl and diluted with EtOAc. The aqueous phase was separated and extracted with EtOAc. The combined organic layers were washed with brine, dried (Na₂SO₄), filtered and concentrated in vacuo. Flash chromatography (SiO₂, 5:5, hexanes/EtOAc) afforded 166 mg (92%) of the title compound as a colorless oil. [α]_{D}²³ −21.6 (c 1.0, DCM); ¹H NMR (300 MHz, CDCl₃) δ 7.42-7.28 (m, 5H), 5.10 (m, 2H), 4.76 (appbd, 1H, J=5.7 Hz), 3.63 (bs, 2H), 3.04 (in, 1H), 2.12-1.98 (bs, 1H), 1.83-1.62 (m, 2H), 1.42 (t, 2H, J=7.0 Hz), 1.16-0.95 (m, 2H), 0.90 (appt, 6H, J=7.0 Hz), 0.53 (sept, 1H, J=4.3 Hz), 0.42 (dt, 1H, J=8.4, 4.5 Hz), 0.34 (dt, 1H, J=8.4, 5.0 Hz); HRMS (ESI) m/z calcd for C₁₈H₂₇NO₃Na 328.1889. found 328.1860.

### TEMPO-4-yl-2-(2-((S)-1-(tert-butoxycarbonylamino)-3-methylbutyl)cyclopropyl)acetamide (14a)

To a solution of the alcohol 13a (130 mg, 0.477 mmol) in acetone (5 mL) at 0° C. was slowly added a solution of Jones reagent (2.5M, 0.48 mL, 1.194 mmol). The resulting dark suspension was stirred at 0° C. for 1 h, then diluted with Et₂O and water. The aqueous phase was separated and extracted with Et₂O (2×). The combined organic layers were washed with water (2×) and brine (1×), dried (Na₂SO₄), filtered and concentrated in vacuo to yield 133 mg (97%) of the crude title compound as a colorless oil, that was used for the next step without further purification.

To a solution of this acid (127.6 mg, 0.447 mmol, crude) in dry DCM (5.5 mL) at 0° C. were added successively a solution of 4-amino-TEMPO (118.4 mg, 0.671 mmol) in dry DCM (0.5 mL), DMAP (60.7 mg, 0.492 mmol), HOBt.H₂O (66.4 mg, 0.492 mmol) and EDCI (105.0 mg, 0.536 mmol). The resulting orange solution was stirred at it under argon for 15 h, and then washed with sat. NH₄Cl. The aqueous phase was separated and extracted once with DCM, and the combined organic layers were dried (Na₂SO₄), filtered and concentrated in vacuo. Flash chromatography (SiO₂, 5:5 to 3:7, hexanes/EtOAc) afforded 150.0 mg (76%) of the title compound as a peach colored foam. mp 139.5° C.; [α]_{D}²³ −15.7 (c 0.5, DCM); EIMS m/z 438 ([M]⁺, 6), 252 (57), 140 (67), 124 (80), 91 (48), 84 (59), 57 (100); HRMS (EI) m/z calcd for C₂₄H₄₄N₃O₄ 438.3332. found 438.3352.

### TEMPO-4-yl-2-(2-4S)-1-(benzyloxycarbonylamino)-3-methylbutyl)cyclopropyl)acetamide (14b)

To a solution of the alcohol 13b (110.5 mg, 0.362 mmol) in acetone (5 mL) at 0° C. was slowly added a solution of Jones reagent (2.5M, 0.36 mL, 0.904 mmol). The resulting dark suspension was stirred at 0° C. for 1 h, then diluted with Et₂O and water. The aqueous phase was separated and extracted with Et₂O (2×). The combined organic layers were washed with water (2×) and brine (1×), dried (Na₂SO₄), filtered and concentrated in vacuo to yield 113.5 mg (98%) of the crude title compound as a colorless oil, that was used for the next step without further purification.

To a solution of this acid (110 mg, 0.344 mmol, crude) in dry DCM (4.5 mL) at 0° C. were added successively a solution of 4-amino-TEMPO (91.2 mg, 0.517 mmol) in dry DCM (0.5 mL), DMAP (46.7 mg, 0.379 mmol), HOBt.H₂O (51.2 mg, 0.379 mmol) and EDCI (80.8 mg, 0.413 mmol). The resulting orange solution was stirred at it under argon for 18 h, and then washed with sat. NH₄Cl. The aqueous phase was separated and extracted once with DCM, and the combined organic layers were dried (Na₂SO₄), filtered and concentrated in vacuo. Flash chromatography (SiO₂, 4:6, hexanes/EtOAc) afforded 123 mg (75%) of the title compound as a peach colored foam. mp 51.8° C. (softening point: 44° C.); [α]_{D}²³ −15.3 (c 0.5, DCM); EIMS m/z 472 ([M]⁺, 42), 415 (58), 322 (43), 168 (47), 140 (46), 124 (75), 91 (100), 84 (53); HRMS (EI) m/z calcd for C₂₇H₄₂N₃O₄ 472.3175. found 472.3165.

### Example 6

### Synthesis of Alternative Nitroxide Moieties

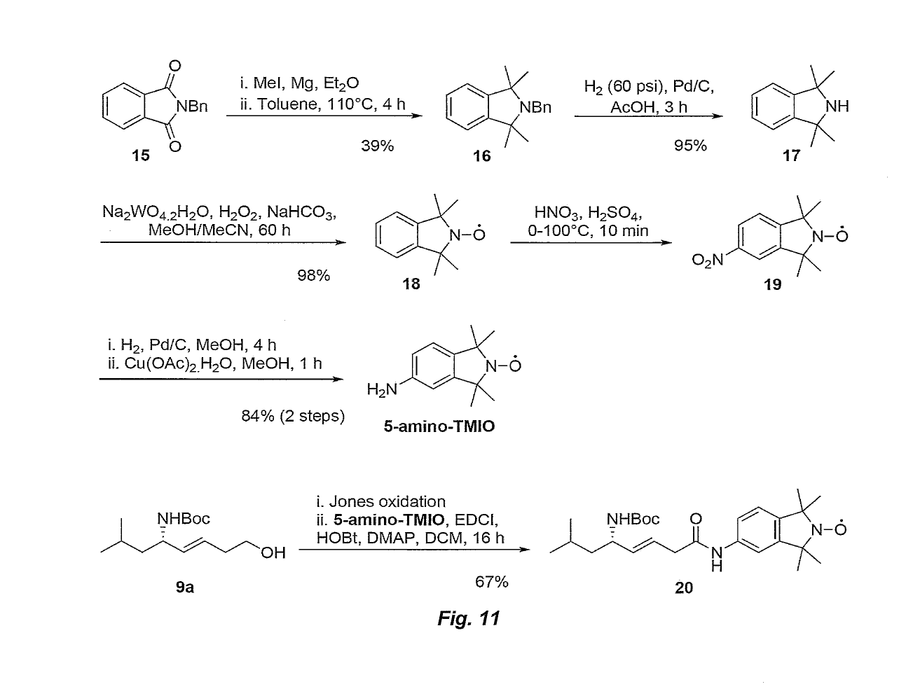

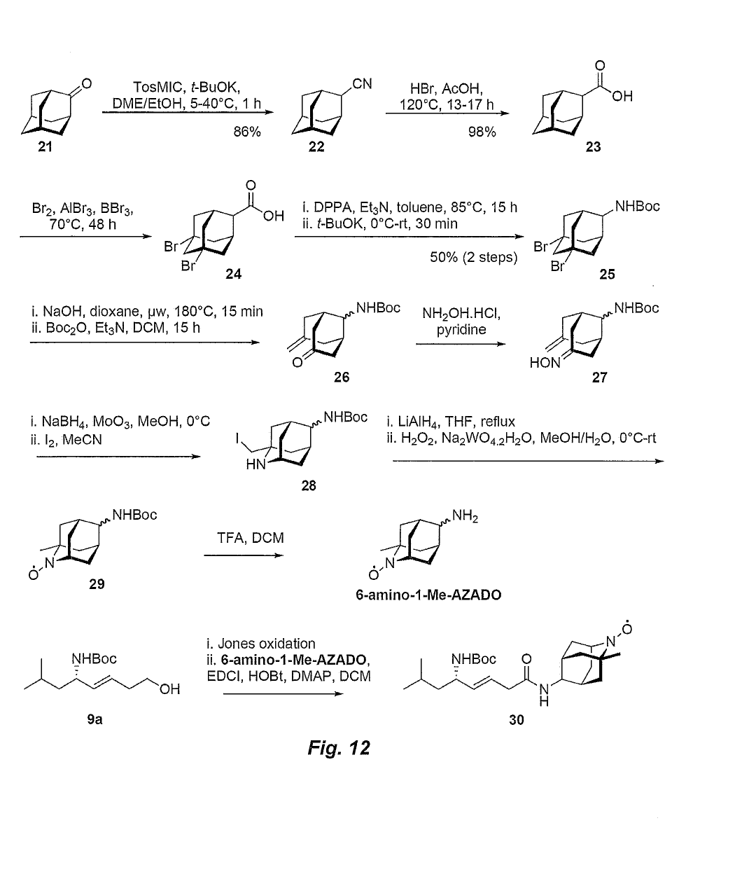

Schematics are shown for alternative nitroxide moieties, where FIG. 11 shows a synthesis protocol for 5-amino-1,1,3,3-tetramethylisoindolin-2-yloxyl (5-amino-TMIO) and FIG. 12 shows a synthesis protocol for 6-amino-1-methyl 2-azaadamantane N-oxyl (6-amino-1-Me-AZADO).

Compounds 5-amino-1,1,3,3-tetramethylisoindolin-2-yloxyl (5-amino-TMIO) and (20) are shown in FIG. 11 and were prepared according to the following. Synthesis of 5-amino-TMIO was previously described by Reid, D. A. et al. (The synthesis of water soluble isoindoline nitroxides and a pronitroxide hydroxylamine hydrochloride UV-VIS probe for free radicals. Chem Comm. 1998, 17, 1907-8) and references cited therein.

### 2-Benzyl-1,1,3,3-tetramethylisoindoline (16)

An oven-dried 250 mL, three-necked, round-bottom flask was flushed with nitrogen, and magnesium turnings (3.84 g, 156.5 mmol) were introduced, that were covered with dry Et₂O (9 mL). A solution of MeI (9.45 mL, 150.2 mmol) in dry Et₂O (80 mL) was then added dropwise via a dropping funnel while stirring over a period of 50 min. The resulting reaction mixture was then stirred for an additional 30 min, and then concentrated by slow distillation of solvent until the internal temperature reached 80° C. The residue was allowed to cool to 60° C., and a solution of N-benzylphthalimide (6.00 g, 25.04 mmol) in dry toluene (76 mL) was added dropwise via a dropping funnel with stirring at a sufficient rate to maintain this temperature. When the addition was complete, solvent was distilled slowly from the mixture until the temperature reached 108-110° C. The reaction mixture was refluxed at 110° C. for 4 h, then concentrated again by further solvent distillation. It was then cooled and diluted with hexanes (turned purple). The resulting slurry was filtered through Celite and washed with hexanes. The combined yellow filtrate turned dark red-purple after standing in air overnight. It was then concentrated in vacuo. The resulting purple residue was passed through a short column of basic alumina (grade I, 70-230 mesh), eluting with hexanes (˜1 L), to afford 2.585 g (39%) of the title compound as a colorless oil which solidified to give a white solid. mp 61.0-61.4° C. ¹H NMR (300 MHz, CDCl₃) δ 7.48 (appd, 2H, J=7.2 Hz), 7.34-7.19 (m, 5H), 7.18-7.11 (m, 2H), 4.00 (s, 2H), 1.31 (s, 12H); HRMS (EI) m/z calcd for C₁₉H₂₃N 265.1830. found 265.1824.

### 1,1,3,3-Tetramethylisoindoline (17)

The protected benzyl-amine 16 (1.864 g, 7.02 mmol) was dissolved in AcOH (34 mL) in a Parr flask, and 10% Pd/C (169.5 mg) was added. (The reaction was splited in 3 batches.) The flask was placed in a high pressure reactor. The reactor was charged with H₂ and purged for 5 cycles and was finally pressurized with H₂ at 4 bars (60 psi). After stirring at rt for 3 h, the reaction mixture was filtered through Celite, and the solvent removed in vacuo. The resulting residue was dissolved in water (5 mL) and the solution neutralized with 2.5N NaOH (pH 11.5), and extracted with Et₂O (3×50 mL). The combined organic layers were dried (Na₂SO₄), filtered and concentrated in vacuo to yield 1.165 g (95%) of the crude title compound as slightly yellow crystals. mp 36.0-36.5° C. ¹H NMR (300 MHz, CDCl₃) δ 7.30-7.23 (m, 2H), 7.18-7.11 (m, 2H), 1.86 (bs, 1H), 1.48 (s, 12H).

### 1,1,3,3-Tetramethylisoindolin-2-yloxyl (18)

To a solution of the amine 17 (1.46 g, 8.33 mmol) in a 14:1 mixture of MeOH/MeCN (16.6 mL) were added successively NaHCO₃ (560 mg, 6.67 mmol), Na₂WO₄.2H₂O (83.3 mg, 0.25 mmol) and 30% aq. H₂O₂ (3.12 mL, 27.50 mmol). The resulting suspension was stirred at rt. After 18 h, a bright yellow suspension formed and 30% aq. H₂O₂ (3.00 mL, 26.44 mmol) was added. The reaction mixture was stirred for 2 days, then diluted with water and extracted with hexanes (2×). The combined organic layers were washed with 1M H₂SO₄ and brine, dried (Na₂SO₄), filtered and concentrated in vacuo to yield 1.55 g (98% crude) of the title compound as a yellow crystalline powder that was used for the next step without further purification. mp 122-125° C. (softening point: 108° C.); HRMS (EI) m/z calcd for C₁₂H₁₇NO 191.1310. found 191.1306.

### 5-Nitro-1,1,3,3-tetramethylisoindolin-2-yloxyl (19)

Conc. H₂SO₄ (13.5 mL) was added dropwise to 18 (1.345 g, 7.07 mmol) cooled in an ice-water bath, forming a dark-red solution which was then warmed to 60° C. for 15 min and then cooled to 0° C. Conc. HNO₃ (0.90 mL, 19.09 mmol) was added dropwise. When the reaction appeared complete, the yellow-orange solution was heated at 100° C. for 10 min, the color turning to red-orange. After cooling to rt, the reaction mixture was neutralized by careful addition to ice-cooled 2.5N NaOH (30 mL). This aqueous phase was extracted with Et₂O until it became colorless and the combined organic layers were dried (Na₂SO₄), filtered and concentrated in vacuo to yield 1.64 g (98%) of the crude title compound as a yellow-orange powder, that was used for the next step without further purification.

### 5-amino-1,1,3,3-tetramethylisoindolin-2-yloxyl (5-amino-TMIO)

A flask containing a solution of 19 (1.50 g, 6.38 mmol, crude) in MeOH (75 mL) was purged and filled with argon, then 10% Pd/C (150 mg) was added. The flask was purged and filled 3 times with H₂, and the resulting black suspension was stirred at rt under H₂ (1 atm) for 4 h. The reaction mixture was then filtered through Celite, the Celite washed with MeOH, and the solution concentrated in vacuo to yield 1.38 g of the crude title compound as a yellow solid, that was used for the next step without further purification. ¹H NMR (300 MHz, CD₃OD) δ 6.89 (d, 1H, J=8.1 Hz), 6.25 (dd, 1H, J=8.1, 2.1 Hz), 6.54 (d, 1H, J=2.1 Hz), 3.35 (s, 2H), 1.34 (appd, 12H, J=5.7 Hz).

To a solution of the crude hydroxylamine (1.38 g, 6.38 mmol) in MeOH (75 mL) was added Cu(OAc)₂.H₂O (26 mg, 0.128 mmol). The reaction mixture was stirred at rt under air for 1.5 h, the color turning to dark brown. The solvent was then removed in vacuo, the residue taken up in CHCl₃ and a small amount of MeOH to dissolve the insoluble material, and washed with water. The aqueous phase was extracted twice with CHCl₃, and the combined organic layers were washed with brine, dried (Na₂SO₄), filtered and concentrated in vacuo. Flash chromatography (SiO₂, 6:4 to 5:5, hexanes/EtOAc) afforded 1.126 g (86%) of the title compound as a yellow powder. mp 192-194° C. (softening point: 189° C.); HRMS (EI) m/z calcd for C₁₂H₁₇N₂O 205.1341. found 205.1336.

### TMIO-5-yl-(S,E)-5-(tert-butoxycarbonylamino)-7-methyloct-3-enamide (20)

To a solution of the alcohol 9a (187 mg, 0.728 mmol, prepared according to previous examples) in acetone (7 mL) at 0° C. was slowly added a solution of Jones reagent (2.5M, 0.73 mL, 1.821 mmol). The resulting dark suspension was stirred at 0° C. for 1 h, then diluted with Et₂O and water. The aqueous phase was separated and extracted with Et₂O (2×). The combined organic layers were washed with water (2×) and brine (1×), dried (Na₂SO₄), filtered and concentrated in vacuo to yield 190 mg (96%) of the crude title compound as a slightly yellow oil, that was used for the next step without further purification.

To a solution of this acid (187.4 mg, 0.691 mmol, crude) in dry DCM (8 mL) at 0° C. were added successively 5-amino-TMIO (212.6 mg, 1.036 mmol), DMAP (93.7 mg, 0.760 mmol), HOBt.H₂O (102.6 mg, 0.760 mmol) and EDCI (162.1 mg, 0.829 mmol). The resulting yellowish solution was stirred at rt under argon for 16 h, and then washed with sat. NH₄Cl. The aqueous phase was separated and extracted once with DCM, and the combined organic layers were washed twice with 1N HCl and once with sat. NaHCO₃, dried (Na₂SO₄), filtered and concentrated in vacuo. Flash chromatography (SiO₂, 6:4, hexanes/EtOAc) afforded 221.0 mg (70%) of the title compound as a pale orange foam. mp 78-79° C. (softening point: 70° C.); [α]_{D}²²+72.2 (c 0.5, DCM); ESIMS m/z 481 ([M+Na]⁺, 50), 939 ([2M+Na]⁺, 100).

Compound 6-amino-1-methyl 2-azaadamantane N-oxyl (6-amino-1-Me-AZADO) and (30) are shown in FIG. 12 and were prepared according to the following.

### 2-Adamantanecarbonitrile (tricyclo[3.3.1.13,7]decane-2-carbonitrile, 22)

A 3-5° C. solution of 2-adamantanone (tricyclo[3.3.1.13,7]decan-2-one, 21) (21.0 g, 137 mmol),p-tolylsulfonylmethyl isocyanide (TosMIC, 35.5 g, 178 mmol) and EtOH (14 mL, 233 mmol) in 1,2-dimethoxyethane (DME, 470 mL) was treated with portionwise addition of solid t-BuOK (39.2 g, 342 mmol), maintaining the internal temperature below 10° C. After the addition, the resulting slurry reaction mixture was stirred at rt for 30 min and then at 35-40° C. for 30 min. The heterogeneous reaction mixture was filtered and the solid washed with DME. The filtrate was concentrated in vacuo, loaded to a short Al₂O₃ column (activated, neutral, Brockmann I, 150 mesh, 7 cm thick×15 cm height), and washed off with a 5:1 mixture of hexanes/DCM (˜1.5 L). The solution was concentrated in vacuo to afford 19.0 g (86%) of the title compound as a white powder. ¹H NMR (300 MHz, CDCl₃) δ 2.91 (s, 1H), 2.23-2.08 (in, 4H), 2.00-1.80 (in, 4H), 1.80-1.66 (m, 6H).

### 2-Adamantane carboxylic acid (23)

A mixture of the nitrile 22 (18.9 g, 117 mmol) in AcOH (56 mL) and 48% HBr (224 mL) was stirred at 120° C. overnight. The reaction mixture was cooled at 4° C., standing for 4 h, then filtered. The solid was washed with water and dried in vacuum over silica gel overnight, to yield 20.6 g (98%) of the title compound as off-white crystals. ¹H NMR (300 MHz, DMSO-d₆) δ 12.09 (s, 1H), 2.55-2.47 (m, 1H), 2.20 (bs, 2H), 1.87-1.64 (in, 10H), 1.60-1.50 (in, 2H).

### 5,7-Dibromo-2-adamantane carboxylic acid (24)

A vigorously stirred 0° C. solution of AlBr₃ (18.9 g, 69.6 mmol), BBr₃ (2.40 g, 9.49 mmol) and Br₂ (40 mL) was treated portionwise with the acid 23 (5.70 g, 31.6 mmol). Upon completion of the addition, the reaction mixture was stirred at 70° C. for 48 h, then cooled in an ice bath, and quenched carefully with sat. sodium bisulfate. Stirring was continued at rt overnight. The resultant pale brown suspension was filtered, the solid washed with water and dried overnight under vacuum at 60° C. to yield 10.95 g (quant.) of the crude title compound as a beige powder. ¹H NMR (300 MHz, DMSO-d₆) δ 12.56 (bs, 0.3H), 2.85 (appd, 2H, =12.9 Hz), 2.75-2.55 (m, 2H), 2.50-2.35 (m, 2H), 2.35-2.10 (m, 7H).

### (5,7-Dibromo-adamantan-2-yl)-carbamic acid tert-butyl ester (25)

A suspension of the acid 24 (2.00 g, 5.92 mmol) in dry toluene (30 mL) was treated successively with Et₃N (1.0 mL, 7.10 mmol) and diphenylphosphoryl azide (DPPA, 1.6 mL, 7.10 mmol). The resulting mixture was stirred at 85° C. for 15 h. To a separated flask containing a solution of t-BuOK (1.35 g, 11.8 mmol) in dry THF (80 mL) at 0° C. was added the isocyanate solution dropwise via a dropping funnel. The resulting reaction mixture was allowed to warm to rt over 30 min, and then it was quenched with water. The THF was removed in vacuo, and the resulting material was diluted with EtOAc. The organic layer was washed with 1N HCl, sat. NaHCO₃ and brine, dried (Na₂SO₄), filtered and concentrated in vacuo. Flash chromatography (SiO₂, 95:5 to 8:2, hexanes/EtOAc) afforded 1.20 g (50%, 2 steps) of the title compound as a white powder. ¹H NMR (300 MHz, CDCl₃) δ 4.68 (bs, 1H), 3.76 (bs, 1H), 2.87 (s, 2H), 2.47-2.13 (m, 10H), 1.46 (s, 9H).

### (7-Methylene-bicyclo[3.3.1]nonan-3-one-9-yl)-carbamic acid tert-butyl ester (26)

A solution of 25 (125 mg, 0.305 mmol) in dioxane (0.80 mL) was treated with 2N NaOH (0.70 mL, 1.37 mmol) and irradiated under microwaves (μw, Biotage) for 15 min at 180° C. The dioxane was removed in vacuo. The residue was dissolved in DCM, washed with water, dried (Na₂SO₄), filtered and concentrated in vacuo to afford 82.5 mg of crude 9-amino-7-methylene-bicyclo[3.3.1]nonan-3-one as a yellow oil, that was used for the next step without further purification.

To a solution of this crude amine in dry DCM (5 mL) was added Et₃N (0.13 mL, 0.913 mmol) and then Boc₂O (73.8 mg, 0.335 mmol) at 0° C. The reaction mixture was stirred at rt under N₂ for 14 h. The reaction was quenched with sat. aq. NH₄Cl and the aqueous phase extracted twice with DCM. The combined organic layers were dried (Na₂SO₄), filtered and concentrated in vacuo. Flash chromatography (SiO₂, 7:3, hexanes/EtOAc) afforded 48.0 mg (59%, 2 steps) of the title compound as a white powder. ¹H NMR (300 MHz, CDCl₃) δ 4.93 (bs, 0.25H), 4.84 (s, 2H), 4.81 (bs, 0.75H), 4.12 (bs, 0.25H), 3.91 (appbd, 0.75H, J=3.6 Hz), 2.64-2.37 (m, 6H), 2.37-2.23 (in, 3.25H), 2.17 (appbd, 0.75H, J=13.8 Hz), 1.48 and 1.46 (2 s, 9H).

### (7-Methylene-bicyclo[3.3.1]nonan-3-one oxime-9-yl)-carbamic acid tert-butyl ester (27)

To a solution of ketone 26 (137 mg, 0.515 mmol) in dry pyridine (1 mL) was added NH₂OH.HCl (109 mg, 1.54 mmol). The reaction mixture was stirred at room temperature under argon for 23 h. The solvent was then removed in vacuo, and the residue was diluted with EtOAc and then water was added. The layers were separated and the aqueous phase extracted with EtOAc. The combined organic layers were washed with 5% aq. CuSO₄ (3×), brine (1×), dried (Na₂SO₄), filtered and concentrated in vacuo. Flash chromatography (SiO₂, 4:6, hexanes/EtOAc) afforded 133 mg (92%) of the title compound as a colorless gum. ¹H NMR (300 MHz, CDCl₃) δ 7.02 (bs, 0.6H), 4.90 (bs, 0.25H), 4.80 (d, 1H, J=2.1 Hz), 4.76 (bs, 0.75H), 4.69 (d, 1H, J=2.1 Hz), 3.87 (bs, 1H), 3.26 (d, 0.25H, J=16.8 Hz), 3.11 (d, 0.75H, J=16.8 Hz), 2.55-2.48 (m, 4H), 2.48-2.20 (m, 4H), 2.16 (appd, 0.25H, J=17.1 Hz), 2.04 (dd, 0.75H, J=17.1, 5.4 Hz), 1.47 (s, 9H).

### (1-Iodomethyl-2-azaadamantan-6-yl)-carbamic acid tert-butyl ester (28)

To a mixture of oxime 27 (130 mg, 0.464 mmol) and MoO₃ (94 mg, 0.649 mmol) in dry MeOH (4.6 mL) at 0° C. under argon was added NaBH₄ (179 mg, 4.64 mmol) portionwise. The reaction mixture was stirred at 0° C., and 2 additional amounts of NaBH₄ (179 mg, 4.64 mmol) were added portionwise after 2.5 h and after 5.5 h. After 7 h, the dark brown reaction mixture was quenched with acetone and then filtered through Celite, and the Celite rinsed with acetone. The filtrate was concentrated in vacuo. The resulting residue was diluted with water and extracted twice with EtOAc. The combined organic layers were washed with brine, dried (K₂CO₃), filtered and concentrated in vacuo to afford 136 mg of the crude amine as a yellow oil, that was used for the next step without further purification.

To a suspension of this crude amine in dry acetonitrile (MeCN, 2.3 mL) at 0° C. under argon was added I₂ (117 mg, 0.462 mmol). The reaction mixture was allowed to stir at room temperature for 4 h and then quenched with sat. NaHCO₃ and sat. Na₂S₂O₃. The resulting mixture was extracted twice with DCM/CHCl₃, and the organic layer was dried (K₂CO₃), filtered and concentrated in vacuo. Flash chromatography (SiO₂, 95:5 to 9:1, DCM/MeOH) afforded 76.5 mg (42%) of the title compound as a brown oil. ¹H NMR (300 MHz, CDCl₃) δ 4.83 (bs, 1H), 3.77 (bs, 1H), 3.30 (bs, 1H), 3.24 (apps, 2H), 2.14 (appbs, 2H), 1.94 (appbd, 2H, J=13.5 Hz), 1.75 (m, 6H), 1.46 (s, 9H).

### (1-Methyl-2-azaadamantane-N-oxyl-6-yl)-carbamic acid tert-butyl ester (29)

Deiodination of the amine 28 can be achieved by treating 28 with a reducing agent, such as LiAlH₄ or NaBH₄, possibly in the presence of a catalyst, such as InCl₃, and in a polar aprotic solvent such as THF or MeCN. Oxidation of the resulting amine to afford the corresponding nitroxide 29 can be achieved by treating the said amine with H₂O₂ in the presence of a catalytic amount of Na₂WO₄.2H₂O, in a solvent mixture of MeOH and H₂O.

### 6-Amino-1-methyl-2-azaadamantane-N-oxyl (6-amino-1-Me-AZADO)

Cleavage of the Boc-protecting group can be achieved by treating the protected amine 29 with trifluoroacetic acid (TFA) in DCM, to afford the free amine 6-amino-1-Me-AZADO.

### (1-Me-AZADO-6-yl)-(S,E)-5-(tert-butoxycarbonylamino)-7-methyloct-3-enamide (30)

Jones oxidation of (S,E)-tert-butyl 8-hydroxy-2-methyloct-5-en-4-ylcarbamate (9a) affords the corresponding acid as described above. Compound (9a) is prepared according to previous examples. Amide coupling of the said acid with 6-amino-1-Me-AZADO is achieved following the conditions described above, using the coupling agents EDCI, DMAP, and HOBt-hydrate in CH₂Cl₂ (DCM), to yield compound (30).

### Example 7

### Intraesophageal Administration of GS-Nitroxide (JP4-039) Protects Against Ionizing Irradiation-Induced Esophagitis

Preparation of JP4-039 in F-15 Formulation.

The GS-nitroxide JP4-039 was formulated at a final drug concentration of 8 mg/nil in cationic mutilamellar liposomes termed F-15. F-15 is a unique form of multilamellar liposomes containing diacylphosphatidyl choline from soybean, Tween 80 and a cationic lipid, N,N-di oleylamine amido-L-glutamate. JP4-039 was entrapped between lipid bilayers which allows improved dispersibility/solubility and slow release over time of the drug from the liposome particles. In addition, N,N-di oleylamine amido-L-glutamate provides positive surface charges in order to facilitate adherence of the liposomes loaded with the drug to the esophageal mucosa. Its composition was: soy PC:Tween-80:N,N-di oleylamine amido-L-glutamate (4:1:1 w/w) with a final drug concentration of 8 mg/ml in PBS Unlike most known cationic liposome formulations, it has low toxicity to cultured mammalian cells (>0.5 mg/ml).

Soy phosphatidyl choline, Lissamine rhodamine-phosphatidylethanolamine were obtained from Avanti Polar Lipids (Alabaster, Ala., USA); Tween-80, tert-boc-L-glutamic acid, oleylamine, dicyclohexylcarbodiimide, N-hydroxysuccinimide, trifluoroacidic acid were obtained from Sigma-Aldrich (St. Louis, Mo., USA). Dubecco's phospate buffered saline (d-PBS) was obtained from Lonza (Walkersville, Md., USA). A cationic lipid, L-glutamic acid-1,5,-dioleyl amide [NH₂-L-Glu(NHC₁₈H₃₆)₂] was synthesized using a modified route as previously described (Lee K C, et al. Formation of high axial ratio microstructures from peptides modified with glutamic acid dialkyl amides. Biochimica Biophysica Acta 1371: 168-184, 1998), by coupling t-Boc-L-glutamic acid and oleylamine with dicyclohexylcarbodiimide and N-hydroxysuccinimide as the coupling agents, followed by use of trifluoroacidic acid as the deprotecting agent.

The lipid mixture (6 mg) and drug to be encapsulated (1 mg) were dissolved in 100 μl tert-butanol, frozen on dry ice, and lyophilized overnight into a cake. The next day, a 62.5 μl d-PBS was added to the lipid cake and allowed to hydrate for 24 h at room temperature. Cationic liposomes were prepared from the lipid suspension by manual homogenization using a pair of custom-made tight-fit tube and pestle until a homogeneous consistency were reached. Finally, the liposome suspension was removed from the tube and another 62.5 μl d-PBS was used to rinse the tube and pestle and the wash solution were combined with the liposome suspension. Thus, 1 mg JP4-039 was formulated in 225 μl volumes. The final particle sizes were measured by a laser dynamic scattering method (NP-4 Particle Sizer, Beckman Coutler, Inc., Brea, Calif., USA) and found to be in the range of 200-300 nm with a mean of ˜255 nm in diameter. Each mouse received an intraesophageal injection of 110 μs of F15 formulation containing 400 μg JP4-039. To determine whether Tween-80 was required for effective uptake, an identical formulation without Tween-80 was tested.

Animals and animal care. C57BL/6HNsd female and C57BL/6JHNsd-GFP male mice (22-22 gm) were housed, five per cage and fed standard laboratory chow according to previous publications (3). C57BL/6NHsd mice (15 per group) were irradiated (details are given in the next section) and received swallowed JP4-039 or MnSOD-PL pre- or post-radiation. The mice were monitored for development of esophagitis.

Irradiation. Mice were irradiated to 28 or 29 Gy to the upper body using a JL Shepherd Mark I cesium irradiator (J. L. Shepherd and Associates, San Fernando, Ca, USA) (70 cGy/mm), according to published methods (Epperly M W, et al. Zhang X, Nie S, Cao S, Kagan V, Tyurin V and Greenberger J S: MnSOD-plasmid liposome gene therapy effects on ionizing irradiation induced lipid peroxidation of the esophagus. In Vivo 19: 997-1004, 2005). The head and abdomen were shielded, as described previously (Stickle R L, et al. Prevention of irradiation-induced esophagitis by plasmid/liposome delivery of the human manganese superoxide dismutase (MnSOD) transgene. Radiat Oncol Invest Clinical & Basic Res 7: 204-217, 1999), so that only the thoracic cavity received irradiation.

Intraesophageal drug administration. The methods for preparation of MnSOD-plasmid liposomes using PNVL3 lipid have been published previously (Epperly M W, et al. Modulation of radiation-induced cytokine elevation associated with esophagitis and esophageal stricture by manganese superoxide dismutase-plasmid/liposome (MnSOD-PL) gene therapy. Radiat Res 155: 2-14, 2001). Briefly, 100 μl liposomes containing 100 mg plasmid were injected by syringe intraesophageally into non-anesthetized mice immediately after they received 100 μl distilled water.

Measurement of JP4-039 nitroxide in serum and in tissues by electron paramagnetic resonance. Electron paramagnetic resonance (EPR) spectra of nitroxide radicals in cells or in mitochondrial fractions were recorded after mixing with acetonitrile (1:1 v/v) after 5 min incubation with 2 mM K₃Fe(CN)₆ using a JEOL-RE1XEPR spectrometer (JEOL, USA, Inc., Peabody, Mass., USA) under the following conditions: 3350 G center field, 25 G scan range, 0.79 G field modulation, 20 mW microwave power, 0.1 s time constant, 4 min scan time. Under these experimental conditions, nitroxides were not detectably oxidized by K₃F₃(CN)₆ to EPR-silent oxoamminium cations. Mitochondria-enriched fractions were obtained by differential centrifugation. Briefly, cells were suspended in a mitochondria isolation buffer (210 mM mannitol, 70 mM sucrose, 10 mM Hepes-KOH, pH 7.4, 1 mM EDTA, 0.1% BST and cocktail protease inhibitor) and disrupted by Dounce homogenization. Unbroken cells, nuclei, and debris were removed by 10 min centrifugation at 700 g at 4° C. Mitochondria-rich fractions were obtained by 10 min centrifugation at 5,000 g and washed twice with an isolation buffer. Partitioning efficiency was calculated as a percentage of the initial signal. The amounts of nitroxide radicals integrated into mitochondria were normalized to the content of cytochrome c oxidase subunit IV. For nitroxide integration in whole cells, tissue, or isolated mitochondria, tissue or mitochondria (1 μg/μl) were incubated with 10 μM nitroxides in an incubation buffer (210 mM sucrose, 10 mM Hepes-KOH, pH 7.4, 70 mM KCI, 0.5 mM EGTA, 3 mM phosphate) for 15 mM at room temperature in the presence or absence of 5 mM succinate. After that, samples were centrifuged at 10,000 g for 5 min, and the pellets washed twice with the incubation buffer and analyzed by EPR as described previously (Borisenko G G, et al. Nitroxides scavenge myeloperoxidase-catalyzed thiyl radicals in model systems and in cells. J Am Chem Soc 4(30): 9221-9232, 2004 and Jiang J, et al. A mitochondria-targeted triphenylphosphonium-conjugated nitroxide functions as a radioprotector/initigator. Radiat Res 172(6): 706-717, 200938-39).

Bone marrow transplantation, esophageal excision and cell sorting. Five days after irradiation, the time shown to optimize marrow cell homing to the irradiated esophagus, mice received intravenous injection of 1.0×10⁷ C57BL/6HNsd GFP+ male bone marrow cells prepared as single-cell suspension from donor male mice according to published methods (Niu Y, et al. Irradiated esophageal cells are protected from radiation-induced recombination by MnSOD gene therapy. Rad Res 173: 453-461, 2010 and Niu Y, et al. Intraesophageal MnSOD-plasmid liposome administration enhances engraftment and self-renewal capacity of bone marrow derived progenitors of esophageal squamous epithelium. Gene Therapy 15: 347-356, 2008). At serial time points after marrow transplantation, esophagus specimens were removed, and single cell suspensions prepared according to published methods (Id.). The esophagus cell suspensions were sorted for GFP+ cells. The number of GFP+ cells per 10⁶ was calculated as described previously (Id.). (GFP+) cells were placed on slides, and stained for detection of donor cell markers (Id.).

Statistics. In vitro data analysis and estimation of survival of mice were performed using published statistical methods (Epperly M W, et al. Radiat Res 155: 2-14, 2001). The Kruskal-Wallis test and post-hoc Mann-Whitney test were used to evaluate donor marrow cells in the esophagus as described (Niu Y, et al. Rad Res 173: 453-461, 2010). A SAS statistical program was used to perform the statistical analysis (SAS Institute, Cary, N.C., USA).

### Results

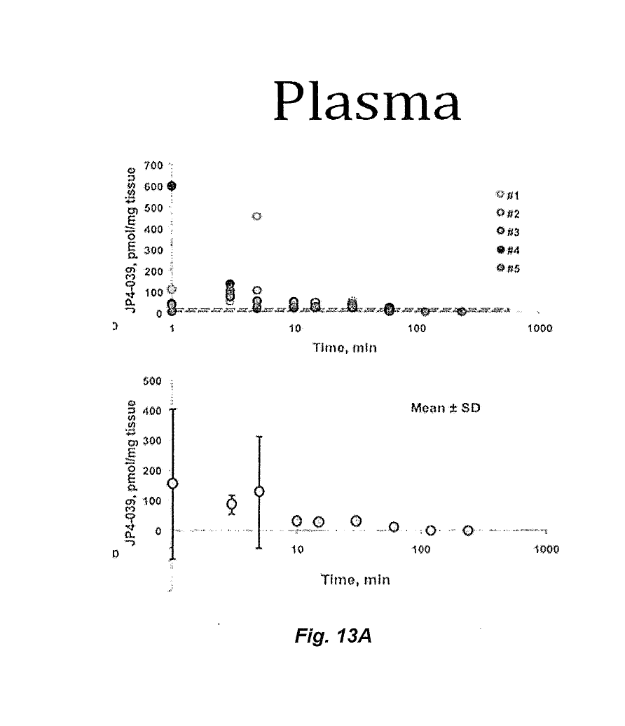

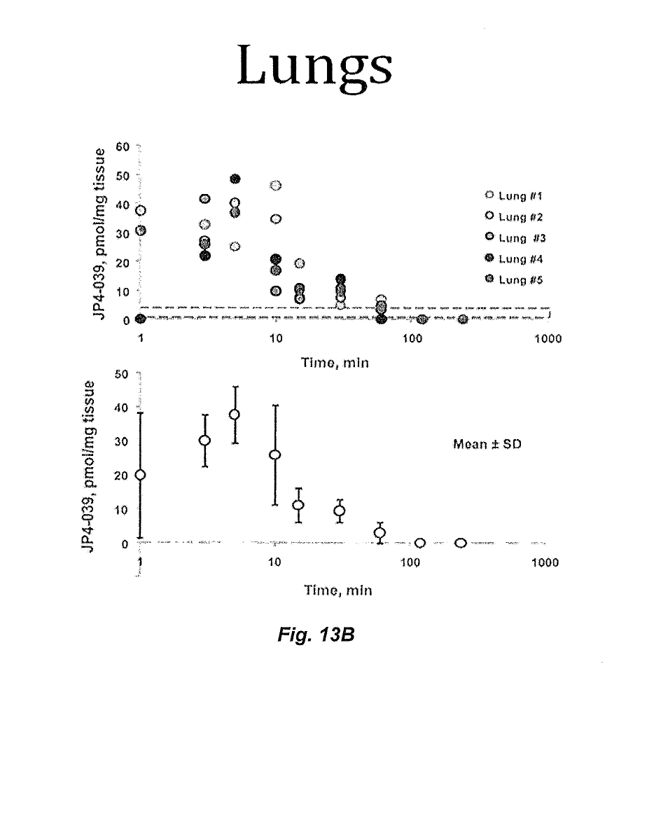

Intravenous JP4-039 systemic pharmacokinetics and intraesophageal formulation mediated delivery to esophagus. First, the clearance of JP4-039 from plasma was tested after the intravenous injection of 10 mg/kg JP4-039 in 100 μl volumes of diluents (FIG. 13) using EPR measurements. JP4-039 was cleared from plasma by 10 min, but was detected in lung (and intestine) for over 30 minutes.

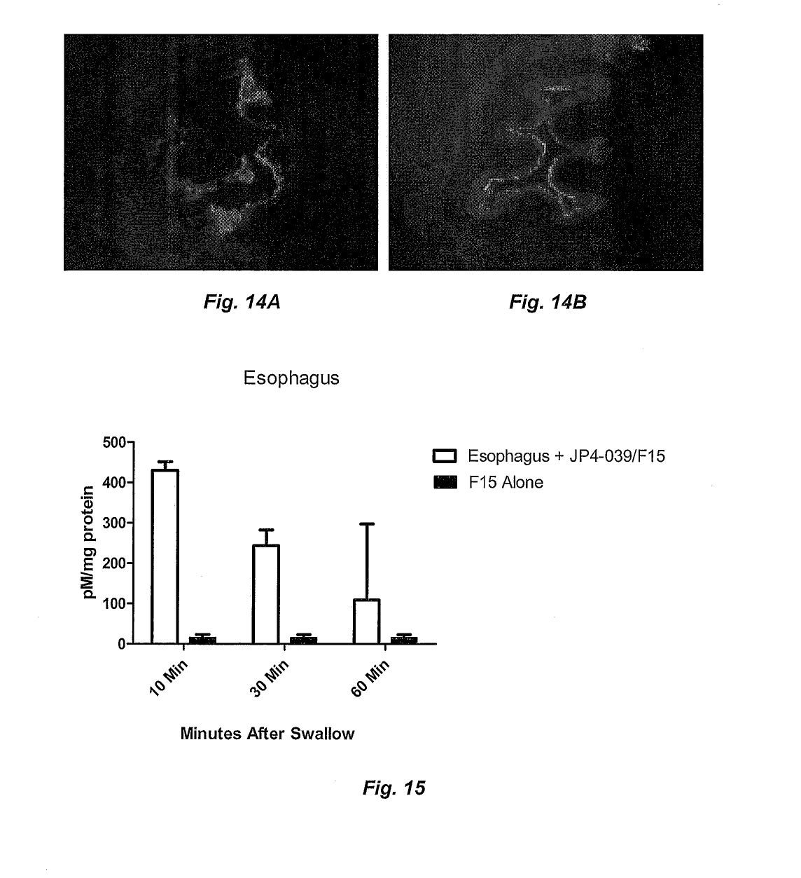

Intraesophageal administration of 0.5 mole percent of Lissamine Rhodamine B-DOPE, a red phycoerythrin dye, by control multilamellar liposomes without dioleoylamindo-L-glutamate compared to the F15 formulation was next carried out. F15 emulsion containing Tween-80 was superior to the control formulation (FIG. 14). Next, the nitroxide signal of JP4-039 in the esophagus was measured in vivo after giving JP4-039/F15 by swallow. The nitroxide signal, as detected by EPR in esophagus explants existed for up to 60 min after swallow (FIG. 15).

Esophageal administration of JP4-039/F15 formulation improves survival of thoracic-irradiated mice. Groups of mice received JP4-039/F15, or F15 formulation alone, then 10 min later 28 Gy to the thoracic cavity and were then followed for survival. Subgroups receiving MnSOD-PL or JP4-039 in F15 formulation showed a significant increase in survival compared to mice receiving F15 formulation alone (FIG. 16). Survival was improved significantly but was not sustained as with mice receiving MnSOD-PL 24 hours prior to irradiation (FIG. 16).

Intraesophageal JP4-039/formulation improves survival through recovery of endogenous esophageal progenitor cells. To determine whether esophageal radioprotection by JP4-039 may be increased by facilitating migration to the esophagus of bone marrow-derived cells, experimental methods were used, which previously demonstrated the bone marrow origin of progenitors of esophageal squamous epithelium (22-23). Five groups of 15 mice each received 29 Gy to the upper body. One group received MnSOD-PL 24 hours prior to irradiation. Two groups received JP4-039/F15 formulation either 10 min prior to irradiation or JP4-039/F15 immediately after irradiation.

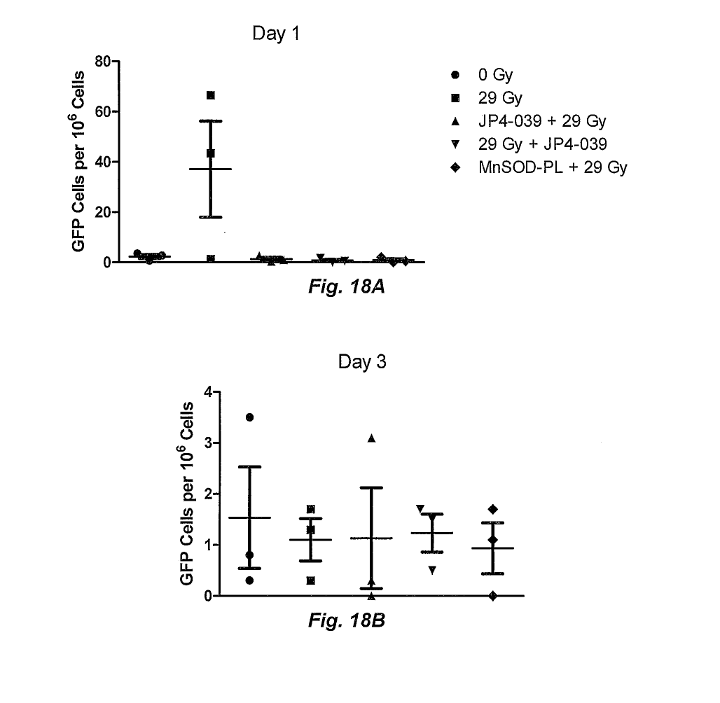

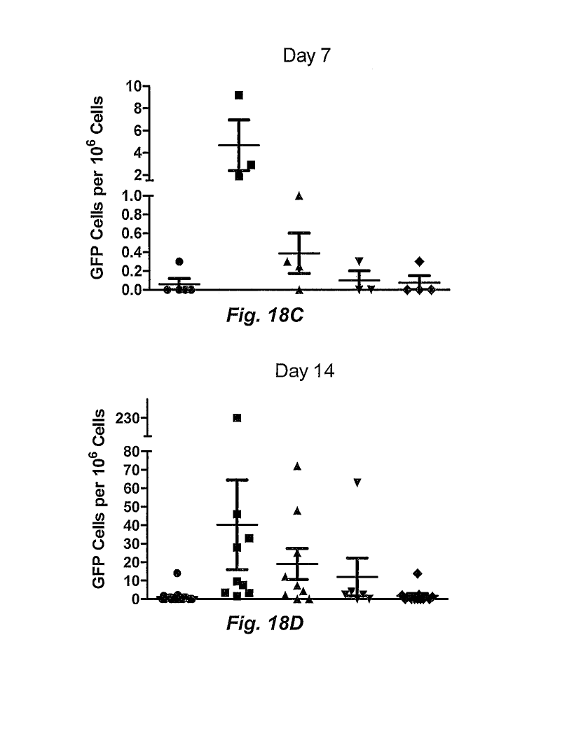

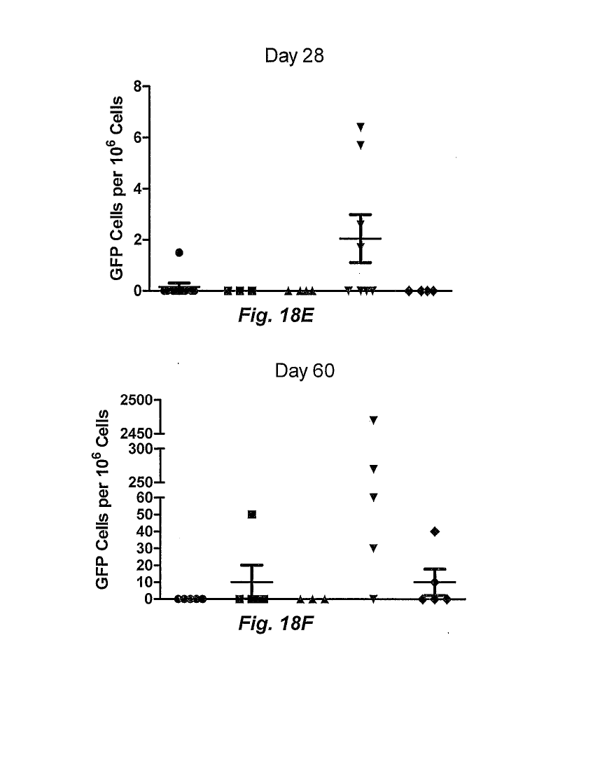

All mice received GFP+ male marrow injected intravenously at day five after irradiation. Mice receiving either MnSOD-PL or JP4-039 before irradiation showed improved survival (FIG. 17). Furthermore, the survival of the JP4-039/F15 group was sustained compared to the MnSOD-PL group. Mice that received 1×10⁷ GFP+ bone marrow cells intravenously five days after irradiation survived at a 30% level by being given bone marrow (FIG. 17); an improvement over mice without bone marrow donation (FIG. 16). At time points including 1, 3, 7, 14, 28, and 60 days after bone marrow injection, esophagus samples were removed from subgroups of mice, and single cell suspensions sorted for the number of GFP+ cells. At days 1 and 3, five esophagi were pooled for sorting of GFP+ cells. At later days, each esophagus was kept separate. As shown in FIG. 18, GFP+ cells were detected in some esophagus samples at all time points. There were low numbers at days 1, 3, 7, and 28. At days 14 and 60, individual mice had high numbers of GFP+ cells, but there was significant variation between mice. These results confirm and extend those in previous publications demonstrating that bone marrow-derived progenitors of esophageal squamous epithelium migrate into the irradiated esophagus and persist out to 60 days after irradiation, prominent in the 29 Gy+JP4-039 group (Table A).

TABLE A
Median and inter-quartile range (in parentheses) for the number of GFP⁺ cells per 10⁶ cells
in the esophagus of mice in each of the treatment groups at each day of measurement.

| | Treatment | | | | |
|---|---|---|---|---|---|
| | | | JP4-039 + 29 | 29 Gy + JP4- | MnSOD-PL + 29 |
| Day | 0 Gy | 29 Gy | Gy | 039 | Gy |
| 1 | 2.7 (0.6-3.5) | 43.3 (1.3-66.6) | 0.9 (0.3-2.6) | 0.5 (0-1.6) | 0.5 (0-2) |
| | N = 3 | N = 3 | N = 3 | N = 3 | N = 3 |
| 3 | 0.8 (0.3-3.5) | 1.3 (0.3-1.7) | 0.3 (0-3.1) | 1.5 (0.5-1.7) | 1.1 (0-1.7) |
| | N = 3 | N = 3 | N = 3 | N = 3 | N = 3 |
| 7 | 0 (0-0) | 2.9 (1.9-9.2) | 0.28 (0.13-0.65) | 0 (0-0.3) | 0 (0-0.15) |
| | N = 5 | N = 3 | N = 4 | N = 3 | N = 4 |
| | | p₁ = 0.018* | | | |
| 14 | 0 (0-0) | 9.6 (3.5-33) | 7.3 (2.1-25) | 2.35 (0-3.9) | 0 (0-2) |
| | N = 15 | N = 9 | N = 9 | N = 6 | N = 11 |
| | | p₁ < 0.0001 | p₁ = 0.0028 | p₁ = 0.017 | p₂ < 0.0001** |
| 28 | 0 (0-0) | 0 (0-0) | 0 (0-0) | 0.85 (0-4.15) | 0 (0-0) |
| | N = 10 | N = 3 | N = 4 | N = 8 | N = 4 |
| 60 | 0 (0-0) | 0 (0-0) | 0 (0-0) | 60 (30-270) | 0 (0-0) |
| | N = 5 | N = 5 | N = 3 | N = 5 | N = 5 |
| | | | | p₁ = 0.048 | |

* p₁ is the p-value for the comparison with 0 Gy group using Mann-Whitney U-test;
** p₂ is the p-value for the comparison with 29 Gy group using Mann-Whitney U-test.

Statistical evaluation was next carried out. At days 1, 3 and 28, the Kruskal-Wallis test showed p-values 0.18, 0.94, and 0.060 respectively, indicating that all these groups had an equal number of GFP cells at these days (Table A). At day 7, the Kruskal-Wallis test showed a p-value of 0.035, indicating that these groups did not have equal number of GFP cells. The post-hoc Mann-Whitney test revealed that the 29 Gy group had a significantly higher number of GFP cells than the 0 Gy group (p=0.018). At day 14, the Kruskal-Wallis test showed a p-value of 0.0002, indicating that these groups did not have equal numbers of GFP cells. The post-hoc Mann-Whitney test revealed that each of the 29 Gy, JP4-039+29 Gy, and 29 Gy+JP4-039 groups had significantly higher numbers than the 0 Gy group (p<0.0001, p=0.0028 and p=0.017, respectively). The MnSOD-PL+29 Gy group had a significantly lower number than the 29 Gy group (p<0.0001). The results at day 60 showed a persistent increase in donor marrow-derived cells in the 29 Gy+JP4-039 group. At day 60, the Kruskal-Wallis test showed a p-value of 0.035, indicating that these groups did not have equal number of GFP cells. The post-hoc Mann-Whitney test revealed that the 29 Gy+JP4-039 group had a significantly higher number than the 0 Gy group (p=0.048).

### Discussion

A mitochondrial targeted 4-AT derivative, JP4-039 (in which mitochondrial localization is achieved by linkage of the active nitroxide molecule to a peptide isostere, based on a mitochondrial targeting segment of the cyclopeptide antibiotic Gramicidin-S) is a highly effective radiation protector and mitigator in vitro and in vivo. To determine whether organ-specific radioprotection was achievable using JP4-039, this study developed a novel formulation (F15) and JP4-039 was dispersed in this formulation for intra-esophageal (swallowed) administration. Delivery of JP4-039/F15 intraesophageally before or after thoracic irradiation provided significant protection of the esophagus and improved survival. These results establish that a small molecule, mitochondrial-targeted nitroxide, can be an effective esophageal radioprotector.

The present results demonstrated that esophageal radioprotection is mediated in large part by protection of endogenous esophageal progenitor cells, with minimal contribution of bone marrow-derived progenitors of esophageal epithelium: The observation that higher numbers of donor marrow cells were detected in the explanted esophagi of the same mice at days 7, 14 and 60 after transplant may reflect the growth and expansion of rare foci of single marrow-derived esophageal cells into discrete foci as described previously (Niu Y, et al. Rad Res 173: 453-461, 201010; Epperly M W, et al. Bone marrow origin of cells with capacity for homing and differentiation to esophageal squamous epithelium. Rad Res 162: 233-240, 2004; and Niu Y, et al. Gene Therapy 15: 347-356, 2008, 22, 23). Whether these foci are derived from rare stem cells growing in rare niches is not yet known.

Previous studies showed enhanced migration to the irradiated esophagus of bone marrow-derived progenitors of esophageal squamous epithelium in mice receiving MnSOD-PL 24 hours prior to irradiation (Epperly M W, et al. Rad Res 162: 233-240, 2004 and Niu Y, et al. Gene Therapy 15: 347-356, 2008). The difference between the MnSOD-PL-mediated radioprotection and that mediated by the small molecule JP4-039 is not yet known, but a low-level contribution of bone marrow-derived progenitors was detected in the esophagi of mice treated by each agent. The best survival of GFP+ cells at day 60 was with JP4-039 delivered after irradiation. Swallowed MnSOD-PL-treated mice may have experienced persistent gene product protection and may have effectively protected true stem cells and their niches. Therefore, irradiation protection by MnSOD-PL may have been greater, allowing for homing of only bone marrow-derived short-term repopulating progenitors. In contrast, intraesophageal injected JP4-039 may have reached both esophageal stem cells and their microenvironment, but cleared rapidly. While JP4-039 may have prevented quiescent stem cell apoptosis, reduced stromal microenvironmental protection due to more rapid drug clearance may have allowed irradiation killing of more primitive esophageal stem cells and facilitated homing of bone marrow-derived primitive progenitors that protected and persisted to day 60.

### Example 8

### Amelioriation of Radiation Esophagitis by Orally Administered GS-Nitroxide

JP4-039 was formulated at 8 mg/ml in F15 formulation as described herein. The final product had a concentration of 1 mg of JP4-039. Adult female C57BL/6HNsd mice (20-25 g) received 100 of distilled water intraesophageally via feeding tube followed by 100 μl of F15 alone, MnSOD-PL or JP4-039 prior to irradiation (described below). The stock solutions described above were diluted, so that the total amount of JP4-039 administered to each animal was 400 Mice were immobilized for irradiation with intraperitoneal Nembutal anesthesia after administration of JP4-039 as described above. Mice were exposed to single-dose (29 Gy) or fractionated radiation (11.5 Gy per day for four days) to the upper body. Single-dose animals received F15 alone, MnSOD-PL 24 hours prior to irradiation, JP4-039 immediately before irradiation (15 mice per group). Fractionated animals received F15 alone, MnSOD-PL 24 hours prior to the first and third fractions, JP4-039 prior to each fraction (15 mice per group).

A separate group of mice received intratracheal administration of 1×10⁶ Lewis lung carcinoma cells (3LL) seven days prior to administration of JP4-039, followed immediately by excision of lung tissue to calculate JP4-039 uptake by cancer cells. Another group of animals also received administration of 3LL cells followed by either no treatment, F15 alone, JP4-039, or MnSOD-PL 24 hours prior to exposure to 20 Gy thoracic irradiation to determine whether JP4-039 were radioprotective to cancer cells as well.

To determine whether JP4-039 administration resulted in uptake of the compound by esophageal multipotent cells or differentiated epithelial cells, the stem cell-enriched side population (SP) was compared to non-SP (NSP) cells after isolation. Ten minutes after administration of JP4-039, esophagi were removed, minced, and incubated in a solution of 0.2% Collagenase type II, 0.3% Dispase and 0.025% Trypsin for 45 minutes at 37 degrees Celsius. Cell aggregates were then passed through sequentially smaller needles (to 23-gauge) and filtered with a 40 mM cell strainer into DMEM supplemented with 40% fetal bovine serum. Suspensions were pelleted via centrifugation and resuspended at 10⁶ cells/ml in pre-warmed DMEM (2% FBS, 10 mM HEPES). Cells were incubated in 6 μg/ml Hoechst 33342 for 90 minutes to identify SP cells. Verapamil, which inhibits the efflux of Hoechst, was used as a concentration of 50 mM for the purpose of cell gating. Cells were pelleted and resuspended in cold Hank's Balanced Salt Solution (HBSS) (2% FBS, 10 mM HEPES) and incubated with anti-CD45-phycoerythrin (PE)-fluorescein isothiocyanate (FITC) and/or anti-Ter119-PE-Cy7 antibodies at 1:200 dilutions, to discriminate hematopoietic cells. Antibody-treated cells were incubated on ice for 20 minutes, washed in cold HBSS, filtered, pelleted, and resuspended in cold HBSS. Propidium iodine was added at 2 μg/ml immediately prior to flow cytometry. SP and NSP cells were quantified, sorted into separate collection tubes containing cold HBSS (2% FBS, 10 mM HEPES) and pelleted. The supernatant was then aspirated and the cells snap-frozen in liquid nitrogen. JP4-039 content in sorted SP and NSP cells was quantified by electron paramagnetic resonance (EPR) analysis using a JEOL-RE1XEPR spectrometer.

Lastly, to determine whether JP4-039 was taken up by other tissue, esophagus, lung orthotopic tumor, liver, and peripheral blood samples were taken 10, 30, and 60 minutes after intraesophageal administration of the compound. Samples were snap-frozen on dry ice and JP4-039 content was quantified by EPR analysis.

Results.

JP4-039 Uptake in Normal Tissue and Orthotopic Tumors.

To determine if the orally-administered JP4-039 also reached other tissues and orthotopic tumors, esophagus, peripheral blood, bone marrow, liver, and 3LL orthotopic tumors were harvested at 10, 30, and 60 minutes after intraesophageal administration of JP4-039, followed by quantification by EPR. JP4-039 in liver peaked after 10 minutes at 122/2 pmol/mg protein and gradually decreased over time. JP4-039 levels in peripheral blood and orthotopic tumor peaked at 30 minutes at 51.1 and 276.0 pmol/mg protein, respectively. These data demonstrate that intraesophageal delivery of JP4-039 in F15 liposome formulation allows nitroxide uptake by both normal and tumor tissue. Lower levels of JP4-039 were detected in bone marrow up to 60 minutes after drug swallow compared to levels in liver and tumor tissue.

JP4-039 is Detected in Esophageal SP and NSP Cells.

The above data confirm and extend prior data showing detection of JP4-039 in esophagus by EPR. The next step is to determine whether the drug reaches esophageal stem cells. Twenty mouse esophagi were excised 10 minutes after intraesophageal delivery of JP4-039 with subsequent isolation of SP and NSP cells. Sorting results demonstrate that the 101.00 SP cell pellet contained 275.1 fmole JP4-039. The 3,387,00 sorted NSP cells contained 221.3 fmole JP4-039. The data establish that swallowed JP4-039 in F15 formulation reaches and is detectable in both excised and isolate SP and NSP cells.

JP4-039 is Radioprotective in Single Fraction Upper-Body Irradiated Mice.

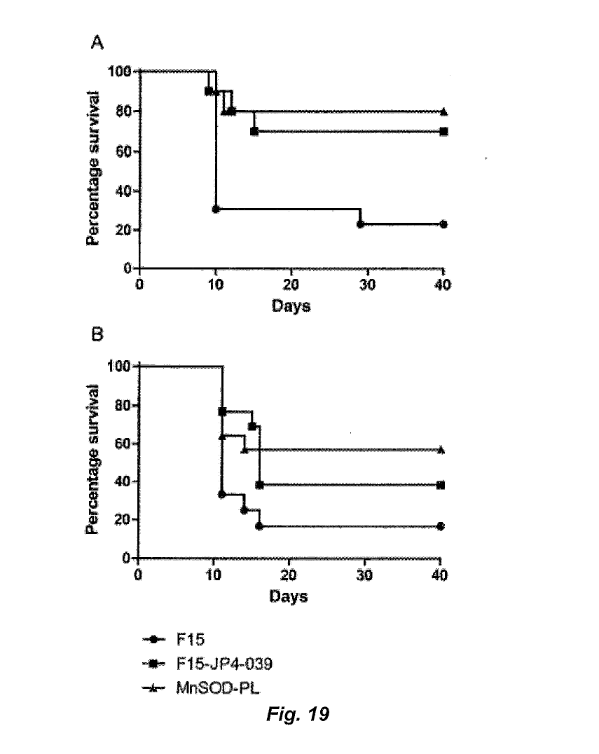

To determine whether intraesophageal administration of JP4-039 in F15 would ameliorate irradiation induced esophagitis in mice, mice were treated with F15 only or JP4-039 immediately prior to a single fraction of 29 Gy thoracic irradiation. As a positive control, MnSOD-PL was administered 24 hours prior to the irradiation. Mice that were treated with JP4-039 prior to 29 Gy thoracic irradiation demonstrated increased survival compared to the F15 vehicle only group (p=0.0384) [FIG. 19A]. The data indicate that intraesophageal administration of JP4-039 in F15 formulation ameliorates single-fraction irradiation-induced death from esophagitis.

JP4-039 is Radioprotective in Multiple-Fraction Upper Body-Irradiated Mice.

To evaluate radioprotection by JP4-039 in multiple-fraction upper-body irradiation, mice were treated with intraesophageal JP4-039 prior to each of four fractions of 11.5 Gy thoracic irradiation. MnSOD-PL was administered as a positive control 24 hours prior to the first and third fractions. Mice treated with JP4-039 prior to irradiation had increased survival compared to the F15 only control group (p=0.0388) [FIG. 19B]. The data indicate that JP4-039 is protective against fractionated irradiation of the esophagus and are effective when given in multiple administrations.

JP4-039 does not Protect Orthotopic Tumors from Radiation.

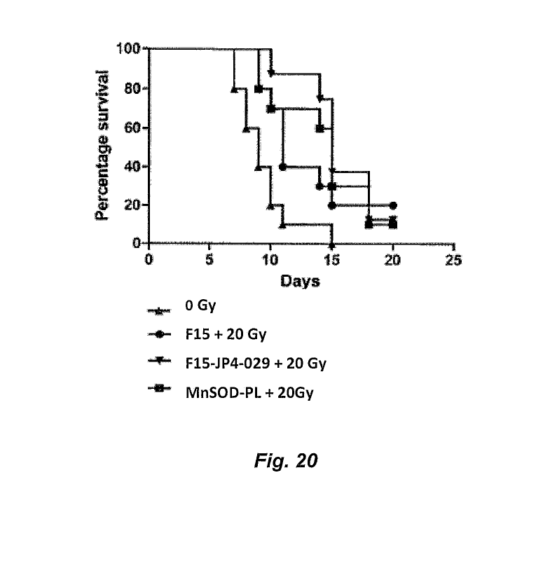

The above data indicate that JP4-039 was taken up by an orthotopic tumor after drug swallow. To determine whether the drug also protected tumors from irradiation damage, an orthotopic lung tumor model was used. Mice received intratracheal injection of 3LL cells 1 week prior to exposure to 20 Gy thoracic irradiation. This dose of irradiation was chosen to reduce tumor growth but was below the level required for lethal esophagitis. Irradiated mice were divided into treatment groups of F15, JP4-039 plus F15, and MnSOD-PL. Control tumor-bearing mice received no irradiation. Non-irradiated mice died rapidly of progressive tumor within 15 days; irradiated mice survived significantly longer due to reduction in tumor growth. Irradiated mice that received orally administered JP4-039, as well as those receiving positive control of MnSOD-PL prior to 20 Gy did not survive significantly differently compared to mice given F15 alone (p=0.3693) [FIG. 20]. The data show that JP4-039 does not protect tumors from irradiation.

Discussion.

The above results are significant in highlighting the advantage of the small molecule protector JP4-039 as an esophageal radioprotector over MnSOD-PL gene therapy, which has been the standard to this point. The small molecule protectors are relatively inexpensive to produce and do not require 24-hour administration to show efficacy. Instead, it can be given immediately prior to radiation therapy, and are quickly cleared from tissues. Further, administration of the drug in the F15 formulation, which shows low toxicity to cultured mammalian cells and good tolerability when administered to mice, is an effective method for preventing or mitigating the effects of irradiation-induced esophagitis.

### Example 9

### Assessment of Swallowed JP4-039 as an Effective Esophageal Radioprotector

JP4-039 in F15 Formulation is Given Prior to Each Fraction of Irradiation in One, Four, Six, or 28 Fractions are Tested in C57BL/6HNsd Mice.

Optimal dosing and time of administration are determined through analysis of levels of flurochrome labeled JP4-039 (BODIPY) in esophagus after swallow, dose is optimized when survival results equal that of MnSOD-PL administration. Experimental controls include TEMPOL in F15, F15 alone, MnSOD-PL, or radiation only. Active compound mice receive doses of JP4-039 ranging from 1 μg to 1 mg in tenfold increments in a constant volume of 110 μl of F15 formulation. Upper-body irradiation is by single fraction 28 Gy, four fraction 12 Gy daily for four days, six fraction 11 Gy daily for six days, 10 fraction 9 Gy for fourteen days, or clinically relevant 28 fraction 2.1 Gy for five and a half weeks. Active JP4-039 or control compounds are administered between each fraction, and six hours, twelve hours, and eighteen hours after each fraction, except for MnSOD-PL. Mitochondrial targeting by JP4-039 is confirmed by comparison of JP4-039 (BODIPY) with TEMPOL in whole esophagus tissue, in single cells, and at the mitochondrial level. Mitochondria-rich fractions are obtained by 10 min centrifugation at 5,000 g followed by 2× wash with isolation buffer. Pellets are then washed twice with incubation buffer and analyzed using microscopy for levels of BODIPY in single cells, per mg of tissue, and for nitroxide by EPR. Esophageal fibrosis is also analyzed essentially in the manner described in Epperly M W, et al. Mitochondrial targeting of a catalase transgene product by plasmid liposomes increases radioresistance in vitro and in vivo. Radiation Res. 2009, 171: 588-595.

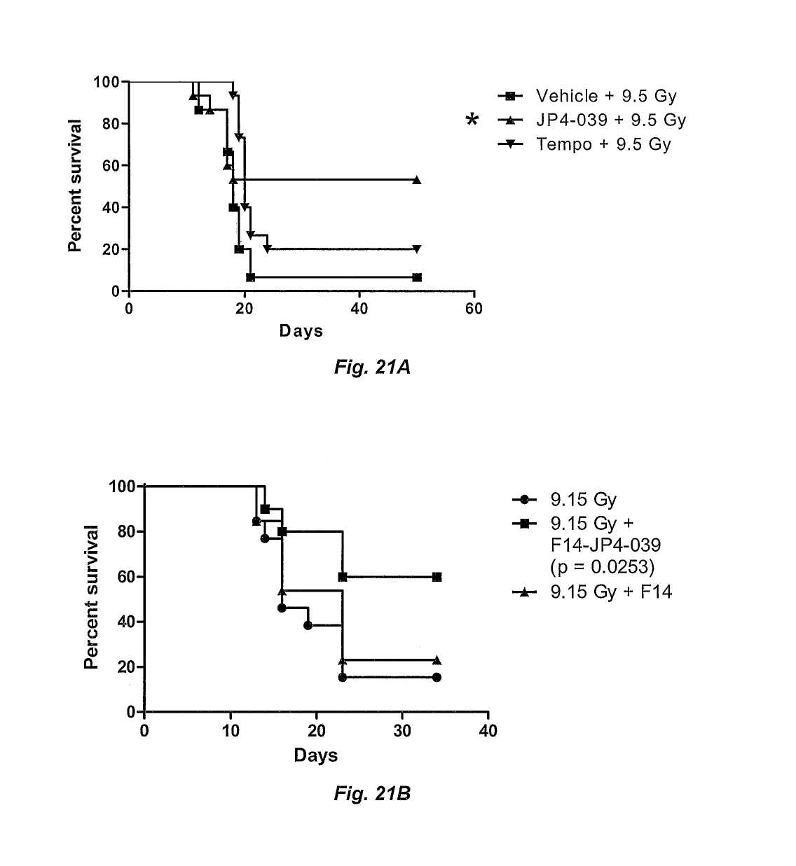

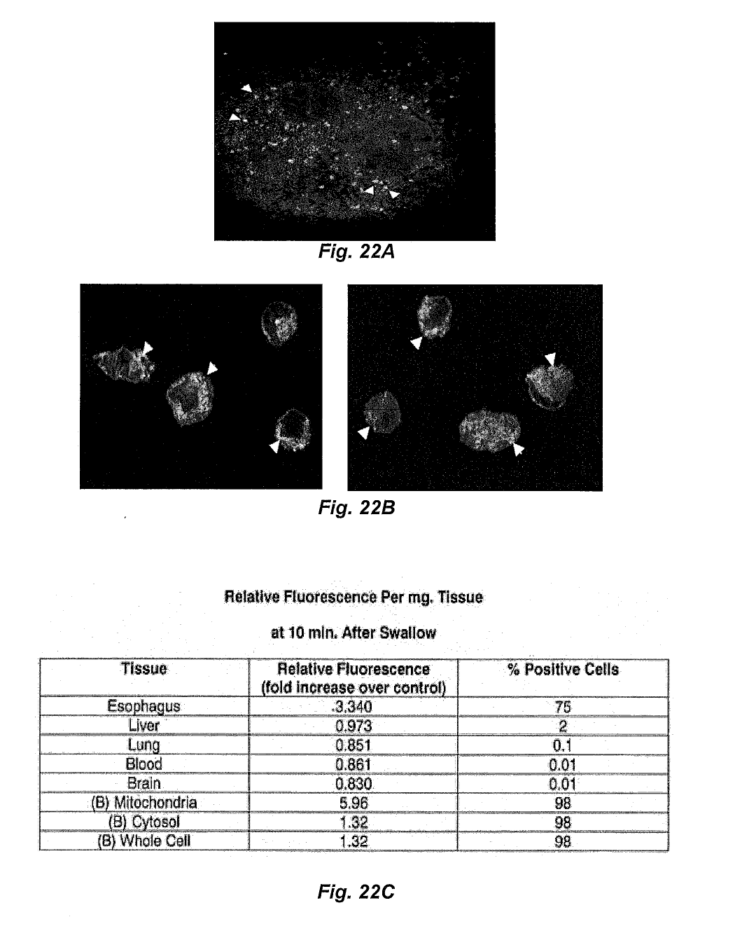

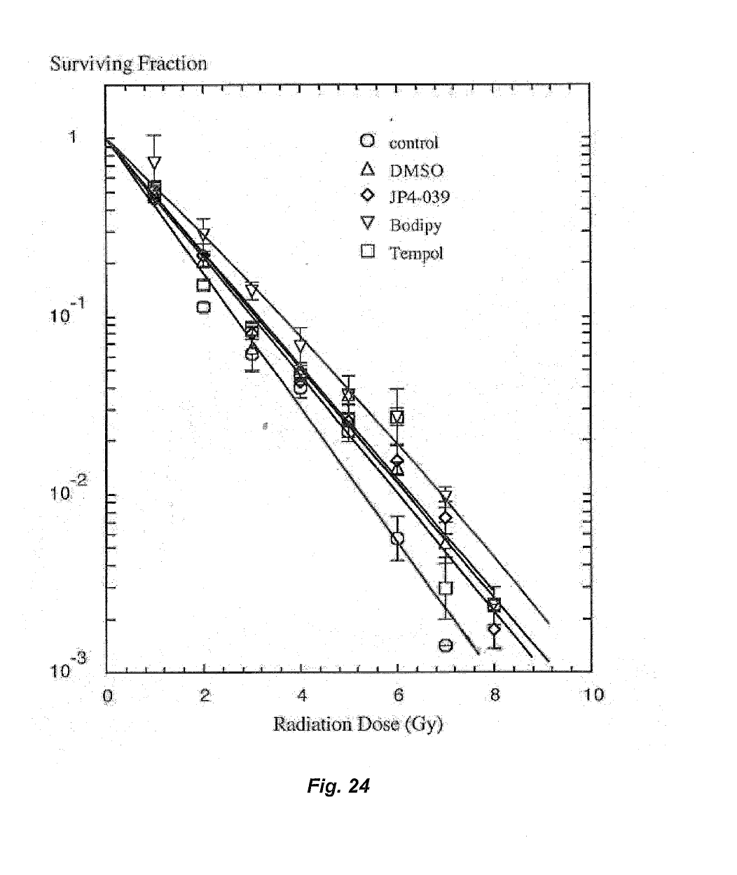

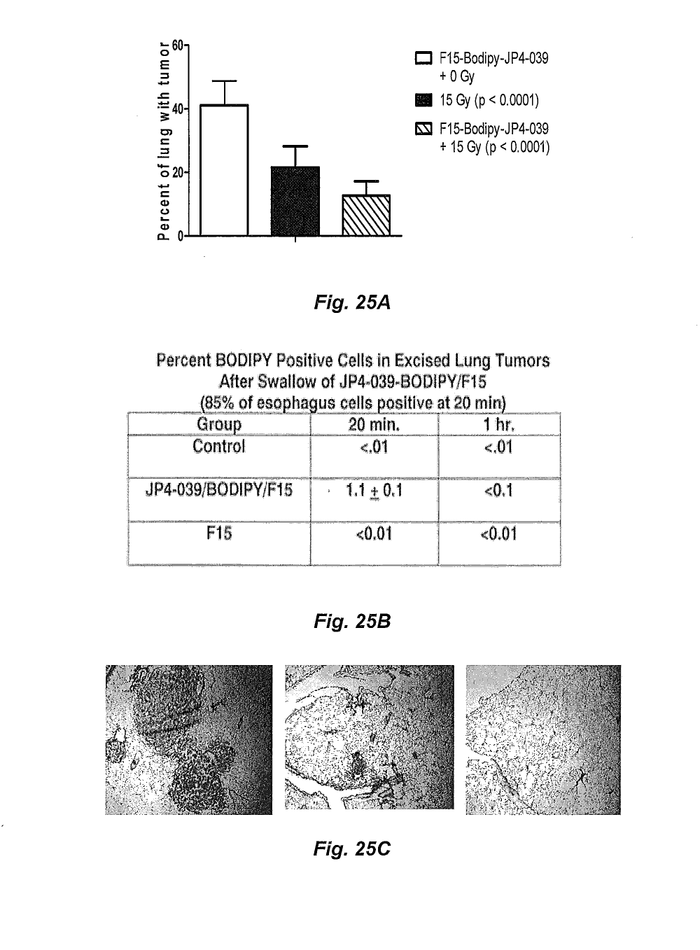

The above methods are based on preliminary data showing that JP4-039 administered via i.p. injection at 10 mg/kg 10 minutes before either 9/5 Gy [FIG. 21A] or 9.15 Gy [FIG. 21B] total-body irradiation protected C57BL/6NHsd female mice from esophagitis-induced death compared to those that received either TEMPO or F14 alone (p=0.0301 and p=0.010, respectively). Additionally, incubation of 32D cl3 cells in 10 μM JP4-039 for one hour increased survival following exposure to 0-8 Gy irradiation [FIG. 24]. Lastly, targeting of mitochondria was confirmed by use of a fluorochrome labeled JP4-039 (BODIPY) administered in F15 formulation by swallowing to C57BL/HNsd mice in a concentration of 4 mg/kg, followed 10 minutes later by excision of esophagi, liver, lung, and brain tissue, as well as a blood sample, for imaging. Mitochondrial labeling was accomplished in vitro with use of Mitotracker and JP4-039 (BODIPY) in esophageal cell line. imaging of esophageal cell lines in vitro, revealing co-localization of signals in mitochondria [FIG. 22A-C]

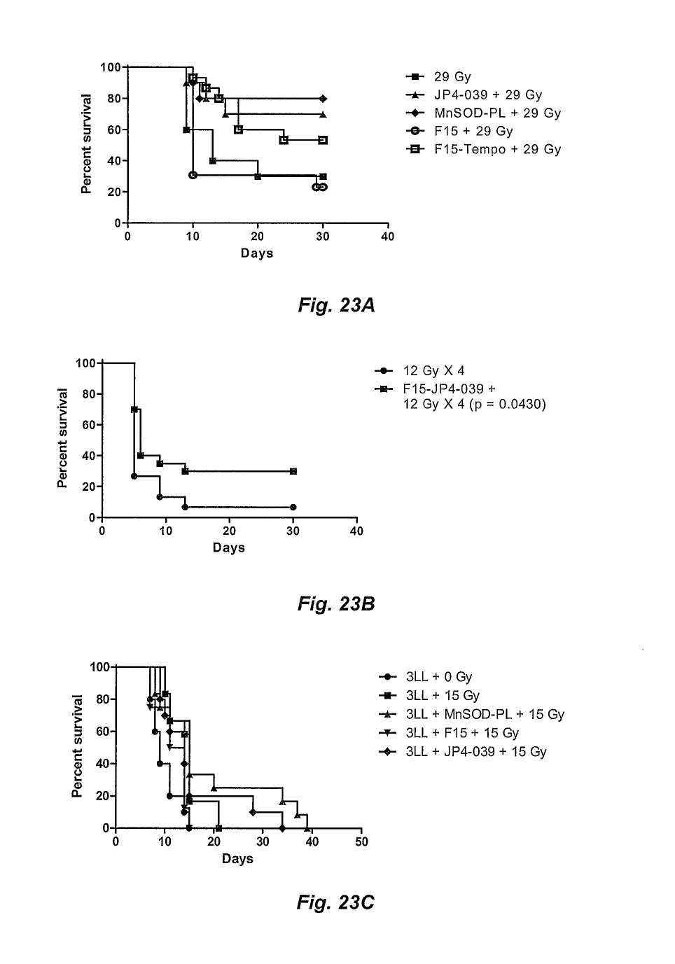

Further support for the above methods is found in additional preliminary data showing that 4 μg of JP4-039 in 110 μl of F15 administered 10 minutes before and MnSOD-PL administered 24 hours prior to either 29 Gy upper body irradiation [FIG. 23A] or four daily sessions of 12 Gy [FIG. 23B], was protective in C57BL/6NHsd mice, as compared to F15 or radiation alone (p=0.0430 in fractionated irradiation). To demonstrate that this protection did not translate to underlying tumor cells that are the target of irradiation, a further experiment tested whether JP4-039 would protect orthotopic Lewis Lung Carcinoma (3LL) cells from irradiation. Ten minutes after intraesophageal swallow of JP4-039 in F15 formulation (absolute amount of 4 μg in 110 μl of F15), or F15 alone, or 24 hours prior to administration of MnSOD-PL, C57BL/6HNsd mice were exposed to 0 or 15 Gy of upper-body irradiation. Results demonstrated that JP4-039 did not protect 3LL cells from irradiation [FIG. 23C].

### Example 10

### Analysis of JP4-039 Protection of Intrinsic and Marrow-Derived Progenitor Cells in Irradiated Esophagus

Initially, JP4-039 (BODIPY) is administered at the optimized dose from Experiment 10 prior to 28 Gy upper-body irradiation. Esophagus SP cells are removed at 10, 30, and 60 minutes after irradiation and assayed for flurochrome labeling. The experiment is then repeated with the same optimized dose and timing from Experiment 10 (above), followed by irradiation for 4, 6, 10, and 28 fractions. Five days after the first fraction of irradiation, female C57BL/6JHNsd mice receive marrow transplants from GFP+ male mice. After all irradiation fractions are completed, esophagi are removed, SP cells separated, and the GFP+ subset is sorted. BODIPY signaling is then scored in mitochondria of GFP+ cells. Additionally, female mice, stably chimeric for male GFP+ bone marrow, are utilized in the same procedure described previously. Control animals receive F15 alone, TEMPOL in F15, MnSOD-PL, and irradiation alone. In another experiment, imaging of JP4-039 in marrow mitochondria is accomplished using a novel tagged compound, JP4-039 (BODIPY-R6G).

Further experiments involve determining the level of swallowed JP4-039 that reaches marrow, lung, liver, and brain, as detailed in Example 10. Lastly, the protective effects of JP4-039 (BODIPY-R6G) in chimeric female mice are assessed. Esophageal cells are removed at serial time points beginning at day 5 after irradiation in the 4, 6, 10, and 28 fraction studies, and again after JP4-039 (BODIPY-R6G) in F15 swallow (10 minutes to 3 hours after swallow). GFP+ subpopulation is separated, SP vs. non-SP cells are separated and imaged for BODIPY and Mitotracker for fluorescence per mg of tissue.

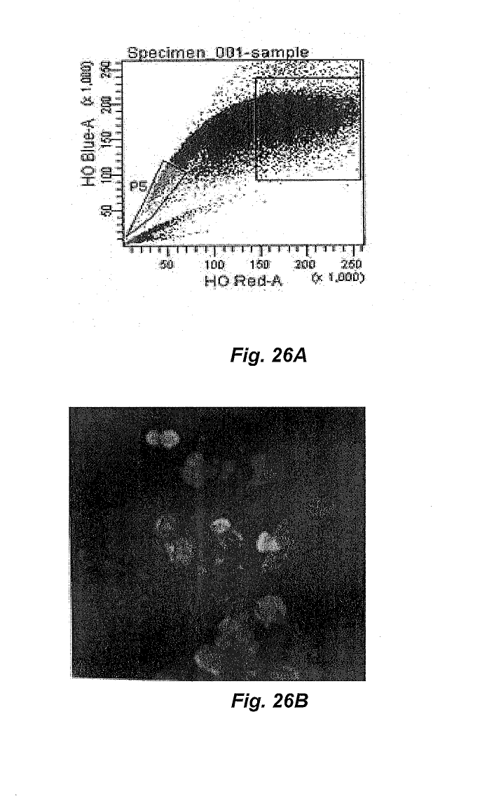

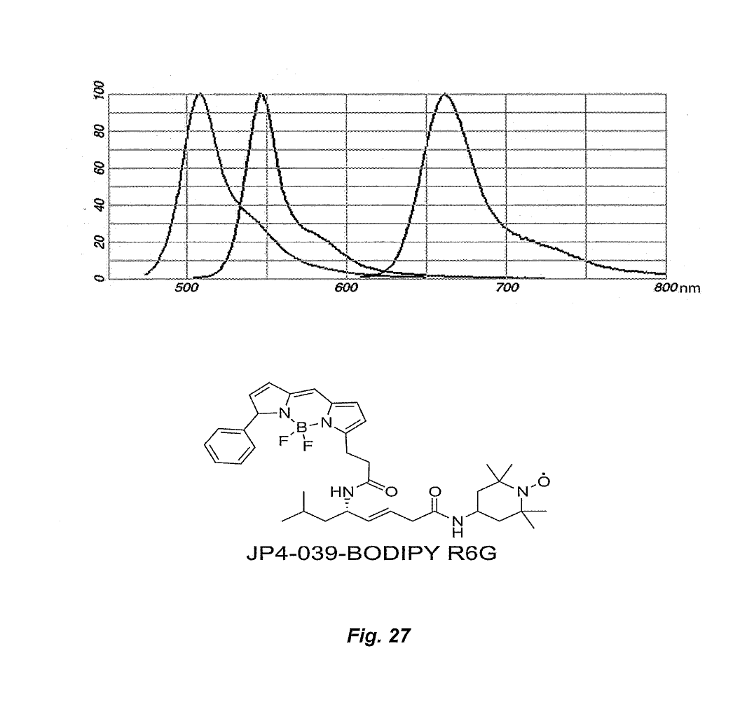

The above methods are based on preliminary data showing that swallowed JP4-039 reaches SP cells. Additional data show that swallowed JP4-039 nitroxide reaches GFP+ esophageal (SP) stein cells in GFP+ marrow chimeric mice. FIG. 26A shows JP4-039/BODIPY-R6G/F15 in esophageal SP population of GFP+ marrow chimeric mice 5 days after 29 Gy, then drug swallow, and immediate esophagus removal. (The cell sorting diagram of control non-irradiated, non-chimeric esophagus showed 56,000 SP cells out of 1 million sorted (0% GFP+). In FIG. 26A, there were 60,000 SP cells, 10% GFP+, (P5) out of 1 million in GFP+ marrow chimeric mice esophagus. In FIG. 26B, immunohistochemical analysis of multilineage colony from single GFP+JP4-039/BODIPY/F15 treated esophageal SP cell—p5—is shown. Cells were grown in 0.8% methylcellulose-containing media. At day 14, the methylcellulose-containing media was removed and the remaining adherent cells were fixed in methanol and stained with antibodies to Sca-1, CD45, F4/80, endothelin, and Vimentin. Colony demonstrates cells positive for endothelin (green in original), F4/80-positive macrophages (red in original), and vimentin (yellow in original). By use of JP4-039 BODIPY-R6G [FIG. 27], these data demonstrate the feasibility of this approach to determine that JP4-039 is protecting intrinsic, as well as marrow origin, GFP+ esophageal stem cells in vivo.

### Example 11

### Determination of Whether Swallowed JP4-039 Protects Transgenic Lung Tumors

The methods described herein relate to determining whether JP4-039 is also protective to transgenic lung tumors. C57BL/6J-K-ras transgenic mice (as well as LSL-K-ras mice) to keep mouse strain consistent with published data, are used. Female mice chimeric for male GFP+ bone marrow are administered CRE-recombinase to induce lung tumors, then treated in 5 protocols: 1) single fraction, 2) four, 3) six, 4) ten fraction and 5) clinical 2.1 Gy×28 fractionated thoracic irradiation. Each fraction is preceded by swallow of JP4-039 (BODIPY) in F15 formulation compared to F15 formulation alone. The statistical consideration is whether improved healing of esophageal radiation damage by JP4-039 correlates with increased numbers of intrinsic and/or bone marrow derived GFP+SP cells in the sections of transgenic tumors. Measure of JP4-039 (BODIPY) uptake in the esophagus is correlated to the effects on tumors by each of several parameters: 1) decrease in acute irradiation-induced esophageal apoptosis, 2) inflammatory cytokines level, and 3) late stricture. Secondly, esophageal radiation protection by JP4-039 is investigated for protection of transgenic tumors. Finally, optimized swallowed JP4-039 in F15 is combined with radiation dose escalation to radio-control tumors and then hold mice to measure late esophageal stricture or unexpected esophageal tumors in the manner described in (Epperly M W, et al. Mitochondrial targeting of a catalase transgene product by plasmid liposomes increases radioresistance in vitro and in vivo. Radiation Res. 2009, 171: 588-595).

The above methods are carried out as follows. JP4-039/BODIPY/F15 Esophageal Radioprotection Effect on LSL-K-ras and C57BL/6-K-ras Mouse Tumors:

Single Fraction:

Optimal esophageal protective dose and time of administration of JP4-039/F15 is used in mice harboring tumors allowing quantitation of mouse survival and surviving explanted tumor colony forming cells. Mice (n=15) receive thoracic irradiation 10 minutes after swallow of JP4-039/F15, tumors are removed, at 24 hrs., 48 hrs., or 7 days afterwards, single cell suspension is derived, fluro-JP4-039 (BODIPY) levels measured at 1 day, and 7 day colonies counted.

### Four, Six, Ten, and Twenty-Eight Fraction Irradiation:

In sub-groups of mice (n=15), esophagus and tumor are removed after each 12 Gy or 11 Gy radiation fraction, and fluro-JP4-039-(BODIPY) uptake in esophagus and tumor compared by fluorochrome labeling. Controls include mice receiving MnSOD-PL, Tempol/F15, and F15 alone.

### Late Effects, Chemoradiotherapy, and Radiation Dose Escalation:

The effect of JP4-039/F15 on radiation esophageal inflammation is determined with histopathology and biomarkers including TGFβ, IL-1, TNFα as indicators of radiotherapy esophagitis and late fibrosis at 100-120 days. Esophagus is removed after the last irradiation fraction and single cell suspensions analyzed by RT-PCR robot for inflammatory cytokine markers. The effect of JP4-039/F15 in mice receiving Carboplatinum/Taxol and fractionated irradiation is tested according to published methods (Epperly M W, et al. Mitochondrial targeting of a catalase transgene product by plasmid liposomes increases radioresistance in vitro and in vivo. Radiation Res. 2009, 171: 588-595), mice are held for six months and esophageal stricture quantitated according to published methods using excised esophagus and Mallory-Trichrome staining for fibrosis (Epperly M W, et al. Mitochondrial targeting of a catalase transgene product by plasmid liposomes increases radioresistance in vitro and in vivo. Radiation Res. 2009, 171: 588-595). Finally, radiation dose escalation is carried out (10% dose per fraction increases until dose limiting toxicity) using optimized time and dose administration of JP4-039 in the setting of transgenic LSL-K-ras tumors.
Measurement of Antioxidant Stores Inflammatory Cytokine mRNA Levels in JP4-039/F15 Treated Mice:
Esophageal tissue removed at serial times after single fraction irradiation is tested for antioxidants, and by robot RTPCR, for acute inflammatory cytokine markers of irradiation damage including TNFα, IL-1, TGFβ as described above for JP4-039/F15 treated mice. The relative radioprotective effect in tumors is compared to the effect of JP4-039/F15 in esophagus and compared to F15 alone, or irradiation alone.

### Determine Whether JP4-039 (BODIPY) Increases Intrinsic and Marrow Origin Esophageal SP Cells in Tumor Bearing Mice.

Groups of female LSL-K-ras or C57BL/6-K-ras mice (n=15), control, or GFP+ marrow chimeric mice receive JP4-039/BODIPY-R6G/F15, F15 alone, or irradiation alone in single fraction, 4, 6, 10, or 28 fraction experiments. Other mice receive at day 5 after 1^{st} fraction injection of 1×10⁷ sex-mismatched GFP+ male bone marrow. At serial time points after the last irradiation fraction and last JP4-039 administration, esophageal specimens are removed, single cell suspensions prepared, whole esophagus, non-SP, and SP populations evaluated for JP4-039 (BODIPY-R6G) in the GFP+ fraction.

### Quantitation of JP4-039 Effect on Therapeutic Tumor Irradiation, and Survival:

LSL-K-ras or C57BL/6-K-ras mice with carcinomas are treated prior to single fraction 28 Gy upper body irradiation with swallowed JP4-039/BODIPY-R6G/F15, F15 emulsion alone, or irradiation alone and each fractionation scheme of 4, 6, 10, 28 fractions. Tumors are removed at serial times (n=5 per group), and single cells suspensions tested for JP4-039-BODIPY-R6G positive tumor cells and in chimeric mice for GFP+ cells.

### Radiation Dose Escalation:

Optimal JP4-039 in F15 swallow(s) are followed by increasing single fraction and fractionated irradiation doses in 10% increments. The dose modifying effect is calculated. Chemoradiotherapy of LSL-K-ras or C57BL/6-K-ras tumors with higher radiation doses follow. Effects on esophageal stricture at day 60 are quantitated as in Experiment 10.

### Quantitation of Bone Marrow Derived Cells in Irradiated Tumors:

Preliminary data demonstrate low or undetectable GFP+ cells in transgenic tumors of JP4-039 (BODIPY-R6G) swallow treated mice [FIG. 27B]. Experiments with C57BL/6-K-ras female mice receiving male GFP+ bone marrow or chimeric for C57BL/6-GFP+ male marrow (n=15) are carried out removing tumors at four time points after completion of the last irradiation fraction optimized in Experiment 10 (single, four, six, ten, or twenty-eight fraction), and the number of GFP+ cells in tumor relative to the surviving fraction of clonogenic tumor cells determined Total cells in the explant are counted by Coulter Counter. GFP+ hematopoietic origin cells in tumors is sorted and analyzed by hematologic and histochemical staining according to methods known to those of ordinary skill in the art. The data establish that JP4-039 (BODIPY-R6G) in F15 emulsion is an esophageal radioprotective agent that increases intrinsic esophageal stein cell protection, enhances migration into the esophagus of bone marrow progenitors of esophageal squamous epithelium, and does not protect C57B1/6-K-ras transgenic mouse tumors.

The above methods are based on preliminary data showing that intraesophageal swallow of JP4-039 (BODIPY) in F15 formulation protects esophagus but not transgenic LSL-K-ras induced lunch cancer from irradiation. Intraesophageal JP4-039 (BODIPY) in F15 formulation+15 Gy thoracic irradiation or 15 Gy thoracic irradiation alone significantly decreased percent of lung tumor (p<0.0001 and p<0.0001, respectively) [FIG. 27A, C]. Swallowed JP4-039 (BODIPY) did not decrease the therapeutic effect of irradiation. Additionally, the animals receiving JP4-039 (BODIPY) showed only low levels of BODIPY+ cells in tumors [FIG. 27B], suggesting that JP4-039 (BODIPY) in F15 formulation does not reach LSL-K-ras lung tumors when administered intraesophageally, while still protecting from esophagitis. Irradiated mice showed similar results to those who did not receive irradiation.

Whereas particular embodiments of this invention have been described above for purposes of illustration, it will be evident to those skilled in the art that numerous variations of the details of the present invention may be made without departing from the invention as defined in the appended claims.

1. A method of preventing or mitigating ionizing irradiation-induced esophagitis in a subject, comprising administering to the esophagus of a subject prior to, during or after exposure of the subject to radiation, a composition comprising an amount of a compound effective to prevent, mitigate or treat radiation injury in the subject; wherein the compound is chosen from one of: 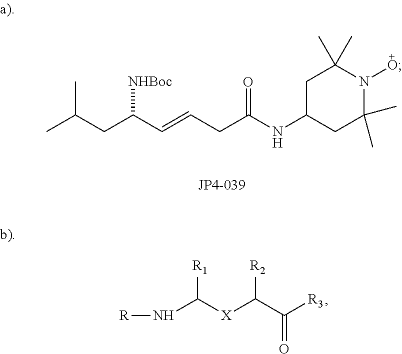
wherein X is one of 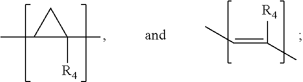
R₁ and R₂ are hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-,chloro- or fluoro-substituted; R₃ is —NH—R₅, —O—R₅ or —CH₂—R₅, and R₅ is an —N—O., —N—OH or N═O containing group; R is —C(O)—R₆, —C(O)O—R₆, or —P(O)—(R₆)₂ wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl-, chloro- or fluoro-substituted;
c). a compound having the structure (i) R1-R2-R3 or (ii) R1, in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; and 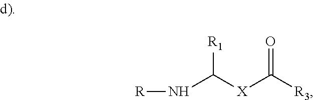
wherein X is one of 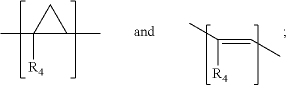
R₁ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁-C₆ straight or branched-chain alkyl, or a C₁-C₆ straight or branched-chain alkyl further comprising a phenyl(C₆H₅) group, that is unsubstituted or is methyl-,hydroxyl-, chloro- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C1-C6 straight or branched-chain alkyl further comprising one or more phenyl (—C₆H₅) groups that are independently unsubstituted, or methyl-, ethyl-, hydroxyl-, chloro- or fluoro-substituted.

2. The method of claim 1, the compound having the structure 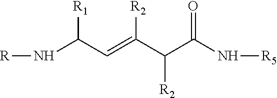
or the structure

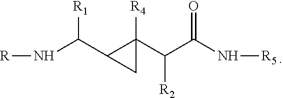

3. The method of claim 2, the compound having the structure

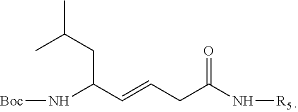

4. The method of claim 3, the compound having the structure

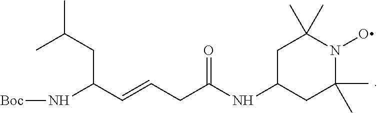

5. The method of claim 1, the compound having the structure 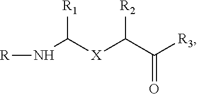
in which R is Ac, Boc, Cbz, or —P(O)-Ph₂.

6. The method of claim 1, the compound having the structure

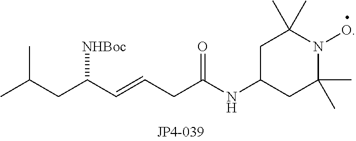

7. The method of claim 1, the compound having the structure 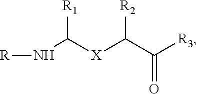
in which R₁, R₂, R₄, and R₆ are independently chosen from hydrogen, methyl, ethyl, propyl, 2-propyl, butyl, t-butyl, pentyl, hexyl, benzyl, hydroxybenzyl, phenyl and hydroxyphenyl.

8. The method of claim 1, the compound having the structure 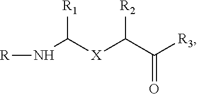
wherein when X is —CH═CR₄—, R₄ is hydrogen, methyl or ethyl.

9. The method of claim 1, the compound having the structure 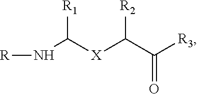
in which R₅ is 2,2,6,6-Tetramethyl-4-piperidine 1-oxyl, 1-methyl azaadamantane N-oxyl, or 1,1,3,3-tetramethylisoindolin-2-yloxyl.

10. The method of claim 1, the compound having the structure 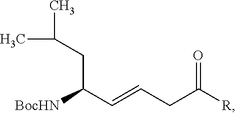
or the structure 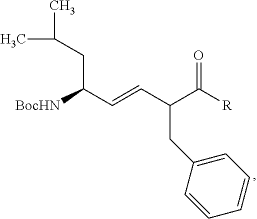
in which R is —NH—R₁, —O—R₁ or —CH₂—R₁, and R₁ is an —N—O., —N—OH or N═O containing group.

11. The method of claim 1, the compound having the structure 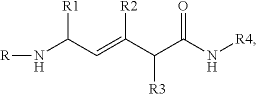
in which R1, R2 and R3 are, independently, hydrogen, C₁-C₆ straight or branched-chain alkyl, or C₁-C₆ straight or branched-chain alkyl including a phenyl(C₆H₅) group that is unsubstituted, methyl-, hydroxyl-, chloro- or fluoro-substituted; R4 is an —N—O., —N—OH or N═O containing group; and R is —C(O)—R5, —C(O)O—R5, or —P(O)—(R5)₂, wherein R5 is C₁-C₆ straight or branched-chain alkyl, or C₁-C₆ straight or branched-chain alkyl including a phenyl(Ph,C₆H₅) group that is unsubstituted, methyl-, hydroxyl-, chloro- or fluoro-substituted.

12. The method of claim 11, in which R is Ac, Boc, Cbz, or —P(O)-Ph₂.

13. The method of claim 11 in which R1, R2 and R3 independently are methyl, ethyl, propyl, 2-propyl, butyl, t-butyl, pentyl, hexyl, benzyl, hydroxybenzyl, phenyl and hydroxyphenyl.

14. The method of claim 11, in which R4 is 2,2,6,6-Tetramethyl-4-piperidine 1-oxyl, 1-methylazaadamantane N-oxyl), or 1,1,3,3-tetramethylisoindolin-2-yloxyl.

15-20. (canceled)

21. The method of claim 1, the compound having the structure:

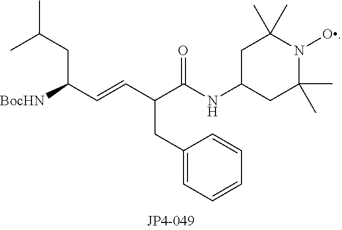

22. The method of claim 1, the compound having the structure:

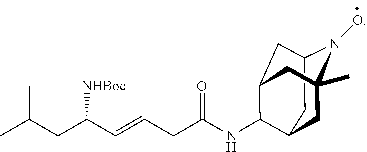

23. The method of claim 1, the compound having the structure:

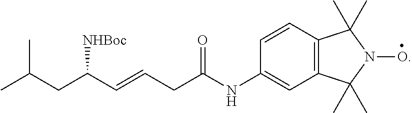

24-26. (canceled)

27. The method of claim 1, in which the compound is selected from the group consisting of: XJB-5-131, XJB-5-125, XJB-5-197, XJB-7-53, XJB-7-55, XJB-7-75, JP4-049, XJB-5-208, JED-E71-37, JED-E71-58.

28. The method of claim 1, in which the amount effective to prevent or mitigate ionizing irradiation-induced esophagitis in the subject ranges from 0.1 to 100 mg/kg in the subject.

29-31. (canceled)

32. The method of claim 1, in which the compound is administered between 30 minutes and one hour after radiation exposure in the subject.

33. The method of claim 1, in which the compound is administered prior to radiation exposure in the subject.

34. The method of claim 1 in which the compound is formulated in an oral liquid dosage form.

35. The method of claim 34, in which the oral liquid dosage form is a multi-phase liquid.

36. The method of claim 35, in which the multi-phase liquid is a liposome preparation.

37. The method of claim 35 in which the multi-phase liquid preparation comprises the compound, a phospholipid, a non-ionic surfactant, a cationic lipid and an aqueous solvent.

38. The method of claim 37, in which the cationic lipid is selected from the group consisting of DC-Cholesterol, DOTAP, DODAP, DDAB, ethyl-PC, DOTMA, and mixtures thereof.

39. The method of claim 35 in which multi-phase liquid comprises the compound, a phosphatidyl choline, a non-ionic detergent, a cationic lipid and an aqueous solvent.

41-49. (canceled)

50. A multi-phase or liposome composition comprising:
(a) a compound chosen from one of: 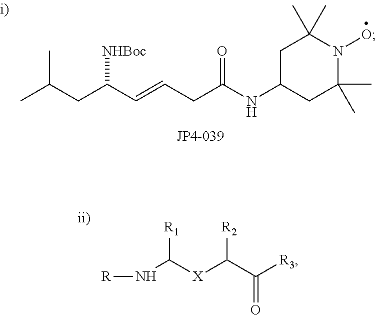
wherein X is one of 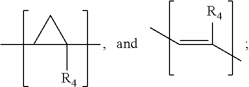
R₁ and R₂ are hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₃ is —NH—R₅, —O—R₅ or —CH₂R₅, and R₅ is an —N—O., —N—OH or N═O containing group; R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl-, chloro- or fluoro-substituted;
iii) a compound having the structure (i) RI—R2-R3 or (ii) R1 in which R1 and R3 are the same or different and have the structure —R4-R5, in which R4 is a mitochondria targeting group and R5 is —NH—R6, —O—R6 or —CH₂—R6, wherein R6 is an —N—O., —N—OH or N═O containing group and R4 and R5 for each of R1 and R3 may be the same or different; and R2 is a linker; and 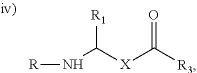
wherein X is one of 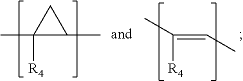
R₁ is hydrogen, C₁-C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₄ is hydrogen, C₁C₆ straight or branched-chain alkyl, or a C₁-C₆ straight or branched-chain alkyl further comprising a phenyl(C₆H₅) group, that is unsubstituted or is methyl-, hydroxyl-, chloro- or fluoro-substituted; R₃ is —NH—R₅, —O—R₅ or —CH₂—R₅, and R₅ is an —N—O., —N—OH or N═O containing group; and R is —C(O)—R₆, —C(O)O—R₆, or —P(O)—(R₆)₂, wherein R₆ is C₁-C₆ straight or branched-chain alkyl or C₁-C₆ straight or branched-chain alkyl further comprising one or more phenyl (—C₆H₅) groups that are independently unsubstituted, or methyl-, ethyl-, hydroxyl-, chloro- or fluoro-substituted;
(b) a phospholipid;
(c) a non-ionic detergent;
(d) a cationic lipid; and
(e) an aqueous solvent.

51. The composition of claim 50, consisting essentially of the compound, a phospholipid, a non-ionic detergent a cationic lipid and the aqueous solvent.

52. The composition of claim 51, in which the cationic lipid is a glutamic acid dialkyl amide.

53. The composition of claim 52, in which in which the glutamic acid dialkyl amide is L-glutamic acid-1,5-dioleyl amide.

54. The composition of claim 51, in which the phospholipid is a phosphatidyl choline.

55-56. (canceled)

57. The composition of claim 50, consisting essentially of the compound, soy phosphatidyl choline, L-glutamic acid-1,5-dioleyl amide, Tween 80 and the aqueous solvent.

58. A multiphase or liposome composition consisting essentially of soy phosphatidyl choline, Tween 80, L-glutamic acid-1,5-dioleyl amide (approximately 4:1:1 w/w), and an aqueous solvent with 8 mg/ml JP4-039.

59-61. (canceled)
